(19) United States
(12) Patent Application Publication
(10) Pub. No.: US 2003/0099932 A1
(43) Pub. Date: May 29, 2003

(54) Retroviral vectors with separation sequences

(76) Inventors: James B. Lorens, Portola Valley, CA (US); David A. Ferrick, El Macero, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/139,146

(22) Filed: May 3, 2002

Related U.S. Application Data
(63) Continuation-in-part of application No. 09/966,976, filed on Sep. 27, 2001.
Application No. 09/966,976 is a continuation of application No. 09/963,206, filed on Sep. 25, 2001.
Application No. 09/966,976 is a continuation of application No. 09/963,247, filed on Sep. 25, 2001.
Application No. 09/963,247 is a division of application No. 09/076,624, filed on May 12, 1998.

(51) Int. Cl. C12Q 1/70; C12Q 1/68; C12N 7/00; C12N 15/867

(52) U.S. Cl. 435/5; 435/6; 435/456; 435/235.1; 435/320.1

(57) ABSTRACT

The invention relates to retroviral vectors comprising fusion nucleic acids useful for expressing a plurality of separate proteins products encoded by genes of interest. The invention further relates to use of the compositions in methods for screening for candidate agents producing an altered phenotype in cells.

### CROSS-REFERENCE TO RELATED APPLICATIONS

This is a continuation-in-part application of U.S. application Ser. No. 09/076,624, filed May 12, 1998 and application entitled “Methods and Compositions Comprising Renilla GFP” filed Apr. 24, 2002 (U.S. Ser. No. not available). The content of each of these applications is hereby incorporated by reference in their entirety.

### FIELD OF THE INVENTION

The present invention relates to compositions and methods for use of separation sequences to express a plurality of gene products in cells. The invention further relates to use of the compositions in screens for effectors of cell physiology, screens for agents directed against pathogens, and in gene therapy applications.

### BACKGROUND OF THE INVENTION

Expressing multiple gene products within a single cell or organism has a variety of important applications in biology and medical therapeutics. It is useful in monitoring expression of genes of interest, expression of gene products unaffected by physical linkage to other proteins, expression of functional proteins acting as heteromultimers, and in gene therapy where the therapeutic agent comprises a multigene product. Expressing multiple gene products is also useful in developing screening methods for identifying agents that affect various cellular regulatory pathways or in screens for agents directed against pathogenic processes.

One traditional method for expressing different gene products in cells or organisms entails use of fusion gene constructs to produce a chimeric protein. Fusion proteins are useful for monitoring the expression of the fused protein, expressing a single peptide having two different protein activities, and localizing proteins to distinct subcellular compartments. However, there are several limitations associated with fused proteins. These include, among others, loss of activity of the individual proteins and different cellular localizations of the fused peptides, which may be incompatible with their cellular function.

Another method for expressing multiple gene products involves use of separate, independent promoters to express each gene of interest. This approach may require use of at least two different vectors, each expressing one gene of interest under control of their own promoters, or use of a single vector having multiple promoters where each promoter drives the expression of a gene of interest. Although these strategies obviate some of the difficulties and limitations of fusion proteins, there are attendant problems relating to promoter suppression, promoter interference, and gene rearrangements that result in inconsistent expression. In addition, when each gene of interest are on separate vectors, some cells will express one gene product but not the other since introducing the vectors into the cells rely on probabilistic distribution of vectors.

An alternative method relies on manipulating RNA splicing signals to produce different mRNA splice products which encode different genes of interest. Although the method allows expression of multiple gene products from a single promoter, use of splicing signals may result in unequal level of the differently spliced RNA species and inefficient expression of the gene products of interest. Additional complications include the difficulty of engineering multiple splicing signals to express a plurality of gene products and the complications arising from cryptic splicing signals that become activated when placed in different sequence contexts.

Organisms have evolved a number of different strategies to express multiple proteins from a single transcript. These mechanisms rely on separation sequences acting at the RNA level on the cellular translation machinery or at the protein level by direct action on the translated protein to produce a plurality of proteins from a single transcript. Accordingly, the present invention provides retroviral vectors comprising separation sequences for co-expressing a plurality of genes of interest to express separate protein products under the control of a single promoter. These compositions find use in screens for candidate agents that modulate various aspects of cell physiology. In addition, since the separation sequences themselves are involved in cell regulation and pathogenic processes, the present invention also provides for methods of screening for agents that affect these separation reactions.

### SUMMARY OF THE INVENTION

In accordance with the objects outlined above, the present invention provides compositions and methods for expressing a plurality of separate gene products and methods of screening for candidate bioactive agents that alter the phenotype of a cell.

In one aspect, the composition comprises retroviral vectors comprising fusion nucleic acids comprising a promoter, different first gene of interest, separation sequence, and second gene of interest. The separation sequence provides a basis for producing separate protein products encoded by the genes of interest, which may comprise reporter genes or selection genes.

In another aspect the gene of interest comprises a nucleic acid encoding a dominant effector protein. Expression of the dominant effector protein alters the phenotype of the cell, which are then useful in screening for candidate bioactive agents that alter the phenotype produced by the dominant effector protein.

As the invention provides for methods of screening for bioactive candidate agents, the present invention also provides for fusion nucleic acids comprising a promoter, a different first gene of interest, separation sequence, and a second gene of interest, wherein the gene or genes of interest comprises candidate agents comprising cDNAs, genomic DNA fragments, or nucleic acids encoding randomized peptides. In some instances, the candidate nucleic acids do not encode peptides.

For constructing fusion nucleic acids of the present invention, the invention further provides for retroviral cloning vectors. The cloning vectors comprise a fusion nucleic acid comprising a promoter, multiple cloning site, separation sequence, and a second gene of interest, wherein second gene of interest may comprise a reporter or selection gene, or a second multiple cloning site.

In an additional aspect, the present invention provides for nucleic acid libraries and cellular libraries of retroviral vectors comprising the fusion nucleic acids of the present invention.

In another aspect, the present invention provides methods for screening candidate bioactive agents capable of altering the phenotype of a cell. The method comprises the steps of adding candidate agents to a plurality of cells expressing fusion nucleic acids comprising a different first gene of interest, separation sequence, and second gene of interest, and screening the plurality of cells for a cell exhibiting an altered phenotype, wherein the altered phenotype is due to the presence of the candidate bioactive agent. The methods may also include the steps of isolating the cell(s) exhibiting the altered phenotype and identifying the candidate bioactive agent producing the altered phenotype.

The present invention provides these retroviral vectors, retroviral libraries, cellular libraries, and compositions for screening candidate bioactive agents in the form of kits.

### BRIEF DESCRIPTION OF THE DRAWINGS

FIGS. 1A, 1B, and 2C illustrate the various mechanisms of the separation sequences. FIG. 1A depicts action of cleavage sequences, which rely on action by cleavage agent, such as a protease. The cleaving agents act on a translated peptide containing a cleavage agent (i.e., protease) recognition sequence to generate separate peptide products. FIG. 1B shows action of IRES sequences, which act as internal translation initiation sites. Separate translation initiation events occur for the first gene of interest and the second gene of interest, thus resulting in synthesis of separate peptide products. Finally, FIG. 1C shows action of Type 2A sequences, which are believed to cause “ribosome skipping” during the translation process. According to theory, translation of the 2A peptide region results in a failure to form a peptide bond at the junction between the conserved glycine and proline at the carboxy terminus of the 2A peptide. The ribosome continues to translate the downstream segment of the RNA to produce two separate peptide products. Thus, one peptide product retains the 2A peptide region. The use of Type2A sequences in the present invention, however, is not bound or restricted by the inferred mechanistic process by which 2A sequences function.

FIG. 2 shows a set of preferred structures of the retroviral vectors of the present invention. CRU5 is a modified LTR (see Naviaux, R. K. et al. (1996) “The pCL Vector System: Rapid Production of Helper Free, High Titre, Recombinant Retroviruses,” J. Virol. 70: 5701-05); LTR=long terminal repeat; and ψ=packaging signal. All components are cassetted for flexibility. Vector A comprises 5′ (CRU5) and 3′ long terminal repeats (LTR) necessary for replication and integration, ψ packaging signal for packaging into virion particles, promoter (PROM), first gene of interest (GOI₁), separation sequence (SEP SEQ), and second gene of interest (GOI₂). Vector B comprises elements identical to vector B except that the first gene of interest comprises a multiple cloning site (MCS), and the second gene of interest comprises a reporter or selection gene (REP/SEL). Vector C and D are fusion constructs useful for expressing nucleic acids encoding random peptide (RP) candidate agents. Vector C comprises a first gene of interest encoding a random peptide and a second gene of interest comprising a reporter or selection gene. In vector D, the first and second genes of interest express nucleic acids encoding random peptides (RP₁ and RP₂, respectively), thus providing for expression of combinations of random peptides.

FIG. 3 shows a comparison of Type 2A sequences found in aptho- and cardioviral genomes. The general sequence is XXXXXXXXXXLXXDXEXNPGP, where X is any amino acid. Invariant amino acids are shown in bold. Failure of peptide bond formation is believed to occur at the junction between the carboxy terminal glycine and proline (underlined). The 2A sequence also shows a number of residues with conserved amino acid substitutions. Residues at the 2 position are mainly polar amino acids; residues at the 3 position are aliphatic or small amino acids; residues at the 5 position comprises small amino acids; residues at the 6 position are aromatic amino acids; residues at the 7 position are polar amino acids; residues at the 8 position are non-polar amino acids; residues at the 12 position are aliphatic or small aliphatic amino acids; residues at the 13 position are non-polar amino acids; and residues at the 15 position are aliphatic amino acids. Generally, classes of amino acids are defined according to those skilled in the art (see for example, Taylor, W. R. (1986) “The Classification of Amino Acid Conservation,” J. Theor. Biol. 119: 205-18 and U.S. Pat. No. 5,994,306).

FIG. 4A shows the structure of a retroviral vector CRU5-GFP-2A-Puro comprising a fusion nucleic acid expressing separate reporter protein and selection protein. The vector uses the FMDV-2A separation sequence to express separate GFP protein and puromycin transferase (Puro). FIG. 4B is a Western analysis with anti-GFP anti-sera of extracts from Jurkat cells transduced with CRU5-GFP-2A-PURO. The species of GFP detected in cells infected with CRU5-GFP-2A migrates slightly slower than GFP because of additional amino acids contributed by the 2A region. The absence of higher molecular weight GFP species suggests that separation efficiency of the 2A sequence is high. FIG. 4C shows time course of GFP expression of cells infected with CRU-5-GFP-2A-PURO and placed in puromycin selection media (see Experiment 2). With increasing time, the number of cells expressing GFP increases steadily with continued growth in puromycin while the number of cells that do not express GFP decreases. By day seven, 99.9% of surviving cells express GFP, thus demonstrating co-selectability of the GFP reporter and puromycin transferase activities.

FIG. 5A is a photomicrograph of HEK293 cells transduced with CRU5-myrGFPp21 retroviral construct, demonstrating efficient membrane targeting of myrGFP-p21 fusion protein. Identical results were obtained with a CRU5-myrGFP-2A-p21 construct (not shown). FIG. 5B depicts the structure of retroviral vectors used to show efficient production of separate reporter protein and dominant effector protein. The vector CRU5-myrGFP-p21 encodes a fusion protein linking GFP containing an N-myristolation sequence to the p21 cell cycle inhibitor protein. The p21 protein localizes to the nucleus through a bipartite nuclear localization signal present at the carboxy terminus. The CRU5-myrGFP-2A-p21 retroviral construct encodes a fusion protein with an FMDV-2A separation sequence inserted between the coding regions for myrGFP and p21 proteins. FIG. 5C shows the effects of CRU5-myrGFP and CRU5-myrGFP-2A-p21 on the cell cycle as assayed by FACS (Lorens, et al. (2000) Mol. Ther. 1: 438-47) (see Experiment 3). Infected cells were stained with Hoechst 33342 and GFP expressing cells analyzed for DNA content. The CRU5-myrGFP-p21 expressing cells (upper panel) show a cell cycle distribution similar to control infected or non-GFP expressing cells (not shown), thus establishing lack of significant nuclear localization of myrGFP-p21 fusion protein. In contrast, the CRU5-myrGFP-2A-p21 expressing cells (lower panel) show cell cycle arrest at G1, demonstrating separation of myrGFP from p21 and subsequent nuclear localization of p21 protein.

FIG. 6A depicts constructs used to show use of separation sequences to generate separate proteins targeted to distinct cellular compartments and the resulting alteration of a cellular phenotype. The CRU5-Lyt2 is a retroviral construct comprising a fusion nucleic acid encoding mouse Lyt2, a truncated form of the CD8 receptor containing a signal peptide. The CRU5-Lyt2-2A-p21 encodes Lyt2 and p21 proteins, which are separated by a FMDV-2A sequence. FIG. 6B shows the effects of expressing CRU5-Lyt2 and CRU5-Lyt2-2A-p21 in human lung carcinoma cell line, A549. Cells infected with retroviruses were stained will cell tracker dye PKH, incubated for 24 or 72 hrs, stained with anti-Lyt2 antibodies, and then analyzed by FACS (see Experiment 4). Lyt2-expressing and non-expressing cell were gated and correlated with cell tracker PKH fluorescence. For CRU5-Lyt2 infected cells, the Lyt-2 expressing and non-expressing cell populations gave similar cell tracker fluorescence (upper panel). In contrast, CRU5-Lyt2-2A-p21 infected cells gave higher cell tracker fluorescence (lower panel) for the Lyt2 expressing cells relative to Lyt2 non-expressing cells. These results demonstrate that in CRU5-Lyt2-2A-p21 infected cells, the Lyt2 localizes to the cell membrane while the p21 localizes to the nucleus, where it induces a growth arrest phenotype. The data shows that the membrane targeting function of Lyt2 is compatible with the nuclear, cell cycle effects of p21 when derived from a 2A processed polyprotein.

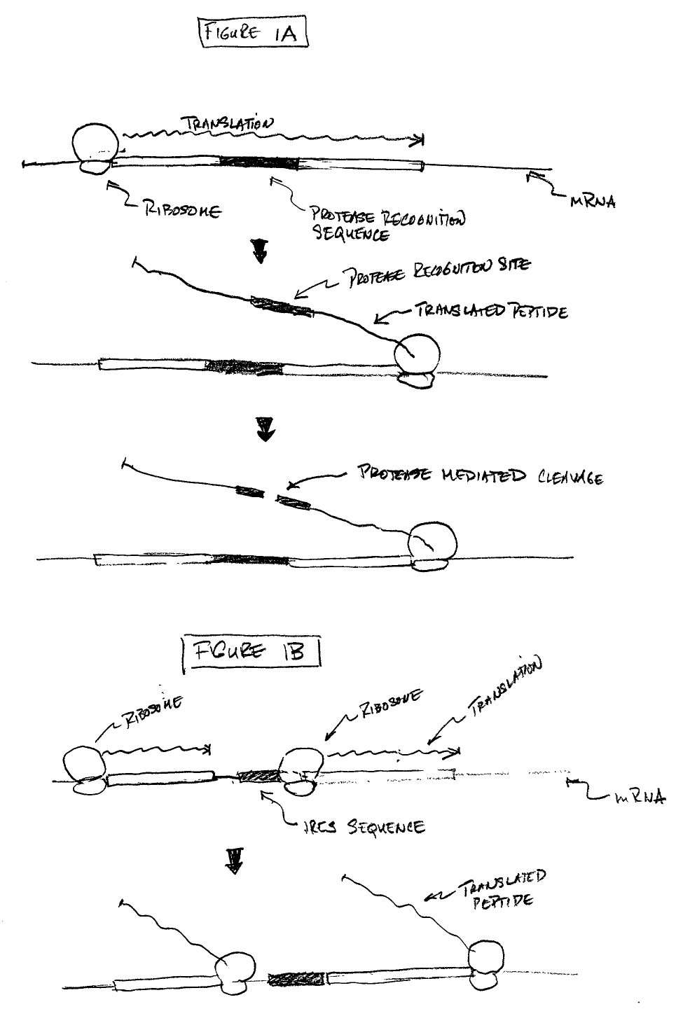

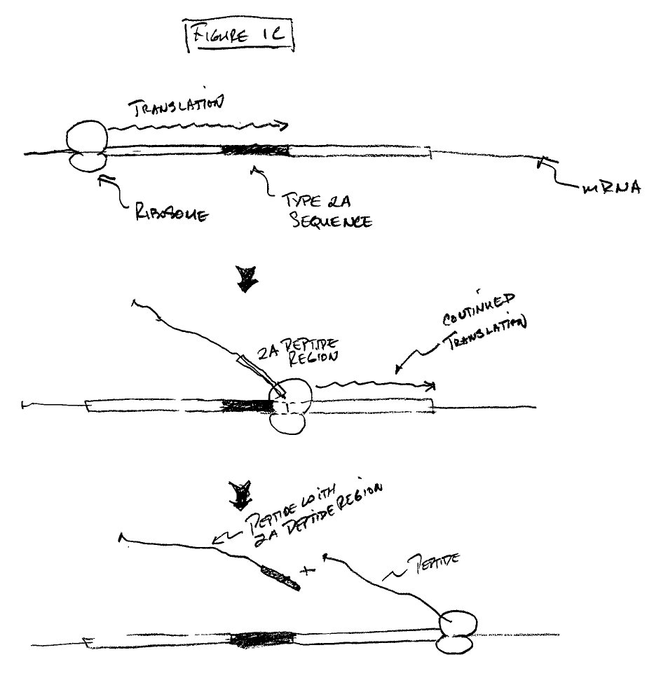

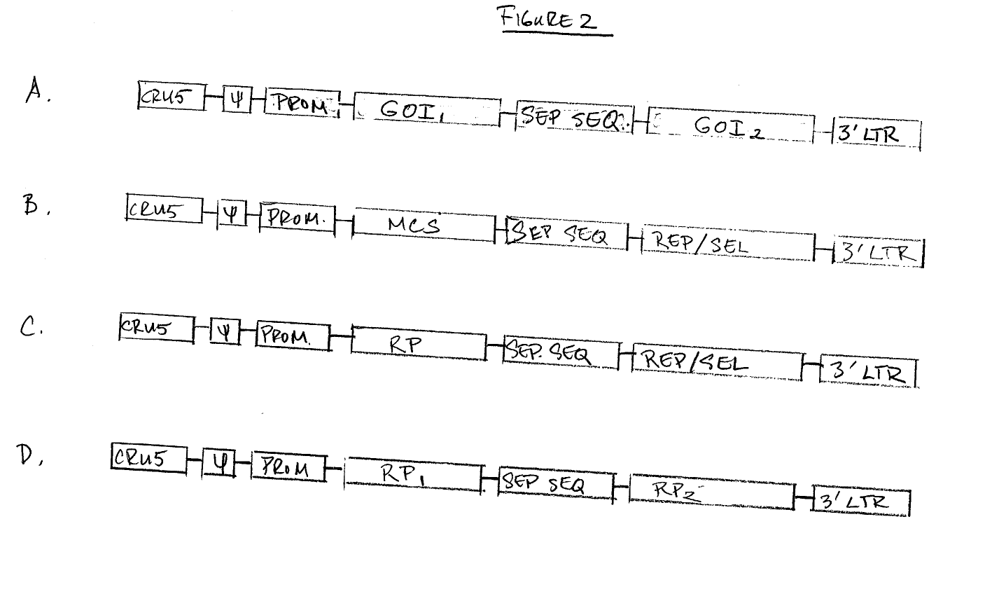

### DETAILED DESCRIPTION OF THE INVENTION

The present invention provides compositions useful for expressing a plurality of genes of interest under the control of a single promoter. By a “plurality” herein is meant at least two or more genes of interest. In particular, the invention provides for compositions to express separate gene products by use of separation sequences acting at the level of RNA or protein. These separation sequences are described in WO 99/58663, which is hereby expressly incorporated by reference.

The present invention relates to vectors comprising fusion nucleic acids comprising a promoter, first gene of interest, separation sequence, and second gene of interest. The vectors may be extrachromosomal vectors that exist either transiently or stably in the cytoplasm or may be vectors that stably integrate into the genome of the host cell. Variety of such vectors for expressing fusion nucleic acids are well known in the art.

In a preferred embodiment, the vectors are retroviral vectors. By “retroviral vectors” herein is meant vectors used to introduce into a host the fusion nucleic acids of the present invention in the form of a RNA viral particle, as is generally outlined in PCT US 97/01019 and PCT US 97/01048, both of which are expressly incorporated by reference. Any number of suitable retroviral vectors may be used.

Preferred retroviral vectors include a vector based on the murine stem cell virus (MSCV) (see Hawley, R. G. et al. (1994) Gene Ther. 1: 136-38) and a modified MFG virus (Riviere, I. et al. (1995) Genetics 92: 6733-37), and pBABE (see PCT US97/01019, incorporated by reference). In addition, particularly well suited retroviral transfection systems for generating retroviral vectors are described in Mann et al., supra; Pear, W. S. et al. (1993) Pro. Natl. Acad. Sci. USA 90: 8392-96; Kitamura, T. et al. (1995) Proc. Natl. Acad. Sci. USA 92: 9146-50; Kinsella, T. M. et al. (1996) Hum. Gene Ther. 7:1405-13; Hofmann, A. et al. (1996) Proc. Natl. Acad. Sci. USA 93: 5185-90; Choate, K. A. et al. (1996) Hum. Gene Ther. 7: 2247-53; WO 94/19478; PCT US97/01019, and references cited therein, all of which are incorporated by reference.

The retroviral vectors of the present invention comprise fusion nucleic acids. By “nucleic acid” or “oligonucleotide” or grammatical equivalents herein is meant at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, S. L. et al. (1993) Tetrahedron 49: 1925-63 and references therein; Letsinger, R. L. et al. (1970) J. Org. Chem. 35: 3800-03; Sprinzl, M. et al. (1977) Eur. J. Biochem. 81: 579-89; Letsinger, R. L. et al. (1986) Nucleic Acids Res. 14: 3487-99; Sawai et al (1984) Chem. Lett. 805; Letsinger, R. L. et al. (1988) J. Am. Chem. Soc. 110: 4470; and Pauwels et al. (1986) Chemica Scripta 26:141-49), phosphorothioate (Mag, M. et al. (1991) Nucleic Acids Res. 19: 1437-41; and U.S. Pat. No. 5,644,048), phosphorodithioate (Briu et al. (1989) J. Am. Chem. Soc. 111: 2321), O-methylphophoroamidite linkages (see Eckstein, Oligonucleotides and Analogues: A Practical Approach, Oxford University Press, 1991), and peptide nucleic acid backbones and linkages (Egholm, M. (1992) Am. Chem. Soc. 114: 1895-97; Meier et al. (1992) Chem. Int. Ed. Engl. 31:1008; Egholm, M (1993) Nature 365: 566-68; Carlsson, C. et al. (1996) Nature 380: 207, all of which are incorporated by reference). Other analog nucleic acids include those with positive backbones (Dempcy, R. O. et al. (1995) Proc. Natl. Acad. Sci. USA 92: 6097-101); non-ionic backbones (U.S. Pat. Nos. 5,386,023, 5,637,684, 5,602,240, 5,216,141 and 4,469,863; Kiedrowshi et al. (1991) Angew. Chem. Intl. Ed. English 30: 423; Letsinger, R. L. et al. (1988) J. Am. Chem. Soc. 110: 4470; Letsinger, R. L. et al. (1994) Nucleoside & Nucleotide 13: 1597; Chapters 2 and 3, ASC Symposium Series 580, “Carbohydrate Modifications in Antisense Research”, Ed. Y. S. Sanghui and P. Dan Cook; Mesmaeker et al. (1994) Bioorganic & Medicinal Chem. Lett. 4: 395; Jeffs et al. (1994) J. Biomolecular NMR 34: 17; (1996) Tetrahedron Left. 37: 743) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al. (1995) Chem. Soc. Rev. 169-176). Several nucleic acid analogs are described in Rawls, C & E News Jun. 2, 1997, page 35. All of these references are hereby expressly incorporated by reference.

The nucleic acids may be single stranded or double stranded, as specified, or contain portions of both double stranded or single stranded sequence. The nucleic acid may be DNA, both genomic and cDNA, RNA or hybrid, where the nucleic acid contains any combination of deoxyribo- and ribonucleotides, and any combination of bases, including uracil, adenine, thymine, cytosine, guanine, xanthine hypoxanthine, isocytosine, isoguanine, etc., although generally occurring bases are preferred. As used herein, the term “nucleoside” includes nucleotides as well as nucleoside and nucleotide analogs, and modified nucleosides such as amino modified nucleosides. In addition, “nucleoside” includes non-naturally occurring analog structures. Thus, for example, the individual units of a peptide nucleic acid, each containing a base, are referred herein as a nucleotide.

By “fusion nucleic acid” herein is meant a plurality of nucleic acid components that are joined together, either directly or indirectly. As will be appreciated by those in the art, in some embodiments the sequences described herein may be DNA, for example when extrachromosomal plasmids are used, or RNA when retroviral vectors are used. In some embodiments, the sequences are directly linked together without any linking sequences while in other embodiments linkers such as restriction endonuclease cloning sites, linkers encoding flexible amino acids, such as glycine or serine linkers such as known in the art, are used, as further discussed below.

The fusion nucleic acids may encode fusion polypeptides. By fusion polypeptide or fusion peptide or grammatical equivalents herein is meant a protein, as defined below, composed of a plurality of protein components that, while typically joined in the native state, are joined by the respective amino and carboxy termini through a peptide linkage to form a continuous polypeptide. Plurality in this context means at least two, and preferred embodiments generally utilize three to twelve components, although more may be used. It will be appreciated that the protein components can be joined directly or joined through a peptide linker/spacer as outlined below.

The fusion nucleic acids of the present invention further comprise a first and a second gene of interest. By “gene of interest” herein is meant a multiple cloning site, as more fully explained below, or any nucleic acid sequence capable of encoding a protein or protein of interest. However, in some embodiments, the gene of interest encompasses a nucleic acid that does not encode a protein, for example antisense nucleic acids, ribozymes, and RNAi molecules (i.e., interfering RNAs). In other embodiments, the gene of interest is a regulatory element, including, but not limited to, promoter/enhancer elements, chromatin organizing sequences, ribosome binding sequences, mRNA splicing sequences, and the like.

By “protein” or “protein of interest” herein is meant at least two covalently attached amino acids, which includes proteins, polypeptides, oligopeptides and peptides. In a preferred embodiment, a protein is made up of naturally occurring amino acids and peptide bonds, such as proteins synthesized by the cellular translation system. However, as used below, a protein may also be made up of synthetic peptidomimetic structures. Thus amino acid or peptide residue as used herein means both naturally occurring and synthetic amino acids. For example, homo-phenylalanine, citrulline, and norleucine are considered amino acids for the purposes of the invention. “Amino acids” also includes imino residues such as proline and hydroxyproline. The side chains may be either the (R) or (S) configuration. In the preferred embodiment, the amino acids are in the (S) or L configuration. If non-naturally occurring side chains are used, non-amino acid substituents may be used for example to prevent or retard in-vivo degradations. Proteins including non-naturally occurring amino acids may be synthesized or in some cases, made by recombinant techniques (see van Hest, J. C. et al. (1998) FEBS Lett. 428: 68-70 and Tang et al. (1999) Abstr. Pap. Am. Chem. S218: U138-U138 Part 2, both of which are expressly incorporated by reference herein).

In one preferred embodiment, the gene of interest comprises a reporter gene. By “reporter gene” or “selection gene” or grammatical equivalents herein is meant a gene that by its presence in a cell (i.e., upon expression) allows the cell to be distinguished from a cell that does not contain the reporter gene. Reporter genes can be classified into several different types, including detection genes, survival genes, death genes, cell cycle genes, cellular biosensors, proteins producing a dominant cellular phenotype, and conditional gene products. In the present invention, expression of the protein product causes the effect distinguishing between cells expressing the reporter gene and those that do not. As is more fully outlined below, additional components, such as substrates, ligands, etc., may be additionally added to allow selection or sorting on the basis of the reporter gene.

In a preferred embodiment, the gene of interest is a reporter gene. In one aspect, the reporter gene encodes a protein that can be used as a direct label, for example a detection gene for sorting the cells or for cell enrichment by FACS. In this embodiment, the protein product of the reporter gene itself can serve to distinguish cells that are expressing the reporter gene. In this embodiment, suitable reporter genes include those encoding green fluorescent protein (GFP, Chalfie, M. et al. (1994) Science 263: 802-05; and EGFP, Clontech—Genbank Accession Number U55762), blue fluorescent protein (BFP, Quantum Biotechnologies, Inc. 1801 de Maisonneuve Blvd. West, 8th Floor, Montreal (Quebec) Canada H3H 1J9; Stauber, R. H. (1998) Biotechniques 24: 462-71; Heim, R. et al. (1996) Curr. Biol. 6: 178-82), enhanced yellow fluorescent protein (EYFP, Clontech Laboratories, Inc., 1020 East Meadow Circle, Palo Alto, Calif. 94303), Anemonia majano fluorescent protein (amFP486, Matz, M. V. (1999) Nat. Biotech. 17: 969-73), Zoanthus fluorescent proteins (zFP506 and zFP538; Matz, supra), Discosoma fluorescent protein (dsFP483, drFP583; Matz, supra), Clavularia fluorescent protein (cFP484; Matz, supra); luciferase (for example, firefly luciferase, Kennedy, H. J. et al. (1999) J. Biol. Chem. 274: 13281-91; Renilla reniformis luciferase, Lorenz, W. W. (1996) J Biolumin. Chemilumin. 11: 31-37; Renilla muelleri luciferase, U.S. Pat. No. 6,232,107); β-galactosidase (Nolan, G. et al. (1988) Proc. Natl. Acad. Sci. USA 85: 2603-07); β-glucouronidase (Jefferson, R. A. et al. (1987) EMBO J. 6: 3901-07; Gallager, S., “GUS Protocols: Using the GUS Gene as a reporter of gene expression,” Academic Press, Inc., 1992); and secreted form of human placental alkaline phosphatase, SEAP (Cullen, B. R. et al. (1992) Methods Enzymol. 216: 362-68). In a preferred embodiment, the codons of the reporter genes are optimized for expression within a particular organism, especially mammals, and particularly for humans (see Zolotukhin, S. et al. (1996) J. Virol. 70: 4646-54; U.S. Pat. 5,968,750; U.S. Pat. No. 6,020,192; all of which are expressly incorporated by reference).

In another embodiment, the reporter gene encodes a protein that will bind a label that can be used as the basis of the cell enrichment (e.g., sorting); that is, the reporter gene serves as an indirect label or detection gene. In this embodiment, the reporter gene preferably encodes a cell-surface protein. For example, the reporter gene may be any cell-surface protein not normally expressed on the surface of the cell, such that secondary binding agents serve to distinguish cells that contain the reporter gene from those that do not. Alternatively, albeit non-preferably, reporters comprising normally expressed cell-surface proteins could be used, and differences between cells containing the reporter construct and those without could be determined. Thus, secondary binding agents bind to the reporter protein. These secondary binding agents are preferably labeled, for example with fluors, and can be antibodies, haptens, etc. For example, fluorescently labeled antibodies to the reporter gene can be used as the label. Similarly, membrane-tethered streptavidin could serve as a reporter gene, and fluorescently-labeled biotin could be used as the label, i.e., the secondary binding agent. Alternatively, the secondary binding agents need not be labeled as long as the secondary binding agent can be used to distinguish the cells containing the construct; for example, the secondary binding agents may be used in a column, and the cells passed through, such that the expression of the reporter gene results in the cell being bound to the column, and a lack of the reporter gene results in the cells not being retained on the column. Other suitable reporter proteins/secondary labels include, but are not limited to, antigens and antibodies, enzymes and substrates (or inhibitors), etc.

In a preferred embodiment, the reporter gene comprises a survival gene that serves to provide a nucleic acid without which the cell cannot survive, such as drug resistance genes. In this embodiment, expressing the survival gene allows selection of cells expressing the fusion nucleic acid by identifying cells that survive, for example in presence of a selection drug. Examples of drug resistance genes include, but are not limited to, puromycin resistance gene (puromycin-N-acetyl-transferase) (de la Luna, S. et al. (1992) Methods Enzymol. 216: 376-85), G418 neomycin resistance gene, hygromycin resistance gene (hph), and blasticidine resistance genes (bsr, brs, and BSD; Pere-Gonzalez, et al.(1990) Gene, 86: 129-34; Izumi, M. et al. (1991) Exp. Cell Res. 197: 229-33; Itaya, M. et al. (1990) J. Biochem. 107:799-801; Kimura, M. et al. (1994) Mol. Gen. Genet. 242: 121-29). In addition, generally applicable survival genes are the family of ATP-binding cassette transporters, including multiple drug resistance gene (MDR1) (see Kane, S. E. et. al. (1988) Mol. Cell. Biol. 8: 3316-21 and Choi, K. H. et al. (1988) Cell 53: 519-29), multidrug resistance associated proteins (MRP) (Bera, T. K. et al. (2001) Mol. Med. 7: 509-16), and breast cancer associated protein (BCRP or MXR) (Tan, B. et al. (2000) Curr. Opin. Oncol. 12: 450-58). When expressed in cells, these selectable genes can confer resistance to a variety of toxic reagents, especially anti-cancer drugs (i.e. methotrexate, colchicine, tamoxifen, mitoxanthrone, and doxorubicin). As will be appreciated by those in the art, the choice of the selection/survival gene will depend on the host cell type used.

In a preferred embodiment, the reporter gene comprises a death gene that causes the cells to die when expressed. Death genes fall into two basic categories: death genes that encode death proteins requiring a death ligand to kill the cells, and death genes that encode death proteins that kill cells as a result of high expression within the cell and do not require the addition of any death ligand. Preferred are cell death mechanisms that requires a two-step process: the expression of the death gene and induction of the death phenotype with a signal or ligand such that the cells may be grown expressing the death gene, and then induced to die. A number of death genes/ligand pairs are known, including, but not limited to, the Fas receptor and Fas ligand (Schneider, P. et al. (1997) J. Biol. Chem. 272: 18827-33; Gonzalez-Cuadrado, S. et al. (1997) Kidney Int. 51: 1739-46; Muruve, D. A. et al. (1997) Hum. Gene Ther. 8: 955-63); p450 and cyclophosphamide (Chen, L. et al. (1997) Cancer Res. 57: 4830-37); thymidine kinase and gangcylovir (Stone, R. (1992) Science 256: 1513); tumor necrosis factor (TNF) receptor and TNF; and diptheria toxin and heparin-binding epidermal growth factor-like growth factor (HBEGF) (see WO 01/34806, hereby incorporated by reference). Alternatively, the death gene need not require a ligand, and death results from high expression of the gene; for example, the overexpression of a number of programmed cell death (PCD) proteins known to cause cell death, including, but not limited to, caspases, bax, TRADD, FADD, SCK, MEK, etc.

In a preferred embodiment, death genes also include toxins that cause cell death, or impair cell survival or cell function when expressed by a cell. These toxins generally do not require addition of a ligand to produce toxicity. An example of a suitable toxin is campylobacter toxin CDT (Lara-Tejero, M. (2000) Science, 290: 354-57). Expression of the CdtB subunit, which has homology to nucleases, causes cell cycle arrest and ultimately cell death. Another toxin, the diptheria toxin (and similar Pseudomonas exotoxin), functions by ADP ribosylating ef-2 (elongation factor 2) molecule in the cell and preventing translation. Expression of the diptheria toxin A subunit induces cell death in cells expressing the toxin fragment. Other useful toxins include cholera toxin and pertussis toxin (catalytic subunit-A ADP ribosylates the G protein regulating adenylate cyclase), pierisin from cabbage butterflys (induces apoptosis in mammalian cells; Watanabe, M. (1999) Proc. Natl. Acad. Sci. USA 96: 10608-13), phospholipase snake venom toxins (Diaz, C. et al. (2001) Arch. Biochem. Biophys. 391: 56-64), ribosome inactivating toxins (e.g., ricin A chain, Gluck, A. et al. (1992) J. Mol. Biol. 226: 411-24;and nigrin, Munoz, R. et al. (2001) Cancer Lett. 167: 163-69), and pore forming toxins (hemolysin and leukocidin). When the cells are neuronal cells, neuronal specific toxins may be used to inhibit specific neuronal functions. These include bacterial toxins such as botulinum toxin and tetanus toxin, which are proteases that act on synaptic vesicle associated proteins (i.e., synaptobrevin) to prevent neurotransmitter release (see Binz, T. et al. (1994) J. Biol. Chem. 269: 9153-58; Lacy, D. B. et al. (1998) Curr. Opin. Struct. Biol. 8: 778-84).

Another preferred embodiment of a reporter molecule comprises a cell cycle gene, that is, a gene that causes alterations in the cell cycle. For example, Cdk interacting protein p21 (see Harper, J. W. et al. (1993) Cell 75: 805-16), which inhibits cyclin dependent kinases, does not cause cell death but causes cell-cycle arrest. Thus, expressing p21 allows selecting for regulators of promoter activity or regulators of p21 activity based on detecting cells that grow out much more quickly due to low p21 activity, either through inhibiting promoter activity or inactivation of p21 protein activity. As will be appreciated by those in the art, it is also possible to configure the system to select cells based on their inability to grow out due to increased p21 activity. Similar mitotic inhibitors include p27, p57, p16, p15, p18 and p19, p19 ARF (human homolog p14 ARF). Other cell cycle proteins useful for altering cell cycle include cyclins (Cln), cyclin dependent kinases (Cdk), cell cycle checkpoint proteins (e.g., Rad17, p53), Cks1 p9, Cdc phosphatases (e.g., Cdc 25) etc.

In yet another preferred embodiment, the gene of interest comprises a nucleic acid encoding a cellular biosensor. By a “cellular biosensor” herein is meant a gene product that when expressed within a cell can provide information about a particular cellular state. Biosensor proteins allow rapid determination of changing cellular conditions, for example Ca⁺² levels in the cell, pH within cellular organelles, and membrane potentials (see Miesenbock, G. et al. (1998) Nature 394: 192-95). An example of an intracellular biosensor is Aequorin, which emits light upon binding to Ca⁺² ions. The intensity of light emitted depends on the Ca⁺² concentration, thus allowing measurement of transient calcium concentrations within the cell. When directed to particular cellular organelles by fusion partners, as more fully described below, the light emitted by Aequorin provides information about Ca⁺² concentrations within the particular organelle. Other intracellular biosensors are chimeric GFP molecules engineered for fluorescence resonance energy transfer (FRET) upon binding of an analyte, such as Ca⁺² (Miyawaki, A. et al. (1997) Nature 388: 882-87; Miyakawa, A. et al. (1997) Mol. Cell. Biol. 8: 2659-76). For example, Camelot consists of blue or cyan mutant of GFP, calmodulin, CaM binding domain of myosin light chain kinase, and a green or yellow GFP. Upon binding of Ca⁺² by the CaM domain, FRET occurs between the two GFPs because of a structural change in the chimera. Thus, FRET intensity is dependent on the Ca⁺² levels within the cell or organelle (Kerr, R. et al. Neuron (2000) 26: 583-94). Other examples of intracellular biosensors include sensors for detecting changes in cell membrane potential (Siegel, M. et al. (1997) Neuron 19: 735-41; Sakai, R. (2001) Eur. J. Neurosci. 13: 2314-18), monitoring exocytosis (Miesenbrock, G. et al. (1997) Proc. Natl. Acad. Sci. USA 94: 3402-07), and measuring intracellular/organellar ATP concentrations via luciferase protein (Kennedy, H. J. et al. (1999) J. Biol. Chem. 274: 13281-91). These biosensors find use in monitoring the effects of various cellular effectors, for example pharmacological agents that modulate ion channel activity, neurotransmitter release, ion fluxes within the cell, and changes in ATP metabolism.

Other intracellulular biosensors comprise detectable gene products with sequences that are responsive to changes in intracellular signals. These sequences include peptide sequences acting as substrates for protein kinases, peptides with binding regions for second messengers, and protein interaction sequences sensitive to intracellular signaling events (see for example, U.S. Pat. No. 5,958,713 and U.S. Pat. No. 5,925,558). For example, a fusion protein construct comprising a GFP and a protein kinase recognition site allows measuring intracellular protein kinase activity by measuring changes in GFP fluorescence arising from phosphorylation of the fusion construct. Alternatively, the GFP is fused to a protein interaction domain whose interaction with cellular components are altered by cellular signaling events. For example, it is well known that inositol-triphosphate (InsP3) induces release of Ca⁺² from intracellular stores into the cytoplasm, which results in activation of a kinases responsible for regulating various cellular responses. The precursor to InsP3 is phosphatidyl-inositol-4,5-bisphosphate (PtdInsP₂), which is localized in the plasma membrane and cleaved by phospholipase C (PLC) following activation of an appropriate receptor. Many signaling enzymes are sequestered in the plasma membrane through pleckstrin homology domains that bind specifically to PtdInsP₂. Following cleavage of PtdInsP₂, the signaling proteins translocate from the plasma membrane into the cytosol where they activate various cellular pathways. Thus, a reporter molecule such as GFP fused to a pleckstrin domain will act as a intracellular sensor for phospholipase C activation (see Haugh, J. M. et al. (2000) J. Cell. Biol. 15: 1269-80; Jacobs, A. R. et al. (2001) J. Biol. Chem. 276: 40795-802; and Wang, D. S. et al. (1996) Biochem. Biophys. Res. Commun. 225: 420-26). Other similar constructs are useful for monitoring activation of other signaling cascades and applicable as assays in screens for candidate agents that inhibit or activate particular signaling pathways.

Since protein interaction domains, such as the described pleckstrin homology domain, are important mediators of cellular responses and biochemical processes, other preferred genes of interest are proteins containing protein-interaction domains. By protein-interaction domain herein is meant a polypeptide region that interacts with other biomolecules, including other proteins, nucleic acids, lipids etc. These protein domains frequently act to provide regions that induce formation of specific multiprotein complexes for recruiting and confining proteins to appropriate cellular locations or affect specificity of interaction with targets ligands, such as protein kinases and their substrates. Thus, many of these protein domains are found in signaling proteins. Protein-interaction domains comprise modules or micro-domains ranging about 20-150 amino acids that can be expressed in isolation and bind to their physiological partners. Many different interaction domains are known, most of which fall into classes related by sequence or ligand binding properties. Accordingly, the genes of interest comprising interaction domains may comprise proteins that are members of these classes of protein domains and their relevant binding partners. These domains include, among others, SH2 domains (src homology domain 2), SH3 domain (src homology domain 3), PTB domain (phosphotyrosine binding domain), FHA domain (forkedhead associated domain), WW domain, 14-3-3 domain, pleckstrin homology domain, C1 domain, C2 domain, FYVE domain (Fab-1, YGL023, Vps27, and EEA1), death domain, death effector domain, caspase recruitment domain, Bcl-2 homology domain, bromo domain, chromatin organization modifier domain, F box domain, hect domain, ring domain (Zn⁺² finger binding domain), PDZ domain (PSD-95, discs large, and zona occludens domain), sterile a motif domain, ankyrin domain, arm domain (armadillo repeat motif), WD 40 domain and EF-hand (calretinin), PUB domain (Suzuki T. et al. (2001) Biochem. Biophys. Res. Commun. 287: 1083-87), nucleotide binding domain, Y Box binding domain, H. G. domain, all of which are well known in the art. Since protein interactions domains are pervasive in cellular signal transduction cascades and other cellular processes, such as cell cycle regulation and protein degradation, expression of single proteins or multiple proteins with interaction domains acting in specific signaling or regulatory pathway may provide a basis for inactivating, activating, or modulating such pathways in normal and diseased cells. In another aspect, the preferred embodiments comprise binding partners of these interactions domains, which are well known to those skilled in the art or are identifiable by well known methods (e.g., yeast two hybrid technique, co-precipitation of immune complexes etc.).

Included within the protein-interaction domains are transcriptional activation domains capable of activating transcription when fused to an appropriate DNA binding domain. Transcriptional activation domains are well known in the art. These include activator domains from GAL4 (amino acids 1-147; Fields, S. et al. (1989) Nature 340: 245-46; Gill, G. et al. (1990) Proc. Natl. Acad. Sci. USA 87: 2127-31), GCN4 (Hope, I. A. et al. (1986) Cell 46: 885-94), ARD1 (Thukral, S. K. et al. (1989) Mol. Cell. Biol. 9: 2360-69), human estrogen receptor (Kumar, V. et al. (1987) Cell 51: 941-51), VP16 (Triezenberg, S. J. et al. (1988) Genes Dev. 2: 718-29), Sp1 (Courey, A. J. (1988) Cell 55: 887-98), AP-2 (Williams, T. et al. (1991) Genes Dev. 5: 670-82), and NF-kB p65 subunit and related Rel proteins (Moore, P. A. et al. (1993) Mol. Cell. Biol. 13: 1666-74). DNA binding domains include, among others, leucine zipper domain, homeo box domain, Zn⁺² finger domain, paired domain, LIM domain, ETS domain, and T Box domain. Since the genes of interest may comprise DNA binding domains and transcriptional activation domains, other genes of interest useful for expression in the present invention are transcription factors. Preferred transcription factors are those producing a cellular phenotype when expressed within a particular cell type. As not all cells will respond to expression of a particular transcription factor, those skilled in the art can choose appropriate cell strains in which expression of a transcription factor results in dominant or altered phenotypes as described below.

In another preferred embodiment, the gene of interest comprises a nucleic acid encoding a protein whose expression has a dominant effect on the cell. That is, expression of the gene of interest produces an altered cellular phenotype. By “dominant effect” herein is meant that the protein or peptide produces an effect upon the cell in which it is expressed and is detected by the methods described below. The dominant effect may act directly on the cell to produce the phenotype or act indirectly on a second molecule, which leads to a specific phenotype. Dominant effect is produced by introducing small molecule effectors, expressing a single protein, or by expressing multiple proteins acting in combination (i.e., synergistically on a cellular pathway or multisubunit protein effectors). As is well known in the art, expression of a variety of genes of interest may produce a dominant effect. Expressed proteins may be mutant proteins that are constitutive for a catalytic activity (Segouffin-Cariou, C. et al. (2000) J. Biol. Chem. 275: 3568-76; Luo et al. (1997) Mol. Cell. Biol. 17: 1562-71) or are inactive forms that sequester or inhibit activity of normal binding partners (Bossu, P. (2000) Oncogene, 19: 2147-54; Mochizuki, H. (2001) Proc. Natl Acad. Sci. USA 98: 10918-23). The inactive forms as defined herein include expression of small modular protein-interaction regions or other domains that bind to binding partners in the cell (see for example, Gilchrist, A. et al. (1999) J. Biol. Chem. 274: 6610-16). Dominant effects are also produced by overexpression of normal cellular proteins, expression of proteins not normally expressed in a particular cell type, or expression of normally functioning proteins in cells lacking functional proteins due to mutations or deletions (Takihara, Y. et al. (2000) Carcinogenesis 21: 2073-77; Kaplan, J. B. (1994) Oncol. Res. 6: 611-15). Random peptides or biased random peptides introduced into cells can also produce dominant effects. An exemplary effect of a dominant effect by a peptide is random peptides which bind to Src SH3 domain resulting in increased Src activity due to the peptides' antagonistic effect on negative regulation of Src (see Sparks, A. B. et al. (1994) J Biol Chem. 269: 23853-56).

As defined herein, dominant effect is not restricted to the effect of the protein on the cell expressing the protein. A dominant effect may be on a cell contacting the expressing cell or by secretion of the protein encoded by the gene of interest into the cellular medium. Proteins with dominant effect on other cells are conveniently directed to the plasma membrane or secretion by incorporating appropriate secretion and membrane localization signals. These membrane bound or secreted dominant effector proteins may comprise cytokines and chemokines, growth factors, toxins, extracellular proteases, cell surface receptor ligands (e.g., sevenless type receptor ligands), and adhesion proteins (e.g., L1, cadherins, integrins, laminin, etc.).

In an alternative embodiment, the gene of interest comprises a nucleic acid encoding a conditional gene product. By conditional gene product herein is meant a gene product whose activity is only apparent under certain conditions, for example at particular ranges of temperature. Other factors that conditionally affect activity of a protein include, but are not limited to, ion concentration, pH, and light (see Hager, A. (1996) Planta 198: 294-99; Pavelka J. (2001) Bioelectromagnetics 22: 371-83). A conditional gene product produces a specific cellular phenotype under a restrictive condition. In contrast, the conditional gene product does not produce a specific phenotype under permissive conditions. Methods for making or isolating conditional gene products are well known (see for example, White, D. W. et al. (1993) J. Virol. 67:6876-81; Parini, M. C. (1999) Chem. Biol. 6: 679-87)

As is appreciated by those skilled in the art, conditional gene products are useful in examining genes that are detrimental to a cell's survival or in examining cellular biochemical and regulatory pathways in which the gene product functions. For those gene products that affect cell survival, use of conditional gene products allows survival of the cells under permissive conditions, but results in lethality or detriment at the restrictive condition. This feature permits screens at the restrictive condition for candidate agents, such as proteins and small molecules, which may directly or indirectly suppress the effect of conditional gene product but permit maintenance and growth of cells under permissive conditions. In addition, conditional gene products are also useful in screens for regulators of cell physiology when it is also a participant in a cellular regulatory pathway. At the restrictive condition, the conditional gene product ceases to function or becomes activated, resulting in an altered cell phenotype due to dysregulation of the regulatory pathway. Candidate agents are then screened for their ability to activate or inhibit downstream pathways to bypass the disrupted regulatory point. Conditional gene products are well known in the art and include, among others, proteins such dynamin involved in endocytic pathway (Damke, H. et al. (1995) Methods Enzymol. 257: 209-20), p53 involved in tumor suppression (Pochampally, R. et al. (2000) Biochem. Biophys. Res. Comm. 279: 1001-10 and Buckbinder, L. et al. (1994) Proc. Natl. Acad. Sci. USA 91: 10640-44), Vac1 involved in vesicle sorting, proteins involved in viral pathogenesis (SV40 Large T Antigen; Robinson C. C. (1980). J Virol. 35: 246-48), and gene products involved in regulating the cell cycle, such as ubiquitin conjugating enzyme CDC 34 (Ellison, K. S. et al. (1991) J. Biol. Chem. 266: 24116-20).

In another preferred embodiment, the gene of interest comprises cDNA. As more fully explained below, cDNA may be derived from any number of cell types including cDNAs generated from eukaryotic and prokaryotic cells, viruses, cells infected with viruses, pathogens or from genetically altered cells. The cDNA may be a cDNA fragment encoding only a portion of the gene of interest or may encode the entire full length coding region. The cDNA may encode specific domains, such as signaling domains, protein interaction domains (as discussed above), membrane binding domains, targeting domains, nuclear localization domains etc. Furthermore, the cDNA fragment may be “frame shifted” by adding or deleting nucleotides, which may result in an out-of-frame construct, such that pseudorandom peptide or protein is encoded. In addition, the cDNA libraries contemplate various subtracted cDNA libraries or enriched cDNA libraries (e.g., for secreted or membrane proteins; see Kopczynski, C. C. (1998) Proc. Natl. Acad. Sci. USA 95: 9973-78). That is, a cDNA library may be a “complete” cDNA library from a cell, a partial library, an enriched library from one or more cell types or a constructed library with certain cDNAs being removed to form a library.

In a further preferred embodiment, the gene of interest comprises genomic DNA. As elaborated above for cDNA, the genomic DNA can be derived from any number of different cells, including genomic DNA of eukaryotic or prokaryotic cells. They may be from normal cells or cells defective in cellular processes, such as tumor suppression, cell cycle control, or cell surface adhesion. As more fully explained below the genomic DNA may be from entire genomic constructs or fractionated constructs, including random or targeted fractionation.

In a preferred embodiment, the gene of interest comprises a nucleic acid encoding a random peptide sequence of a random peptide library. Generally, nucleic acids encoding peptides ranging from about 4 amino acids in length to about 100 amino acids may be used, with peptides ranging from about 5 to about 50 being preferred, with from about 8 to about 30 being particularly preferred and from about 10 to about 25 being especially preferred. As more fully explained below, the encoded peptides sequences are fully randomized or they are biased in their randomization. Preferred are random peptides linked to a fusion polypeptide. Random peptides expressed by the fusion nucleic acid may be screened for activity against a gene of interest also expressed on the fusion nucleic acid, or the peptide is screened for its ability to produce a dominant or altered phenotype. In one aspect, the expressed random peptide is not linked to fusion partner, but in a more preferred embodiment, the peptide is linked to a fusion partner to structurally constrain the peptide and allow proper interaction with other molecules, as explained more fully below.

As one aspect of the present invention is to express a plurality of separate protein products, a preferred embodiment of the fusion nucleic acids comprises a first gene of interest and a second gene of interest. By a “plurality” of separate protein products herein is meant at least two separate protein products, with each protein product being encoded by a gene of interest.

In one embodiment, the first and second gene of interest comprise the same gene. These constructs allow increased expression of the encoded protein product since two copies of the same gene of interest are expressed in a single transcriptional event. Synthesizing high levels of encoded protein is desirable when needed to produce a cellular phenotype (i.e., dominant or altered phenotype) through maintaining elevated cellular levels of an effector protein, or in industrial applications where maximizing production of a gene of interest is needed to increase efficiency and lower manufacturing costs. Similarly, for example when screening for promoter regulators, signal amplification may be accomplished using two identical reporter genes such as GFP.

In a more preferred embodiment, the first gene of interest is non-identical to the second gene of interest. Thus, the first gene of interest and the second gene of interest may have different nucleic acid sequences, which may manifest itself as differences in amino acid sequence, protein size, protein activities, or protein localization. Since expressing multiple gene products have utility in many different biological, diagnostic, and medical applications, the present invention envisions numerous combinations of first and second genes of interest. Those skilled in the art can choose the combinations most relevant to their needs.

Accordingly, in one preferred embodiment, at least one of the genes of interest encodes a reporter protein. Thus, in one aspect, the first or second gene of interest comprises a reporter gene. The presence of a separation sequence results in synthesis of separate a protein of interest and a reporter protein, which allows detecting expression of the gene of interest by monitoring coexpression of the reporter protein. Producing separate protein of interest and reporter protein obviates any detrimental effect that might arise from fusing a protein of interest to the reporter protein. Additionally, expressing separate reporter protein and protein of interest allows targeting of individual proteins to distinct cellular locations. In some situations, the reporter protein is also an indicator of cellular phenotype, which allows detecting the cell expressing the fusion nucleic acid, but also provides information about the physiological state of the cell.

In another aspect, at least one of the genes of interest comprises a selection gene. Thus, in one aspect, the first or second gene of interest comprises a selection gene. Expression of the gene of interest and a selection gene permits selecting for cells expressing both the gene of interest and the selection gene, for example, a puromycin resistance gene. The presence of separation sequence produces separate protein products of the gene of interest and selection gene, which is important for the reasons described above. If the selection gene is either survival or death gene, expressing various genes of interest is useful in screening for agents that counteract or regulate the action of survival genes in the cell.

In another aspect, at least one of the genes of interest encodes a protein producing a dominant effect on a cell. Thus, in one aspect, the first or second gene of interest comprises a nucleic acid encoding a dominant effector protein. As described above, dominant effect is produced by variety of ways. The protein of interest may be overexpressed natural proteins or expressed mutants or variants of analogs of the natural protein. Classes of proteins producing a dominant effect include signal transduction proteins, protein-interaction domains, cell cycle regulatory proteins, or transcription factors whose expression produces a detectable phenotype in a cell. The expressed protein of interest is active in producing the dominant effect or is active conditionally, requiring a restrictive condition to produce the cellular phenotype. Fusion nucleic acids where at least one of the gene of interest encodes a protein having a dominant effect provides a basis for screening for candidate agents inhibiting or enhancing the dominant effect.

In another preferred embodiment, at least one of the genes of interest comprises a cDNA. Thus, in one aspect, the first or second gene of interest comprises a cDNA. As more fully explained below, the cDNA may be a fragment of a cDNA or a cDNA encoding all the amino acids of the gene from which the cDNA is derived. Expression of fusion nucleic acids where the first gene of interest is a cDNA and a second gene of interest is a reporter gene allows selection of cells expressing the protein product encoded by the cDNA. Alternatively, if the second gene of interest encodes a protein that produces a dominant effect, expression of a variety of cDNAs from a cDNA library will permit screening of cDNA products acting as effectors of the dominantly active protein. By “effectors” herein is meant inhibition, activation or modulation of activity of the protein encoded by second gene of interest. For example, the dominantly acting protein may have tyrosine kinase activity which activates or inhibits signaling cascades to produce a detectable cellular phenotype. Expression of cDNAs encoding proteins that are inhibitors or activators of the kinase activity can suppress the dominant effect of the second gene of interest.

In yet another embodiment, at least one of the genes of interest comprises a nucleic acid encoding a random peptide sequence. Thus, the first or second gene of interest comprises a nucleic acid encoding a random peptide. The peptides may be totally random or biased. The second gene of interest may be any gene of interest described above. For example, the presence of a reporter gene allows monitoring the expression of the random peptide without interfering with the structure or activity of the random peptide. In one aspect, the reporter protein is an indicator of the phenotype of the cell. In another embodiment, the second gene of interest encodes a protein that produces a specific cellular phenotype in the cell, for example by expression of death genes, survival genes, dominant effect proteins, signal transduction proteins, cell cycle regulators, oncogenes, etc. Co-expression of the random peptide will permit screening of candidate peptides capable of increasing, decreasing, or modulating the activity or effect of the encoded second gene of interest. Use of specific separation sequences, such as the Type 2A sequences, provides the ability to co-express the random peptide gene and the gene of interest at similar levels, thus increasing the probability of detecting peptide effectors. Consequently, a gene of interest comprising random peptide also comprises a candidate agent that is screened for effects on a cellular phenotype, as more fully explained below.

As the present invention allows for various combinations of first gene of interest and second gene of interest, one preferred combination comprises a first and second gene of interest encoding different reporter proteins. These constructs provide two different basis for detecting a cell expressing the fusion nucleic acid. For example, the first gene of interest may be a GFP and the second gene of interest a β-galactosidase, which permits increased discrimination of cells expressing the fusion nucleic acid by detecting both GFP and β-galactosidase activities. Alternatively, another combination comprises a first gene of interest comprising a reporter gene and a second gene of interest comprising a selection gene. This allows selection for cells expressing fusion nucleic acid by their expression of the selection gene, such as a drug resistance gene, and expression of the reporter construct.

Another preferred combination comprises a fusion nucleic acid where the first gene of interest encodes a first selection gene and the second gene of interest encodes a second selection gene. Thus, one embodiment of the fusion nucleic acid may comprise a first gene of interest encoding a first multidrug resistance gene (e.g., MDR-1) and a second gene of interest encoding a second multidrug resistance gene (e.g., MRP). Both the MDR-1 and MRP are ATP cassetted transporters implicated in development of cellular tolerance to toxic drugs, especially anti-cancer agents. Expression of these multiple resistance transporters in cancerous cells can limit the effectiveness of chemotherapy. Accordingly, expressing several different multidrug resistance genes allows screening for candidate agents or combination of candidate agents (drug cocktails) effective in inhibiting, directly or indirectly, the activity of multiple drug resistance genes.

In another embodiment, a preferred combination is a first gene of interest encoding a first death gene and the second gene of interest encodes a second death gene. Particularly preferred are death genes involved in a particular death pathway, such as caspase proteases involved in apoptotic pathways and apoptosis related gene Apaf-1 (Cecconi, F. (1999) Cell Death Differ. 6: 1087-98). In some embodiments, expression of one death gene may be insufficient to produce a cell death phenotype, and thus require expression of multiple death related genes. Accordingly, expression of multiple death gene are used to produce a cell death phenotype, for example by expression of Fas and Fas binding protein FADD (Chang, H. Y. et al. (1999) Proc. Natl. Acad. Sci. USA 96: 1252-56).

In another embodiment, the first gene of interest comprises a first biosensor and the second gene of interest comprises a second biosensor. Use of different biosensors permit monitoring of more than one intracellular event. For example, the first gene of interest may comprise an Aequorin Ca⁺² sensor protein while the second is a distinguishable pleckstrin homology-GFP fusion protein, such as pleckstrin-EGFP. This allows simultaneous monitoring of intracellular Ca⁺² and receptor mediated phospholipase C signaling activation, which may be useful in identifying cellular targets involved in regulating the IP3 signaling pathway and for screening candidate agents that act on specific steps of the IP3 signaling process.

Similarly, another preferred combination is a first gene of interest encoding a first dominant effector protein and a second gene of interest encoding a second dominant effector protein. Particularly preferred are dominant effectors acting synergistically or acting in combination to produce a cellular phenotype. One example is coexpression of GAP and Ras to produce transformed phenotype in cells (see Clark G. J. et al. (1997) J. Biol. Chem. 272: 1677-81). The GAP protein appears to contribute to Ras transforming activity by activating the GTPase activity of Ras. By expressing both GAP and Ras in the same cell, the oncogenic potential by the Ras pathway is elevated.

The preferred embodiments also encompass fusion nucleic acids where both the first and second genes of interest encode random peptide candidate agents capable of producing a specific cellular phenotype. The random peptide sequences are preferably members of a library of nucleic acids encoding random peptides, as described below. Expressing multiple random peptides within the same cell provides several advantages, such as the ability to generate novel combinations of random peptides producing a specific cellular phenotype and more efficient screening of peptide candidate agents. Similarly, expression of combinations of genes of interest comprising cDNA or genomic DNA are also contemplated for producing novel combinations of peptides capable of producing an altered cellular phenotype.

In the present invention, there is no particular order of the first and second gene of interest on the fusion nucleic acid. One embodiment may have the first gene of interest upstream of the second gene of interest. Another embodiment may have the second gene of interest upstream and the first gene of interest downstream. By “upstream” and “downstream” herein is meant the proximity to the point of transcription initiation, which is generally localized 5′ to the coding sequence of the fusion nucleic acid. Thus, in a preferred embodiment, the upstream gene of interest is more proximal to the transcription initiation site than the downstream gene of interest.

As will be appreciated by those skilled in the art, the positioning of the first gene of interest relative to the second gene of interest is determined by the person skilled in the art. Factors to consider include the need for detecting expression of a gene of interest, optimizing the levels of synthesis of the protein of interest, and targeting of the proteins to subcellular compartments. In the embodiments described above, where at least one of the genes of interest is a reporter gene, the reporter gene may be placed downstream of the gene of interest so that expression of the reporter gene will be a faithful indication of expression of the gene of interest. This will depend on the types of separation sites chosen by the person skilled in the art. When protease cleavage or Type 2A separation sequences are incorporated into the fusion nucleic acid, a reporter gene situated downstream of the gene of interest will generally provide direct information on expression of the upstream gene of interest. In the case of IRES sequences, however, detecting expression of the reporter protein to monitor expression of an upstream gene of interest is less direct since separate translation initiations occur for the first and second genes of interest, generally resulting in a lower expression of the downstream gene of interest that is regulated by the IRES sequence. In some cases, the ratio of the expressed levels of proteins encoded by the first and second genes of interest when using IRES sequences can be as high as 10:1.

The order of genes of interest on the fusion nucleic acid and the choice of separation sequence is also important when the relative amounts of first and second gene products of interest are at issue. For example, use of IRES sequences may result in lower amounts of downstream gene product as compared to upstream gene product because of differing translation initiation rates. Relative levels of translation initiation is easily determined by comparing expression of upstream gene of interest versus downstream gene of interest. Where controlling expression levels is important, the person skilled in the art will order the gene product needed at higher levels upstream of the downstream gene product when an IRES separation sequence is used. Alternatively, multiple copies of IRES sequences are adaptable to increase expression of the downstream gene of interest. On the other hand, use of protease or Type 2A separation sequences will lessen the need for ordering the genes of interest since these separation sequences tend to produce equal levels of upstream and downstream gene product.

When the genes of interest are targeted to different cellular compartments, targeting and localization sequences are appropriately positioned to direct the separate proteins to their desired locales, as further described below. For example, when directing one protein to the plasma membrane and another protein to the cell nucleus, one preferred embodiment comprises a signal sequence incorporated into the upstream first gene of interest and a nuclear localization sequence incorporated into the downstream second gene of interest. Targeting sequences are appropriately placed to minimize interference with the cellular machinery responsible for directing proteins to various cellular locations.

As the object of the present invention is to produce separate proteins of interest encoded by the first and second gene of interest, the fusion nucleic acids of the present invention incorporates separation sequences. By a “separation sequence” or “separation site” or grammatical equivalents as used herein is meant a sequence that results in protein products not linked by a peptide bond. Separation may occur at the RNA or protein level. By being separate does not preclude the possibility that the protein products of the first and the second gene of interest interact, either non-covalently or covalently, following their synthesis. Thus, the separate protein products may interact through hydrophobic domains, protein-interaction domains, commonly bound ligands, or through formation of disulfide linkages between the proteins.

In the present invention, various types of separation sequences may be employed. In one preferred embodiment, the separation sequence comprises a nucleic acid encoding a recognition site for a protease. A protease recognizing the site cleaves the translated protein product into two or more peptides. Preferred protease cleavage sites and cognate proteases include, but are not limited to, prosequences of retroviral proteases including human immunodeficiency virus protease, and sequences recognized and cleaved by trypsin (EP 578472), Takasuga, A. et al. (1992) J. Biochem. 112: 652-57) proteases encoded by Picornaviruses (Ryan, M. D. et al. (1997) J. Gen. Virol. 78: 699-723); factor Xₐ (Gardella, T. J. et al. (1990) J. Biol. Chem. 265: 15854-59; WO 9006370); collagenase (J03280893; WO 9006370; Tajima, S. et al. (1991) J. Ferment. Bioeng. 72: 362); clostripain (EP 578472); subtilisin (including mutant H64A subtilisin, Forsberg, G. et al. (1991) J. Protein Chem. 10: 517-26); chymosin, yeast KEX2 protease (Bourbonnais, Y. et al. (1988) J. Bio. Chem. 263: 15342-47); thrombin (Forsberg et al., supra; Abath, F. G. et al. (1991) BioTechniques 10: 178); Staphylococcus aureus V8 protease or similar endoproteinase-Glu-C to cleave after Glu residues (EP 578472; Ishizaki, J. et al. (1992) Appl. Microbiol. Biotechnol. 36: 483-86); cleavage by NIa proteinase of tobacco etch virus (Parks, T. D. et al. (1994) Anal. Biochem. 216: 413-17); endoproteinase-Lys-C (U.S. Pat. No. 4,414,332); endoproteinase-Asp-N; Neisseria type 2 IgA protease (Pohlner, J. et al. (1992) Biotechnology 10: 799-804); soluble yeast endoproteinase yscF (EP 467839); chymotrypsin (Altman, J. D. et al. (1991) Protein Eng. 4: 593-600); enteropeptidase (WO 9006370), lysostaphin, a polyglycine specific endoproteinase (EP 316748); the family of caspases (i.e. caspase 1, caspase 2, capase 3, etc.); and metalloproteases.

The present invention also contemplates protease recognition sites identified from genomic DNA, cDNA, or random nucleic acid libraries (see for example, O'Boyle, D. R. et al. (1997) Virology 236: 338-47). For example, the fusion nucleic acids of the present invention may comprise a separation site which is a randomizing region for the display of candidate protease recognition sites. The first and second genes of interest encode reporters molecules useful for detecting protease activity, such as GFP molecules capable of undergoing FRET via linkage through a candidate recognition site (see Mitra, R. D. et al. (1996) Gene 173: 13-7). Proteases are expressed or introduced into cells expressing these fusion nucleic acids. Random peptide sequences acting as substrates for the particular protease result in separate GFP proteins which is manifested as loss of FRET signal. By identifying classes of recognition sites, optimal or novel protease recognition sequences may be determined.

In addition to their use in producing separate proteins of interest, the protease cleavage sites and the cognate proteases are also useful in screening for candidate agents that enhance or inhibit protease activity. Since many proteases are crucial to pathogenesis of organisms or cellular regulation, for example HIV or caspase proteases, the ability to express reporter or selection proteins linked by a protease cleavage site allows screens for therapeutic agents directed against a particular protease acting on the recognition site.

Another embodiment of separation sequences is internal ribosome entry sites (IRES). By “internal ribosome entry sites”, “internal ribosome binding sites”, “IRES elements”, or grammatical equivalents as used herein is meant sequences that allow CAP independent initiation of translation (Kim, D. G. et al. (1992) Mol. Cell. Biol. 12: 3636-43; McBratney, S. et al. (1993) Curr. Opin. Cell Biol. 5: 961-65). IRES sequences appear to act by recruiting 40S ribosomal subunit to the mRNA in the absence of translation initiation factors required for normal CAP dependent translation initiation. IRES sequences are heterogenous in nucleotide sequence, RNA structure, and factor requirements for ribosome binding. They are frequently located on the untranslated leader regions of RNA viruses, such as the Picornaviruses. The viral sequences range from about 450-500 nucleotides in length, although IRES sequences may also be shorter or longer (Adam, M. A. et al. (1991) J. Virol. 65: 4985-90; Borman, A. M. et al. (1997) Nucleic Acids Res. 25: 925-32; Hellen, C. U. et al. (1995) Curr. Top. Microbiol. Immunol. 203: 31-63; and Mountford, P. S. et al. (1995) Trends Genet. 11: 179-84). Embodiments of viral IRES separation sites are the Type I IRES sequences present in entero- and rhinoviruses and Type II sequences of cardioviruses and apthoviruses (e.g., encephalomyocarditis virus; see Elroy-Stein, O. et al. (1989) Proc. Natl. Acad. Sci. USA 86: 6126-30; Alexander, L. et al. (1994) Proc. Natl. Acad. Sci. USA 91: 1406-10). Other viral IRES sequences are found in hepatitis A viruses (Brown, K. A. et al. (1994) J. Virol. 68: 1066-74), avian reticuloendotheleliosis virus (Lopez-Lastra, M. et al. (1997) Hum. Gene Ther. 8: 1855-65), Moloney murine leukemia virus (Vagner, S. et al. (1995) J. Biol. Chem. 270: 20376-83), short IRES segments of hepatitis C virus (Urabe, M. et al. (1997) Gene 200: 157-62), and DNA viruses (e.g., Karposi's sarcoma-associated virus, Bieleski, L. et al. (2001) J. Virol. 75: 1864-69).

In addition, preferred embodiments of IRES sequences are non-viral IRES elements found in a variety of organisms including yeast, insects, birds and mammals. Like the viral IRES sequences, cellular IRES sequences are heterogeneous in sequence and secondary structure. Cellular IRES sequences, however, may comprise shorter nucleic acid sequences as compared to viral IRES elements (Oh, S. K. et al. (1992) Genes Dev. 6: 1643-53; Chappell, S. A. et al. (2000) 97: 1536-41). Specific IRES sequences include, but are not limited to, those used for expression of immunoglobulin heavy chain binding protein, transcription factors, protein kinases, protein phosphatases, eIF4G (see Johannes, G. et al. (1999) Proc. Natl. Acad. Sci. USA 96: 13118-23; Johannes, G. et al. (1998) RNA 4: 1500-13), vascular endothelial growth factor (Huez, I. et al. (1989) Mol. Cell. Biol. 18: 6178-90), c-myc (Stoneley, M. et al. (2000) Nucleic Acids Res. 28: 687-94), apoptotic protein Apaf-1 (Coldwell, M. J. et al. (2000) Oncogene 19: 899-905), DAP-5 (Henis-Korenblit, S. et al. (2000) Mol. Cell Bio. 20: 496-506), connexin (Werner, R. (2000) IUBMB Life 50: 173-76), Notch-2 (Lauring, S. A. et al. (2000) Mol. Cell. 6: 939-45), and fibroblast growth factor (Creancier, L. et al. (2000) J. Cell. Biol. 150: 275-81). Since some IRES sequences act or function efficiently in particular cell types, the person skilled in the art will choose IRES elements with relevance to particular cells being used to express the fusion nucleic acid. Moreover, multiple IRES sequences in various combinations, either homomultimeric or heteromultimeric arrangements constructed as tandem repeats or connected via linkers, are useful for increasing efficiency of translation initiation. The combinations of IRES elements comprise at least 2 to 10 or more copies or combinations of IRES sequences, depending on the efficiency of initiation desired.

In addition to their use as separation sequences, IRES elements serve as targets for therapeutic agents since IRES sequences mediate expression of proteins involved in viral pathogenesis or cellular disease states. Thus, the present invention is applicable in screens for candidate agents that inhibit IRES mediated translation initiation events.

Other preferred embodiment of IRES elements are sequences in nucleic acid or random nucleic acid libraries that function as IRES elements. Screens for these IRES type sequences can employ fusion nucleic acids containing bicistronically arranged genes of interest encoding reporter genes, selection genes, or combinations thereof. Genomic DNA, cDNA, or random nucleic acid sequences are inserted between the two reporter or selection genes. After introducing the nucleic acid construct into cells, for example by retroviral delivery, the cells are screened for expression of the downstream gene mediated by functional IRES sequences. Selection is based on expression of the downstream reporter or selection gene (e.g., FACS analysis for expression of a downstream GFP gene). The upstream gene of interest serves to permit monitoring of expression of the fusion nucleic acid. The length of the nucleic acids screened is preferably 6 to 100 nucleotides, although longer nucleic acids may be used.

The present invention further contemplates use of enhancers of IRES mediated translation initiation. IRES initiated translation may be enhanced by any number of methods. Cellular expression of virally encoded proteases, which cleaves eIF4F to remove CAP-binding activity from the 40S ribosome complexes, may be employed to increase preference for IRES translation initiation events. These proteases are found in some Picomaviruses and can be expressed in a cell by introducing the viral protease gene by transfection or retroviral delivery (Roberts, L. O. (1998) RNA 4: 520-29). Other enhancers adaptable for use with IRES elements include cis-acting elements, such as 3′ untranslated region of hepatitis C virus (Ito, T. et al. (1998) J. Virol. 72: 8789-96) and polyA segments (Bergamini, G. et al. (2000) RNA 6: 1781-90), which may be included as part of the fusion nucleic acid of the present invention. In addition, preferential use of cellular IRES sequences may occur when CAP dependent mechanisms are impaired, for example by dephosphorylation of 4E-BP, or when cells are placed under stress by γ-irradiation, amino acid starvation, or hypoxia. Thus, in addition to the methods described above, IRES enhancing procedures include activation or introduction of 4E-BP targeted phosphatases or treating the cells to the stress conditions described above. Other transacting IRES enhancers include heterogeneous nuclear ribonucleoprotein (hnRNP) (Kaminski, A. et al. (1998) RNA 4: 626-38), PTB hnRNP E2/PCBP2 (Walter, B. L. et al. (1999) RNA 5:1570-85), La autoantigen (Meerovitch, K. et al. (1993) J. Virol. 67: 3798-07), unr (Hunt, S. L. et al. (1999) Genes Dev. 13: 437-48), ITAF45/Mpp1 (Pilipenko, E. V. et al. (2000) Genes Dev. 14: 2028-45), DAP5/NAT1/p97 (Henis-Korenblit, S. et al. (2000) Mol. Cell. Biol. 20: 496-506), and nucleolin (Izumi, R. E. et al. (2001) Virus Res. 76: 17-29). These factors may be introduced into a cell either alone or in various combinations. Accordingly, various combinations of IRES elements and enhancing factors are used to effect a separation reaction.

In another preferred embodiment, the separation sites are Type 2A separation sequences. By “Type 2A” sequences herein is meant nucleic acid sequences that when translated inhibit formation of peptide linkages during or following the translation process. Type 2A sequences are distinguished from IRES sequences in that 2A sequences do not involve CAP independent translation initiation. Without being bound by theory, Type 2A sequences appear to act by disrupting peptide bond formation between the nascent polypeptide chain and the incoming activated tRNA^{PRO} (Donnelly, M. L. et al. (2001) J. Gen. Virol 82: 1013-25). Although it is believed that the peptide bond fails to form, the ribosome continues to translate the remainder of the RNA to produce separate peptides unlinked at the carboxy terminus of the 2A peptide region. An advantage of Type 2A separation sequences is that near stoichiometric amounts of first protein of interest and second protein of interest are made as compared to IRES elements. Moreover, Type 2A sequences do not appear to require additional factors, such as proteases, that are required to effect separation when using protease recognition sites.

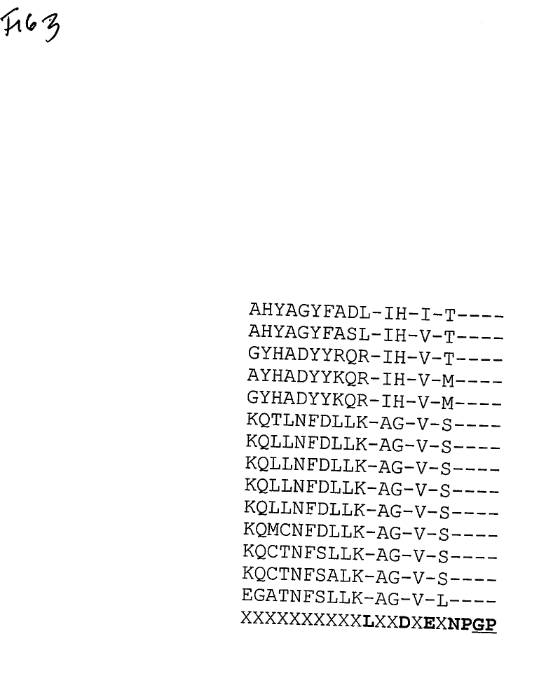

Preferred Type 2A separation sequences are those found in cardioviral and apthoviral genomes, which are approximately 21 amino acids long and have the general sequence XXXXXXXXXXLXXXDXEXNPGP, where X is any amino acid, although amino acids conserved in the family of Type 2A sequences are preferred. Disruption of peptide bond formation occurs between the underlined carboxy terminal glycine (G) and proline (P). These 2A sequences are found in the apthovirus Foot and Mouth Disease Virus (FMDV), cardiovirus Theiler's murine encephalomyelitis virus (TME), and encephalomyocarditis virus (EMC). Various viral Type 2A sequences are shown in FIG. 3. The 2A sequences function in a wide range of eukaryotic expression systems, thus allowing their use in a variety of cells and organisms, such as yeast, worms, insects, plants, and mammals. Accordingly, inserting these 2A separation sequences in between the nucleic acids encoding the first gene of interest and second gene of interest, as more fully explained below, will lead to expression of separate protein products of the first and second gene of interest.

In another embodiment, the present invention contemplates mutated versions or variants of Type 2A sequences. By “mutated” or “variant,” or grammatical equivalents herein is meant deletions, insertions, transitions, transversions of nucleic acid sequences that exhibit the same qualitative separating activity as displayed by the naturally occurring analogue, although preferred mutants or variants have more efficient separating activity and efficient translation of the downstream gene of interest. Mutant variants include changes in nucleic acid sequence that do not change the corresponding 2A amino acid sequence, but incorporate degenerate codons, especially preferred codons of an organism (i.e., codon optimized) for efficient translation of the 2A region (see Zolotukin, S. et al. (1996) J. Virol. 70: 4646-54). In another aspect, the mutant or variants have changes in nucleic acid sequence that change the corresponding 2A amino acid sequence. Thus, preferred embodiments of variant 2A sequences are deletions of the 2A sequence. The deletion may comprise removal of about 3 to 6 amino acids at the amino terminus of the 2A region. In another preferred embodiment, Type 2A sequences are mutated by methods well known in the art, such as chemical mutagenensis, oligonucleotide directed mutagenesis, and error prone replication. Mutants with altered separating activity are readily identified by examining expression of the fusion nucleic acids of the present invention. Assaying for production of a separate downstream gene product, such as a reporter protein or a selection protein, allows for identifying sequences having separating activity. Another method for identifying variants may use a FRET based assay using linked GFP molecules, as described above. Inserting the candidate 2A sequences in or adjacent to the gly-ser linker region, or other suitable regions linking the GFPs, will allow detection of functional 2A separation sequences by identifying constructs that produce separated GFP molecules as measured by loss of FRET signal. Sequences having no or reduced separating activity will retain higher levels of FRET signal due to physical linkage of the GFP molecules. This strategy will permit high throughput analysis of variants and allows selecting for sequences having high efficiency Type 2A separating activity.

In yet another embodiment, Type 2A separation sequences include homologs present in other nucleic acids, including nucleic acids of other viruses, bacteria, yeast, and multicellular organisms such as worms, insects, birds, and mammals. Homology in this context means sequence similarity or identity. A variety of sequence based alignment methodologies, which are well known to those skilled in the art, are useful in identifying homologous sequences. These include, but not limited to, the local homology algorithm of Smith, F. and Waterman, M. S. (1981) Adv. Appl. Math. 2: 482-89, homology alignment algorithm of Peason, W. R. and Lipman, D. J. (1988) Proc. Natl. Acad. Sci. USA 85: 2444-48, Basic Local Alignment Search Tool (BLAST) described by Altschul, S. F. et al. (1990) J. Mol. Biol. 215: 403-10, the Best Fit program described by Devereau, J. et al. (1984) Nucleic Acids. Res. 12: 387-95, and the FastA and TFASTA alignment programs, preferably using default settings or by inspection.

In one preferred embodiment, similarity or identity for any nucleic acid or protein outlined herein is calculated by Fast alignment algorithms based upon the following parameters: mismatch penalty of 1.0; gap size penalty of 0.33; and joining penalty of 30 (see “Current Methods in Comparison and Analysis” in Macromolecule Sequencing and Synthesis: Selected Methods and Applications, p. 127-149, Alan R. Liss, Inc., 1998). Another example of an useful algorithm is PILEUP. PILEUP creates multiple sequence alignment from a group of related sequences using progressive, pairwise alignments. It can also plot a tree showing the clustering relationships used to create the alignment. PILEUP uses a simplification of the progressive alignment method of Feng, D. F. and Doolittle, R. F. (1987) J. Mol. Evol. 25, 351-60, which is similar to the method described by Higgins, D. G. and Sharp, P. M. (1989) CABIOS 5: 151-3. Useful parameters include a default gap weight of 3.00, a default gap length weight of 0.10, and weighted end gaps.

Another example of a useful algorithm is the family of BLAST alignment tools initial described by Altschul et al. (see also Karlin, S. et al. (1993) Proc. Natl. Acad. Sci. USA 90: 5873-87). A particularly useful BLAST program is WU-BLAST-2 program described in Altschul, S. F. et al. (1996) Methods Enzymol. 266: 460-80. WU-BLAST uses several search parameters, most of which are set to default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the longer sequence in the aligned region. The “longer” sequence is one having the most actual residues in the aligned region; gaps introduced by WU-BLAST-2 to maximize the alignment score are ignored.

In a similar manner, “percent (%) nucleic acid sequence identity” with respect to the coding sequence of the polypeptide described herein is defined as the percentage of the nucleotide residues in a candidate sequence that are identical with the nucleotide residues in the coding sequence of the Type 2A regions. A preferred method utilizes the BLASTN module of WU-BLAST-2 set to the default parameters, with overlap span and overlap fraction set to 1 and 0.125, respectively.

An additional useful algorithm is gapped BLAST as reported by Altschul, S. F. et al. (1997) Nucleic Acids Res. 25: 3389-402. Gapped BLAST uses BLOSSOM-62 substitution scores; threshold parameter set to 9; the two-hit method to trigger ungapped extensions; charges gap lengths of k at cost of 10+k; Xu set to 16; and Xg set to 40 for database search stage and to 67 for the output stage of the algorithms. Gapped alignments are triggered by a score corresponding to −22 bits.

The alignment may include the introduction of gaps in the sequence to be aligned. In addition, for sequences which contain either more or fewer amino acids than the Type 2A sequences in FIG. 3, it is understood that the percentage of the homology will be determined based on the number of homologous amino acids in relation to the total number of amino acids. Thus, Type 2A sequences may be shorter or longer than the amino acid sequence shown in FIG. 3.

Another embodiment of Type 2A separating sequences are those sequences present in libraries of nucleic acids, including genomic DNA or cDNA that have Type 2A separating activity. By Type 2A separating activity herein is meant a nucleic acid which encodes a amino acid sequence that exhibits similar separating activity as the naturally occurring Type 2A sequences. Segments of nucleic acids are inserted between the first and second gene of interest in the fusion nucleic acids of the present invention and examined for separating activity as described above. The preferred lengths to be tested are nucleic acids encoding peptides 5 to 50 amino acids or larger, with a more preferred range of peptides 10-30 amino acids long.

Preferred embodiments of Type 2A sequences also encompass random nucleic acids encoding random peptides that have Type 2A separating activity. In these embodiments, the separation site represents a randomizing region where random or biased random nucleic acids encoding random or biased random peptides are inserted between the first gene of interest and second gene of interest. The preferred lengths of the random nucleic acids are nucleic acids encoding peptides 5 to 50 amino acids, with a more preferred range of peptides 10-30 amino acids. Random peptides having separating activity are identified using the above described assays. Identification of functional separating sequences will permit additional searches for related sequences having Type 2A like separating activity, either through homology searches, mutagenesis screens, or by use of biased random peptide sequences. Sequences with separating activity can then be used to express separate proteins of interest according to the present invention.

In a preferred embodiment, the fusion nucleic acids of the present invention comprises genes of interest linked to a fusion partner to form a fusion polypeptide. By “fusion partner” or “functional group” herein is meant a sequence that is associated with the gene of interest, or candidate agent described below, that confers upon all members of the library in that class a common function or ability. Fusion partners can be heterologous (i.e., not native to the host cell), or synthetic (i.e., not native to any cell). Suitable fusion partners include, but are not limited to: (a) presentation structures, as defined below, which provide the peptides of interest and candidate agents in a conformationally restricted or stable form; (b) targeting sequences, defined below, which allow the localization of the genes of interest and candidate agent into a subcellular or extracellular compartment; (c) rescue sequences as defined below, which allow the purification or isolation of either the peptide of interest (for example, when a gene of interest is a peptide) or candidate agents or the nucleic acids encoding them; (d) stability sequences, which confer stability or protection from degradation to the protein of interest or candidate agent or the nucleic acid encoding it, for example resistance to proteolytic degradation; (e) dimerization sequences, to allow for peptide dimerization; or (f) any combination of the above, as well as linker sequences as needed.

In a preferred embodiment, the fusion partner is a presentation structure. By “presentation structure” or grammatical equivalents herein is meant a sequence, when fused to a peptide encoded by gene of interest or peptide candidate agents, causes the peptides to assume a conformationally restricted form. Proteins interact with each other largely through conformationally constrained domains. Although small peptides with freely rotating amino and carboxyl termini can have potent functions as is known in the art, the conversion of such peptide structures into pharmacologically or biologically active agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present short peptide domains or randomized peptide structures.

Presentation structures are preferably used with peptides encoded by genes of interest and peptide candidate agents, although candidate agents, as more fully described below, may be either nucleic acid or peptides. Thus, when presentation structures are used with peptide candidate agents, synthetic presentation structures, i.e., artificial polypeptide, are adaptable for presenting a peptide, for example a randomized peptide, as a conformationally-restricted domain. Generally, such presentation structures comprise a first portion joined to the N-terminal end of the peptide, and a second portion joined to the C-terminal end of the peptide; that is, the peptide is inserted into the presentation structure, although variations may be made, as outlined below. To increase the functional isolation of the peptide expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In a preferred embodiment, the presentation structure is a coiled-coil structure, allowing the presentation of the protein or randomized peptide on an exterior loop (Myszka, D. G. et al. (1994) Biochemistry 33: 2362-73, hereby incorporated by reference). Using this system investigators have isolated peptides capable of high affinity interaction with the appropriate target. In general, coiled-coil structures allow for between 6 to 20 randomized positions.

A preferred coiled-coil presentation structure is as follows: MGCAALESEVSALESVASLESEVAALGRGDMPLAAVKSKLSAVKSKLASVKSKLAACGPP. The underlined regions represent a coiled-coil leucine zipper region defined previously (Martin, F. et al. (1994) EMBO J. 13: 5303-09, hereby incorporated by reference). The bolded GRGDMP region represents the loop structure and may be appropriately replaced with gene of interest (e.g., randomized peptides or peptide interaction domains), generally depicted herein as (X)ₙ, where X is an amino acid residue and n is an integer of at least 5 or 6 and of variable length. The replacement of the bolded region is facilitated by encoding restriction endonuclease sites in the underlined regions, which allows the direct incorporation of genes of interest or randomized oligonucleotides at these positions. For example, a preferred embodiment generates a XhoI site at the double underlined LE site and a HindIII site at the double-underlined KL site.

In a preferred embodiment, the presentation structure is a minibody structure. A “minibody” is essentially composed of a minimal antibody complementarity region. The minibody presentation structure generally provides two sites for insertion of peptides or for randomizing amino acids that in the folded protein are presented along a single face of the tertiary structure (see for example, Bianchi, E. et al. (1994) J. Mol. Biol. 236: 649-59, and references cited therein, all of which are incorporated by reference). Investigators have shown this minimal domain is stable in solution and have used phage selection systems in combinatorial libraries to select minibodies with peptide regions exhibiting high affinity (K_{d}=10⁻⁷) for the pro-inflammatory cytokine IL-6.

A preferred minibody presentation structure is as follows: MGRNSQATSGFTFSHFYMEWVRGGEYIAASRHKHNKYTTEYSASVKGRYIVSRDTSQSILYLQKKKG PP. The bold, underlined regions are the regions which may be randomized. The italicized phenylalanine must be invariant in the first randomizing region. The entire peptide is cloned in a three-oligonucleotide variation of the coiled-coil embodiment, thus allowing two different randomizing regions to be incorporated simultaneously. This embodiment utilizes non-palindromic BstXI sites on the termini.

In a preferred embodiment, the presentation structure is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random peptide sequences, with or without spacer or linking sequences, may be flanked with cysteine residues. In other embodiments, effective presentation structures may be generated by the random regions themselves. For example, the random regions may be “doped” with cysteine residues which, under the appropriate redox conditions, may result in highly cross-linked structured conformations, similar to a presentation structure. Similarly, the randomization regions may be controlled to contain a certain number of residues to confer β-sheet or α-helical structures.

In a preferred embodiment, the presentation sequence confers the ability to bind metal ions to generate a conformationally restricted secondary structure. Thus, for example, C2H2 zinc finger sequences are used; C2H2 sequences have two cysteines and two histidines placed such that a zinc ion is chelated. Zinc finger domains are known to occur independently in multiple zinc-finger peptides to form structurally independent, flexibly linked domains (see Nakaseko, Y. et al. (1992) J. Mol. Biol. 228: 619-36). A general consensus sequence is (5 amino acids)—C—(2 to 3 amino acids)—C—(4 to 12 amino acids)—H—(3 amino acids)—H—(5 amino acids). A preferred example would be —FQCEEC-random peptide of 3 to 20 amino acids-HIRSHTG.

Similarly, CCHC boxes having a consensus sequence —C—(2 amino acids)—C—(4 to 20 random peptide)—H—(4 amino acids)—C— can be used, (see Bavoso, A. et al. (1998) Biochem. Biophys. Res. Commun. 242: 385-89, hereby incorporated by reference). Preferred examples include (1) —VKCFNC-4 to 20 random amino acids-HTARNCR—, based on the nucleocapsid protein P2; (2) a sequence modified from that of the naturally occurring zinc-binding peptide of the Lasp-1 LIM domain (Hammarstrom, A. et al. (1996) Biochemistry 35: 12723-32); and (3) -MNPNCARCG-4 to 20 random amino acids-HKACF—, based on the NMR structural ensemble 1ZFP (Hammarstrom, A et al., supra).

In a preferred embodiment, the fusion partner is a targeting sequence. As will be appreciated by those in the art, the localization of proteins within a cell is a simple method for increasing effective concentration and determining function. For example, RAF-1 targeted to the mitochondrial membrane can inhibit the anti-apoptotic effect of BCL-2. Similarly, membrane bound Sos induces Ras mediated signaling in T-lymphocytes. These mechanisms are thought to rely on the principle of limiting the search space for ligands. In other words, the localization of a protein to the plasma membrane limits the search for its ligand to that restricted dimensional space near the membrane as opposed to the three dimensional space of the cytoplasm. Alternatively, the concentration of a protein can also be simply increased by nature of the localization. Shuttling the proteins into the nucleus confines them to a smaller volume thereby increasing concentration. Finally, the ligand or target may simply be localized to a specific compartment, and cognate inhibitors localized appropriately.

Thus, suitable targeting sequences include, but are not limited to, affinity sequences capable of causing binding of the expression product to a predetermined molecule or class of molecules while retaining bioactivity of the expression product (e.g., by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including (a) subcellular locations such as the Golgi, endoplasmic reticulum, nucleus, nucleoli, nuclear membrane, mitochondria, chloroplast, secretory vesicles, lysosome, and cellular membrane, and (b) extracellular locations via a secretory signal. Particularly preferred is localization to either subcellular locations or to the outside of the cell via secretion.

In a preferred embodiment, the targeting sequence comprises a nuclear localization signal (NLS). NLSs are generally short, positively charged (basic) domains that serve to direct the entire protein in which they occur to the cell's nucleus. Numerous NLS amino acid sequences have been reported including single basic NLS's such as that of the SV40 (monkey virus) large T Antigen (PKKKRKV, Kalderon, D. et al. (1984) Cell 39: 499-509); the human retinoic acid receptor-β nuclear localization signal (ARRRRP), NFkB p50 (EEVQRKRQKL, Ghosh, S. et al. (1990) Cell 62: 1019-29); NFkB p65 (EEKRKRTYE, Nolan, G. et al. (1991) Cell 64: 961-99; and others (see for example Boulikas, T. (1994) J. Cell. Biochem. 55: 32-58, hereby incorporated by reference) and double basic NLS's exemplified by that of the Xenopus (African clawed toad) protein, nucleoplasmin (AVKRPAATKKAGQAKKKKLD, Dingwall, C. et al. (1982) Cell, 30: 449-58, and Dingwall, S. et al. (1988) J. Cell Biol. 107: 641-49). Numerous localization studies have demonstrated that NLSs incorporated in synthetic peptides or grafted onto proteins not normally targeted to the cell nucleus cause these peptides and proteins to concentrate in the nucleus (see Dingwall S. et al. (1986) Ann. Rev. Cell Biol. 2: 367-90; Bonnerot, C. et al. (1987) Proc. Natl. Acad. Sci. USA 84: 6795-99; Galileo, D. S. et al. (1990) Proc. Natl. Acad. Sci. USA 87: 458-62.)

In a preferred embodiment, the targeting sequence comprises a membrane anchoring signal sequence. These sequences are particularly useful since many intracellular events originate at the plasma membrane and many parasites and pathogens bind to the membrane during pathogenesis. Thus, membrane-bound peptide libraries are useful for both for the identification of important elements in these processes as well as for the discovery of effective inhibitors. The invention provides methods for presenting extracellularly or in the cytoplasmic space the randomized peptide candidate agent or a peptide encoded by a gene of interest. For extracellular presentation, a membrane anchoring region is provided at the carboxyl terminus of the peptide presentation structure. The peptide or randomized expression product region is expressed on the cell surface and presented to the extracellular space, such that it can bind to other surface molecules affecting their function or molecules present in the extracellular medium. The binding of such molecules could confer function on the cells expressing a peptide that binds the molecule. The cytoplasmic region could be neutral or could contain a domain that, when the extracellular expression product region is bound, confers a function on the cells (e.g., activation of a kinase, phosphatase, binding of other cellular components to effect function). Similarly, a region containing the peptide of interest or randomized peptide could be confined within the cytoplasmic compartment, and the transmembrane region and extracellular region remain constant or have a specified function.

Membrane-anchoring sequences are well known in the art and are based on the genetic geometry of mammalian transmembrane molecules. Peptides are inserted into the membrane via a signal sequence (designated herein as ssTM) and stably held in the membrane through a hydrophobic transmembrane domain (TM). The transmembrane proteins are positioned in the membrane such that the protein region encompassing the amino terminus relative to the transmembrane domain are extracellular and the region towards the carboxy terminal are intracellular. Of course, if the position of transmembrane domains is towards the amino end of the protein relative to the variable region, the TM will serve to position the variable region or protein of interest intracellularly, which may be desirable in some embodiments. ssTMs and TMs are known for a wide variety of membrane bound proteins, and these sequences are used accordingly, either as pairs from a particular protein or with each component being taken from a different protein. Alternatively, the ssTM and TM sequences are synthetic and derived entirely from consensus sequences, thus serving as artificial delivery domains.

As will be appreciated by those in the art, membrane-anchoring sequences, including both ssTM and TM, are known for a wide variety of proteins and any of these are useful in the present invention. Particularly preferred membrane-anchoring sequences include, but are not limited to, those derived from CD8, ICAM-2, IL-8R, CD4, and LFA-1. Other useful ssTM and TM domains include sequences from: (a) class I integral membrane proteins such as IL-2 receptor beta-chain (residues 1-26 are the signal sequence, 241-265 are the transmembrane residues; see Hatakeyama, M. et al. (1989) Science 244: 551-56 and von Heijne, G. et al. (1988) Eur. J. Biochem. 174: 671-78) and insulin receptor beta chain (residues 1-27 are the signal domain, 957-959 are the transmembrane domain and 960-1382 are the cytoplasmic domain; see Hatakeyama, supra, and Ebina, Y. et al. (1985) Cell 40: 747-58); (b) class II integral membrane proteins such as neutral endopeptidase (residues 29-51 are the transmembrane domain, 2-28 are the cytoplasmic domain; see Malfroy, B. et al. (1987) Biochem. Biophys. Res. Commun. 144: 59-66); (c) type III proteins such as human cytochrome P450 NF25 (Hatakeyama, supra); and (d) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP, Nakauchi, H. et al. (1985) Proc. Natl. Acad. Sci. USA 82: 5126-30) and amino acid 1-21 in the case of ICAM-2 (MSSFGYRTLTVALFTLICCPG, Staunton, D. E. et al. (1989) Nature 339: 61-64). These leader sequences deliver the construct to the membrane while the hydrophobic transmembrane domains placed at the carboxy terminal region relative to the peptide of interest or peptide candidate agents serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR, Nakauchi, et al., supra) and 224-256 from ICAM-2 (MVIIVTVVSVLLSLFVTSVLLCFIFGQHLRQQR, Staunton, et al., supra).

Alternatively, membrane anchoring sequences include the GPI anchor, a covalently bound glycosyl-phosphatidylinositol moiety localizing the modified protein to the lipid bilayer. The GPI anchor sequence is exemplified by protein DAF, which comprises the sequence PNKGSGTTSGTTRLLSGHTCFTLTGLLGTLVTMGLLT, with the bolded serine the site of the anchor; (see Homans, S. W. et al. (1988) Nature 333: 269-72, and Moran, P. et al. (1991) J. Biol. Chem. 266: 1250-57). GPI modification is accomplished by inserting a GPI anchor sequence from a variety of GPI modified proteins, including those of Thy-1, TAG1, N-CAM, F11, and other alike onto the carboxy terminal region relative to the inserted peptide of interest or inserted random peptide. Thus, the GPI anchor sequences replaces the transmembrane domain in these constructs. The GPI anchor sequences may also comprise synthetic sequences that serve as GPI modification sites (see Coyne, K. E. et al. (1993) J. Biol. Chem. 268: 6689-93).

Similarly, acylation signals for attachment of lipid moieties can also serve as membrane anchoring sequences (see Stickney, J. T. (2001) Methods Enzymol. 332: 64-77). It is known that the myristylation of c-src localizes the kinase to the plasma membrane. This property provides a simple and effective method of membrane localization given that the first 14 amino acids of the protein are solely responsible for this function: MGSSKSKPKDPSQR (see Cross, F. R. et al. (1984) Mol. Cell. Biol. 4: 1834-42 and Spencer, D. M. et al. (1993) Science 262: 1019-24, both of which are hereby incorporated by reference) or MGQSLTTPLSL. The modification at the glycine residue (in bold) of the motif is effective in localizing reporter genes and can be used to anchor the zeta chain of the TCR. The myristylation signal motif is placed at the amino end relative to the variable region or protein of interest in order to localize the construct to the plasma membrane. Another lipid modification is isoprenoid attachment, which includes the 15 carbon farnesyl or the 20 carbon geranyl-geranly group. The conserved sequence for isoprenoid attachment comprises CaaX motif with the cysteine residue as the lipid modified amino acid. The X residue determines the type of isoprenoid modification. The preferred isoprenoid is geranyl-geranyl when X is a leucine or phenylalanine (Farnsworth, C. C. et al. (1994) Proc. Natl. Acad. Sci. USA 91: 11963-67). Farnesyl is the preferred lipid for a broader range of X amino acids such as methionine, serine, glutamine, and alanine. The “aa” in the isoprenoid attachment motif are generally aliphatic residues, although other residues are also functional. Farnesylation sequences include carboxy terminal SKDGKKKKKKSKTKCVIM of K-Ras4B. Other isoprenoid attachment motifs are found in the carboxy termini of N and H-Ras GTPases.

In addition, localization to the cell membrane by lipid modification is also achieved by palmitoylation. Attachment of the palmitoyl group can be directed to either the amino or carboxy terminal region relative to the protein of interest. In addition, multiple palmitoyl residues or combinations of palmitoyl and isoprenoid attachments are possible. Amino terminal additions of palmitoyl group may use the sequence MVCCMRRTKQV from Gap43 protein while carboxy terminal modifications are possible with CMSCKCVLKKKKKK from Ras mutant (modified amino acids in bold). Other palmitoylation sequences are found in G protein-coupled receptor kinase GRK6 sequence (LLQRLFSRQDCCGNCSDSEEELPTRL, Stoffel, R. H. et al. (1994) J. Biol. Chem. 269: 27791-94); rhodopsin (KQFRNCMLTSLCCGKNPLGD, Barnstable, C. J. et al. (1994) J. Mol. Neurosci. 5: 207-09); and the p21 H-ras 1 protein (LNPPDESGPGCMSCKCVLS, Capon, D. J. et al. (1983) Nature 302: 33-37). Use of the carboxy terminal sequence LNPPDESGPGC(p)MSC(p)KC(f)VLS of H-Ras (modified amino acids in bold; p is palmitoyl group and f is farnesyl group) allows attachment of both palmitoyl and farnesyl lipids

In a preferred embodiment, the targeting sequence comprises a lysosomal targeting sequence, including, for example, a lysosomal degradation sequence such as Lamp-2 (KFERQ, Dice, J. F. (1992) Ann. N.Y. Acad. Sci. 674: 58-64); lysosomal membrane sequences from Lamp-1 (MLIPIAGFFALAGLVLIVLIAYLIGRKRSHAGYQTI, Uthayakumar, S. et al. (1995) Cell. Mol. Biol. Res. 41: 405-20); or h-Lamp-2 (LVPIAVGAALAGVLILVLLAYFIGLKHHHAGYEQF, Konecki, D. S. et al. (1994) Biochem. Biophys. Res. Comm. 205: 1-5; where italicized residues comprise the transmembrane domains and underlined residues comprise the cytoplasmic targeting signal).

Alternatively, the targeting sequence may be a mitochondrial localization sequence, including mitochondrial matrix sequences (yeast alcohol dehydrogenase III; MLRTSSLFTRRVQPSLFSRNILRLQST, Schatz, G. (1987) Eur. J. Biochem. 165:1-6); mitochondrial inner membrane sequences (yeast cytochrome c oxidase subunit IV; MLSLRQSIRFFKPATRTLCSSRYLL, Schatz, supra); mitochondrial intermembrane space sequences (yeast cytochrome c1; MFSMLSKRWAQRTLSKSFYSTATGAASKSGKLTQKLVTAGVAAAGITASTLLYADSLTAEAMTA, Schatz, supra); or mitochondrial outer membrane sequences (yeast 70 kD outer membrane protein; MKSFITRNKTAILATVAATGTAIGAYYYYNQLQQQQQRGKK, Schatz, supra).

The targeting sequences may also comprise endoplasmic reticulum sequences, including the sequences from calreticulin (KDEL, Pelham, H. R. (1992) Royal Society London Transactions B; 1-10) and adenovirus E3/19K protein (LYLSRRSFIDEKKMP, Jackson, M. R. et al. (1990) EMBO J. 9: 3153-62). Furthermore, targeting sequences also include peroxisome sequences (for example, the peroxisome matrix sequence of luciferase, SKL (Keller, G. A. et al. (1987) Proc. Natl. Acad. Sci. USA 4: 3264-68).

In a preferred embodiment, the targeting sequence comprises a secretory signal sequence capable of effecting the secretion of the peptide of interest or peptide candidate agent. There are a large number of known secretory signal sequences capable of directing secretion of the peptide into the extracellular space when placed at the amino end relative to the peptide of interest. Secretory signal sequences and their transferability to unrelated proteins are well known (see Silhavy, T. J. et al. (1985) Microbiol. Rev. 49: 398-418). Secretion of the peptide is particularly useful to generate peptides capable of binding to the surface or affecting the physiology of a target cell other than the host cell, i.e., the cell infected with the retrovirus. In a preferred approach, a fusion product is configured to contain, in series, secretion signal peptide-presentation structure-randomized peptide region or protein of interest-presentation structure. In this manner, target cells grown in the vicinity of cells expressing the library of peptides are exposed to the secreted peptide. Target cells exhibiting a physiological change in response to the presence of the secreted peptide (e.g., by the peptide binding to a surface receptor or by being internalized and binding to intracellular targets) and the peptide secreting cells are localized by any of a variety of selection schemes and the structure of the peptide effector identified. Exemplary effects include that of a designer cytokine (e.g., a stem cell factor capable of causing hematopoietic stem cells to divide and maintain their totipotential), a factor causing cancer cells to undergo spontaneous apoptosis, a factor that binds to the cell surface of target cells and labels them specifically, etc.

Suitable secretory sequences are known, including signals from IL-2 (MYRMQLLSCIALSLALVTNS, Villinger, F. et al. (1995) J. Immunol. 155: 3946-54), growth hormone (MATGSRTSLLLAFGLLCLPWLQEGSAFPT, Roskam, W. G. et al. (1979) Nucleic Acids Res. 7: 305-20); preproinsulin (MALWMRLLPLLALLALWGPDPAAAFVN, Bell, G. I. et al. (1980) Nature 284: 26-32); and influenza HA protein (MKAKLLVLLYAFVAGDQI, Sekiwawa, K. et al. (1983) Proc. Natl. Acad. Sci. USA 80: 3563-67), with cleavage between the non-underlined-underlined junction. A particularly preferred secretory signal sequence is the signal leader sequence from the secreted cytokine IL-4, MGLTSQLLPPLFFLLACAGNFVHG, which comprises the first 24 amino acids of IL-4.

In a preferred embodiment, the fusion partner comprises a rescue sequence. A rescue sequence is a sequence which may be used to purify or isolate either the peptide of interest or the candidate agent or the nucleic acid encoding it. Thus, for example, peptide rescue sequences include purification sequences such as the His₆ tag for use with Ni⁺² affinity columns and epitope tags useful for detection, immunoprecipitation, or FACS (fluorescence-activated cell sorting). Suitable epitope tags include myc (for use with the commercially available 9E10 antibody), the BSP biotinylation target sequence of the bacterial enzyme BirA, flu tags, lacZ, GST, and Strep tag I and II.

Alternatively, the rescue sequence may be a unique oligonucleotide sequence which serves as a probe target site to allow the facile isolation of the retroviral construct, via PCR, related techniques, or by hybridization.

In a preferred embodiment, the fusion partner comprises a stability sequence which affects the stability to the peptide of interest or candidate bioactive agent. In one aspect, the stability sequence confers stability to the peptide of interest or candidate bioactive agent. For example, peptides may be stabilized by the incorporation of glycines after the initiating methionine (MG or MGG), for protection of the peptide from ubiquitination as per Varshavsky's N-End Rule, thus conferring increased half-life in the cell (see Varshavsky, A. (1996) Proc. Natl. Acad. Sci. USA 93: 12142-49). Similarly, adding two prolines at the C-terminus makes peptides resistant to carboxypeptidase action. The presence of two glycines prior to the prolines impart both flexibility and prevent structure perturbing events in the di-proline from propagating into the peptide structure. Thus, preferred stability sequences are MG(X)ₙGGPP, where X is any amino acid and n is an integer of at least four.

In another aspect, the stability sequence decreases the stability of the peptide of interest or candidate bioactive agent. Sequences, such as PEST sequences (i.e., polypeptide sequences enriched in proline (P), glutamic acid (E), serine (S) and threonine (T); see Rechsteiner, M. (1996) Trends Biochem. Sci. 21: 267-71) and destruction boxes (Glotzer, M. (1991) Nature 349 132-38) destabilize proteins by targeting proteins for degradation. For example, fusion of PEST sequences to GFP reporter protein decreases the half-life of GFP, thus providing an indicator of dynamic cellular processes, including, but not limited to, regulated protein degradation, gene transcriptional activity, and cell cycle status (Mateus, C. et al. (2000) Yeast 16: 1313-23; Li. X. (1998) J. Biol. Chem. 273: 34970-75). Numerous PEST sequences useful for targeting peptides for degradation are known. These include amino acids 422-461 of ornithine decarboxylase (Corish, P. (1999) Protein Eng. 12: 1035-40) and the C terminal sequences of IκBα (Lin, R. (1996) Mol. Cell Biol. 16: 1401-09). Destruction boxes found in cell cycle related proteins, for example cyclin B1, can also reduce the half-life of fusion proteins but in a cell cycle dependent manner (RTALGDIGN, Klotzbucher, A. et al. (1996) EMBO J. 1: 3053-64; Corish, P., supra).

In a further preferred embodiment, the stability sequences affect stability of the expressed nucleic acids. A variety of factors are known to affect the stability of RNAs, including 5′ untranslated leader regions (see for example, Poon, M. et al. (1999) Mol Cell Biol 19: 6471-8) and 3′ untranslated terminal sequences (see for example, Chen, C. Y. et al. (1995) Mol Cell Biol 15: 5777-88; Zhou, Q. et al. (1998) Mol Cell Biol 18: 815-26). Stability sequences also include 5′ CAP sites (Konarska, M. M. et al. (1984) Cell 38: 731-6), 3′ polyadenylation signal sequences, and intron sequences (see Wilusz, C. J. (2001) Nat. Rev. Mol. Cell. Biol. 2: 237-46). These sequences may be incorporated into the fusion nucleic acids to destabilize or stabilize the expressed nucleic acids accordingly.

In another embodiment, the fusion partner is a multimerization sequence. A multimerization sequence allows non-covalent association of one peptide of interest to another peptide of interest, with sufficient affinity to remain associated under normal physiological conditions. This effectively allows small libraries of peptides encoded by genes of interest or peptide candidate agents (for example, 10⁴) to become large libraries if, for example, two peptides per cell are generated which then dimerize, to form an effective library of 10⁸ (10⁴×10⁴). It also allows the formation of longer random peptides, if needed, or more structurally complex random peptide molecules. The multimers may be homo- or heteromeric. One preferred multimerization sequences are dimerization sequences.

Multimerization or dimerization sequences may be a single sequence that self-aggregates, or two sequences, each of which is present in the fusion nucleic acid comprising first gene of interest and second gene of interest. Alternatively, the multimerization sequences are present in different retroviral constructs, with each construct expressing a different gene of interest with multimerization sequences. Thus, in various embodiments, nucleic acids encode a first peptide with dimerization sequence 1, and a second peptide with dimerization sequence 2, such that upon introduction into a cell and expression of the nucleic acids, dimerization sequence 1 associates with dimerization sequence 2 to form a new peptide structure or peptide candidate agent. Alternatively, two or more different multimerization sequences may be incorporated into individual gene of interest or candidate peptide agent. For example, a first multimerization sequence may be placed at the amino terminus while a second multimerization sequence is placed at the carboxy terminus. Expression of the protein or peptide allows formation of a variety of complex multiprotein associations, including protein concatemers. Moreover, the use of dimerization sequences allows the noncovalent “constraint” of the random peptides; that is, if a dimerization sequence is used at each terminus of the peptide, the resulting structure can form a constrained structure. For example, the use of dimerizing sequences fused to both the N- and C-terminus of the scaffold such as rGFP or pGFP forms a noncovalently constrained scaffold random peptide library.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods. Particularly preferred dimerization peptide sequences include, but are not limited to, -EFLIVKS—, EEFLIVKKS—, —FESIKLV—, and —VSIKFEL. More preferred dimerization peptide sequences include EEEFLIVEEE when used together with KKKFLIVKKK.

The fusion partners may be placed anywhere (i.e., N-terminal, C-terminal, internal) in the structure as the biology and activity permits.

In a preferred embodiment, the fusion partner includes a linker or spacer sequence. Linker sequences between various targeting sequences (e.g. membrane targeting sequences) and the other components of the constructs, such as the randomized peptides, may be desirable to allow unhindered interaction between peptides and potential targets. For example, useful linkers include glycine polymers (G)ₙ, glycine-serine polymers (including, for example, (GS)ₙ, (GSGGS)ₙ, and (GGGS)ₙ, where n is an integer of at least one), glycine-alanine polymers, alanine-serine polymers, and other flexible linkers such as the tether for the Shaker K⁺ channel, and a large variety of other flexible linkers, as will be appreciated by those in the art. Glycine and glycine-serine polymers are preferred since both of these amino acids are relatively unstructured, and therefore may be able to serve as a neutral tether between components. Glycine polymers are the most preferred as glycine accesses significantly more phi-psi space than even alanine, and is much less restricted than residues with longer side chains (see Scheraga, H. A. (1992) Rev. Computational Chem. III 73-142). Secondly, serine is hydrophilic and therefore able to solubilize what could be a globular glycine chain. Third, similar chains are known to be effective in joining subunits of recombinant proteins such as single chain antibodies.

In addition, the fusion partners, including presentation structures, may be modified, randomized, and/or mutated to alter the presented or displayed orientation of the randomized expression product. For example, determinants at the base of the loop may be modified to slightly modify the internal loop peptide tertiary structure in order to properly display the a peptide, such as a randomized amino acid sequence.

In a preferred embodiment, combinations of fusion partners are used. Thus, for example, any number of combinations of presentation structures, targeting sequences, rescue sequences, and stability sequences may be used, with or without linker sequences. By using a base vector that contains cloning sites for receiving libraries of genes of interest or candidate agents, one can cassette in various fusion partners 5′ and 3′ of a protein or peptide, including a libraries of random peptides. As will be appreciated by those in the art, these modules of sequences can be used in a large number of combinations and variations. In addition, as discussed herein, it is possible to have more than one variable region in a construct, either together to form a new surface or to bring two other molecules together. Alternatively, no presentation structure is used, giving a “free” or “non-constrained” peptide or expression product.

Accordingly, in one preferred embodiment of the present invention, the first gene of interest is a nucleic acid which encodes fusion protein comprising a first fusion partner and a first reporter gene and the second gene of interest comprises a second fusion protein comprising a second fusion partner and second reporter gene. If the fusion partners comprise different cellular localization sequences, such as nuclear localization and membrane localization sequences, the presence of a separation sequence between the first gene of interest and the second gene of interest results in synthesis of separate proteins products capable of localizing to different cellular structures. For example, the described construct allows detecting cells by the nuclearly localized first fusion protein while permitting analysis of cellular morphology or cellular processes by the membrane localized second reporter gene. In complex cell cultures, such as hippocampal slices used for examine learning and memory and synaptic plasticity, tracing the neuronal projections of specific neuronal cells types is particularly important. The described construct allows identifying particular cells by the nuclearly localized first reporter gene and tracing of neuronal projections by the second reporter gene. Those skilled in the art will appreciate that use of different combinations of fusion partners and genes of interest permits monitoring of multiple cellular processes simultaneously. Similarly, targeting of proteins of interest to distinct cellular locations, either intracellulary or extracellularly, is useful in directing proteins to regions where they will be biologically active.

As will be appreciated by those skilled in the art, the retroviral vectors comprising fusion nucleic acids are not limited to fusion nucleic acids comprising only promoter, first gene of interest, separation sequence, and second gene of interest. Any number of separation sequences and genes of interest may be used in the fusion nucleic acids. Additional separating sequences may be chosen from protease based, IRES based, or Type 2A based separating sequences and added to the fusion nucleic acids along with additional genes of interest. Consequently, a preferred embodiment further comprises a second separating sequence and a third gene of interest, and may further comprise a third separating sequence and a fourth gene of interest. As can be appreciated by those skilled in the art, by inserting additional separating sequences and additional genes of interest to the nucleic acids of the present invention, any number of proteins may be separately expressed from the fusion nucleic acid. The additional genes of interest may be identical or non-identical to the first and second genes of interest. These constructs may be desired in screening methods where the first and second gene of interest encode reporter proteins whose activity is affected by an expressed third gene of interest or where expression of more than two genes of interest is necessary to produce a cellular phenotype.

As the objects of the present invention are retroviral vectors that express fusion nucleic acids capable of producing a plurality of protein products not linked by a peptide bond, the present invention further provides for libraries of retroviral vectors comprising fusion nucleic acids comprising a first gene of interest, a separation sequence, and a second gene of interest. Additional embodiments of libraries of retroviral vectors may contain fusion nucleic acids comprising additional separation sequences and genes of interest, as outlined above.

In one embodiment, the libraries of retroviral vectors comprise genes of interest comprising genomic nucleic acids. As described above, genomic nucleic acid libraries are obtainable from any number of different cells, particularly those outlined for host cells of retroviral vectors. The genomic libraries may be generated from eucaryotic and procaryotic cells, viruses, cells infected with viruses or other pathogens, genetically altered cells, etc. Preferred embodiments, as outlined below, include genomic libraries made from different individuals, such as different patients, particularly human patients. The genomic libraries may be complete libraries or partial libraries. Furthermore, a library of candidate agents can be derived from a single genomic source or multiple sources; that is, genomic DNA from multiple cell types or multiple individuals or multiple pathogens can be combined in a screen. The genomic library may utilize entire genomic constructs or fractionated constructs (e.g., genomic DNA fragments), including random or targeted fractionation. Suitable fractionation techniques include enzymatic, chemical or mechanical fractionation.

In another preferred embodiment, the libraries of retroviral vectors comprise genes of interest comprising cDNAs. The cDNA libraries can be derived from any number of different cells and include cDNA libraries generated from eucaryotic and procaryotic cells, viruses, cells infected with viruses or other pathogens, genetically altered cells, etc. Preferred embodiments, as outlined below, include cDNA libraries made from different individuals, such as different patients, particularly human patients.

The cDNA libraries may be complete libraries or partial libraries, including cDNA fragments. Furthermore, the library of candidate proteins can be derived from a single cDNA source or multiple sources; that is, cDNA from multiple cell types or multiple individuals or multiple pathogens can be combined in a screen. The cDNA library may utilize entire cDNA constructs or fractionated constructs, including random or targeted fractionation. Suitable fractionation techniques include enzymatic (i.e. DNase I), chemical, or mechanical fractionation (i.e. sonicated or sheared). Also useful for the present invention are cDNA libraries enriched for a specific class of proteins, such as type I membrane proteins (Tashiro, K. et al. (1993) Science 261: 600-03) and membrane proteins (Kopczynski C. C. (1998) Proc. Natl. Acad. Sci. USA 95: 9973-78). Additionally, fractionation techniques include subtracted cDNA libraries in which genes preferentially or exclusively expressed in particular cells, tissues, or developmental phases are enriched. Methods for making subtracted cDNA libraries are well known in the art (see Diatchenko, L. et al. (1999) Methods Enzymol. 303: 349-80; von Stein, O. D. et al. (1997) Nucleic Acids Res. 13: 2598-602: Carcinci, P. (2000) Genome Res. 10: 1431-32). Generally, in the case of genomic or cDNA libraries, the nucleic acid may have the potential to encode a protein ranging from twenty amino acids to thousands, with from about 50-1000 being preferred and from about 100-500 being especially preferred.

In addition, the genes of interest or candidate agents comprising cDNA or genomic DNA may also be subsequently mutated using known techniques; for example, by exposure to mutagens, error prone PCR, error prone transcription, combinatorial splicing (e.g., cre-lox recombination) to generate novel protein sequences. In this way libraries of procaryotic and eukaryotic proteins may be made for screening in the systems described herein. Particularly preferred in this embodiment are libraries of bacterial, fungal, viral and mammalian proteins, with the latter being preferred, and human proteins being especially preferred.

In another preferred embodiment, the libraries of retroviral vectors comprise genes of interest comprising nucleic acids encoding random or biased random peptide library. Generally, encoded peptides ranging from about 4 amino acids in length to about 100 amino acids may be used, with peptide ranging from about 5 to 50 being preferred, with from about 5 to 30 being particularly preferred and from about 6 to about 15 especially being preferred. Since random or biased random peptide libraries are also sources of candidates agents described below, the following discussion of random or biased random peptides apply equally well to candidate agents.

The nucleic acids encoding the peptides are randomized, either fully randomized or they are biased in their randomization, i.e., in nucleotide/residue frequency generally or per position. By “randomized” or grammatical equivalents herein is meant that each nucleic acid and peptide consists of essentially random nucleotides and amino acids, respectively. As is more fully described below, the nucleic acids giving rise to the expression products are chemically synthesized, and thus may incorporate any nucleotide at any position. Thus, when the nucleic acids comprising the genes of interest are expressed to produce peptides, any amino acid residue may be incorporated at any position. The synthetic process can be designed to generate randomized nucleic acids, to allow the formation of all or most of the possible combinations over the length of the nucleic acids, thus forming a library of randomized nucleic acids or peptides.

The library should provide a sufficiently structurally diverse population of randomized expression products to effect a probabilistically sufficient range to provide one or more peptide products which has the desired properties, such as binding to protein interaction domains or producing a desired cellular response. Accordingly, a library must be large enough so that at least one of its members will have a structure that gives it affinity for some molecule, protein, or other factor whose activity is involved in some cellular response, such as signal transduction. Although it is difficult to gauge the required absolute size of an interaction library, nature provides a hint with the immune response: a diversity of 10⁷-10⁸ different antibodies provides at least one combination with sufficient affinity to interact with most potential antigens encountered by an organism. Published in vitro selection techniques have also shown that a library size of about 10⁷ to 10⁸ is sufficient to find structures with affinity for the target. A library of all combinations of a peptide 7-20 amino acids in length, such as proposed here for expression in retroviruses, has the potential to code for 20⁷ (10⁹) to 20²⁰. Thus, with libraries of 10⁷ to 10⁸ per ml of retroviral particles, the present methods is capable of producing a “working” subset of a theoretically complete interaction library for 7 amino acids, and a subset of shapes for the 20²⁰ library. Thus in a preferred embodiment, at least 10⁶, preferably at least 10⁷, more preferably at least 10⁸ and most preferably at least 10⁹ different expression products are simultaneously analyzed in the subject methods. Preferred methods maximize library size and diversity.

It is important to understand that in any library system encoded by oligonucleotide synthesis one cannot have complete control over the codons that will eventually be incorporated into the peptide structure. This is especially true in the case of codons encoding stop signals (TAA, TGA, TAG). In a synthesis with NNN as the random region, there is a 3/64, or 4.69% chance that the codon will be a stop codon. Thus, in a peptide of 10 residues, there is an unacceptable high degree of probability that 46.7% of the peptides will prematurely terminate. For free peptide structures this is perhaps not a problem. But for larger structures, such as those envisioned here, such termination will lead to sterile peptide expression. To alleviate this problem, random residues are encoded as NNK, where K=T or G. This allows for encoding of all potential amino acids, changing their relative representation slightly, but importantly preventing the encoding of two stop residues TAA and TGA. Thus, libraries encoding a 10 amino acid peptide will have a 15.6% chance of terminating prematurely. For candidate nucleic acids that are not designed to result in peptide expression products, as described below, this is not necessary.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross linking, prolines for SH3 domains, serines, threonines, tyrosines, or histidines for phosphorylation sites, etc.

In a preferred embodiment, the bias is toward peptides or nucleic acids that interact with known classes of molecules. For example, when the gene of interest or candidate bioactive agent is a peptide, it is known that much of intracellular signaling is carried out by short regions of a polypeptide interacting with other polypeptide regions of other proteins, such as the interaction domains described above. Another example of an interaction domain is a short region from the HIV-1 envelope cytoplasmic domain that has been previously shown to block the action of cellular calmodulin. Regions of the Fas cytoplasmic domain, which shows homology to the mastoparn toxin from Wasps, can be limited to a short peptide region with death inducing apoptotic or G protein inducing functions. Magainin, a natural peptide derived from Xenopus, can have potent anti-tumor and anti-microbial activity. Short peptide fragments of a protein kinase C isozyme (β-PKC) have been shown to block nuclear translocation of PKC in Xenopus oocytes following stimulation. In addition, short SH-3 target proteins have been used as pseudosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate bioactive agents as well.

Thus, a number of molecules or protein domains that confer common function, structure or affinity are suitable as a starting point for generating biased genes of interest or candidate agents. In addition to protein-protein interaction domains, there are a number of nucleic acid interaction domains suitable for use as starting points for biased random peptides. For example, these include leucine zipper domain, homeo box domain, zinc finger domain, and paired domain. As is appreciated by those in the art, while variations of any interaction domains may have weak amino acid homology, the variants may have strong structural homology.

As the present invention comprises libraries of retroviral vectors, the present invention further provides for cells and cellular libraries of retroviral vectors comprising the fusion nucleic acids outlined above. The cells and cellular libraries are generated by introducing the retroviral vectors into a plurality of cells. By a “plurality” of cells herein is meant at least two cells, with at least about 10³ being preferred, at least about 10⁶ being particularly preferred, and at least about 10⁸ to 10⁹ being especially preferred. This plurality of cells comprises a cellular library, wherein generally each cell within the library contains a member of the retroviral molecular library (i.e., different random peptides, cDNA fragments, reporter genes, and other genes of interest, and combinations thereof). As will be appreciated by those in the art, some cells within the library may not contain a retrovirus, and some may contain more than one When methods other than retrovrial infection are used to introduce the fusion nucleic acids into a plurality of cells, the distribution of candidate nucleic acids within the individual members of the cellular library may vary widely, as it is generally difficult to control the number of nucleic acids which enter a cell by other methods such as electroporation or transfection.

The fusion nucleic acids of the present invention and any retroviral constructs described herein can be prepared using standard recombinant DNA techniques described in, for example, Sambrook, J. et al., Molecular Cloning; A Laboratory Manual, 2nd edition, Cold Spring Harbor Press, Cold Spring Harbor, N.Y.,1989, and Ausubul, F. et al., Current Protocols in Molecular Biology, Greene Publishing Associates and John Wiley & Sons, New York, N. Y.,1994. Generally, the vectors also contain a number of other elements, including for example, the required regulatory sequences (e.g., translation, transcription, promoters, polyadenylation sites etc), fusion partners, restriction endonuclease (cloning and subcloning) sites, stop codons preferably in all three frames, regions of complementarity for second strand priming for generating peptide libraries (preferably at the end of the stop codon region as minor deletions or insertions may occur in the peptide region), etc.

Thus, the fusion nucleic acids of the present invention may comprise a promoter. By “promoter” herein is meant nucleic acid sequences capable of initiating transcription of the fusion nucleic acid or portions thereof. Promoter may be constitutive wherein the transcription level is constant and unaffected by modulators of promoter activity. Promoter may also be inducible in that promoter activity is capable of being increased or a decreased, for example as measured by the presence of transcripts or translation products (see Walther, W. et al. (1996) J. Mol. Med. 74: 379-92). Promoter may also be cell specific, wherein the promoter is active only in particular cell types. Thus, promoter as defined herein includes sequences required for initiating and regulating the transcription level and transcription in specific cell types. Furthermore, the promoters of the present invention include within derivatives or mutant promoters, and hybrid promoters formed by combining elements of more than one promoter. Preferred promoters for expression in mammalian cells are CMV promoters and hybrid tetracycline (i.e., tetP or TRE) inducible promoters. In addition, other regulatory sequences may be included.

Generally, the retroviral vectors comprise an inducible or constitutive promoter, a first gene of interest, separation sequence, and a second gene of interest. When the retroviral vectors are used to express candidate nucleic acids or proteins, suitable reporter genes or selection genes are employed. Suitable selection genes include, but are not limited to neomycin, blastocidin, bleomycin, puromycin, and hygromycin resistance, as well as fluorescent markers such as green fluorescent protein, enzymatic markers such as β galactosidase, and surface proteins such as CD8, etc.

Generally, the regulatory nucleic acid sequences are operably linked to nucleic acids to be expressed. Nucleic acid is “operably linked” when it is placed in a functional relationship with another nucleic acid sequence. In this context, operably linked means that the transcriptional and other regulatory nucleic acids are positioned relative to a coding sequence in such a manner that transcription is initiated. Generally, this will mean that the promoter and transcriptional initiation or start sequences are positioned 5′ to the coding region. The transcriptional regulatory nucleic acid selected will be appropriate to the host cell used, as will be appreciated by those in the art. Numerous types of appropriate expression vectors, and suitable regulatory sequences, are known in the art for a variety of host cells. In addition, the fusion nucleic acids of the present invention further comprise nucleic acid sequences necessary for efficient translation of expressed fusion nucleic acids, such as translation initiation sequences and poly-adenylation signals, all of which are well known in the art.

Constructing the fusion nucleic acids of the present invention will depend in part on the separation sequence employed. The separation sequence is operably linked to the first gene of interest and second gene of interest such that the fusion nucleic acid is capable of producing separate protein products of interest. In a preferred embodiment, the separation sequence is placed in between the first and the second gene of interest. As will be appreciated by those skilled in the art, use of separation sequences based on protease recognition sites or Type 2A sequences requires that the fusion nucleic acid comprising the first gene of interest, separation sequence, and second gene of interest be in-frame. By “in-frame” herein is meant that the fusion nucleic acid encodes a continuous single polypeptide comprising the protein encoded by the first gene of interest, protein encoded by the separation sequence, and protein encoded by the second gene of interest. Standard recombinant DNA techniques may be used for placing the component nucleic acids to encode a contiguous single polypeptide. Peptide linkers may be added to the separation sequence to facilitate the separation reaction or limit structural interference of the separation sequence on the gene of interest (and vice versa). Preferred linkers are (Gly-Ser)n or (Gly)n linkers, where n is 1 or more, with n being two, three, four, five or six, although linkers of 7-10 or amino acids are also possible.

As is appreciated by those in the art, use of IRES types sequences does not require the first gene of interest, separation sequence, and second gene of interest to be in frame since IRES sequences function as internal translation initiation sites. Accordingly, fusion nucleic acids using IRES elements have the genes of interest arranged in a cistronic structure. That is, transcription of the fusion nucleic acid produces a cistronic mRNA that encodes both first gene of interest and second gene of interest with the IRES element controlling translation initiation of the downstream gene of interest. Alternatively separate IRES sequences may control the upstream and downstream gene of interest.

Preferred retroviral vectors include a vector based on the murine stem cell virus (MSCV) (see Hawley, R. G. et al. (1994) Gene Ther. 1: 136-38) and a modified MFG virus (Riviere, I. et al. (1995) Genetics 92: 6733-37), and pBABE. Other suitable vector include, among others, LRCX retroviral vector set; pSIR retroviral vector; pLEGFP-NI retroviral vector, pLAPSN retroviral vector; pLXIN retroviral vector; pLXSN retroviral vector; all of which are commercially available (e.g., Clontech). When target cells are non-proliferating (e.g., brain cells), useful viral vectors are derived from lentiviruses (Miyoshi, H. et al. (1998) J. Virol. 72: 8150-57), adenoviruses (Zheng, C. et al. (2000) Nat. Biotechnol. 18: 176-80) or alphaviruses (Ehrengruber, M. U. (1999) Proc. Natl. Acad. Sci. USA 96: 7041-46).

In addition, it is possible to configure the retroviral vector to allow inducible expression of retroviral inserts after integration of a single vector in target cells; importantly, the entire system is contained within the single retrovirus. Tet inducible retroviruses have been designed incorporating the Self-Inactivating (SIN) feature of 3′ LTR enhancer promoter retroviral deletion mutant (see Hoffman, A. et al. (1996) Proc. Natl. Acad. Sci. USA 93: 5185-90). Expression of this vector in cells is virtually undetectable in the presence of tetracycline or other active analogs (e.g., doxycyclin). However, in the absence of tetracyclin, expression is turned on within 48 hrs after induction, with uniform increased expression of the whole population of cells that harbor the inducible retrovirus, indicating that expression is regulated uniformly within the infected cell population. A similar system uses a mutated Tet DNA-binding domain such that it is bound to DNA in the presence of Tet and removed in the absence of Tet.

To ease constructing the retroviral vectors comprising the fusion nucleic acids of the present invention, the present invention also provides for retroviral cloning vectors containing multiple cloning sites, separation sequences, and/or suitable reporter/selection genes. Thus, in one aspect, the retroviral cloning vector comprises a promoter, which is either constitutive or inducible, operably linked to the gene of interest comprising a multiple cloning site (MCS). In one preferred embodiment, MCS lacks an amino acid residue capable of functioning as the initiating methionine, which allows cloning a gene of interest that has its own initiating methionine residue. Alternatively, the multiple cloning site comprises a peptide or protein coding region with its own initiating methionine for expressing proteins or peptides lacking an the initiating methionine. Additional nucleic acids encoding amino acids that increase expression of the first gene of interest (e.g., Gly or GlyGly following the initiating methionine residue) may be included in the multiple cloning site. The coding region may also comprise an indicator gene, such as lacZ, to permit identification of inserts by insertional inactivation of lacZ. In these constructs, use of a promoter controlling element capable of being active in both eukaryotes and prokaryotes will allow detecting lacZ in prokaryotes during the cloning process (see Wirtz, E. et al. (1995) Science 268: 1179-83). In either case, a separation sequence chosen from a protease based, IRES based, of Type 2A based sequence, is operably linked to the first multiple cloning site.

In another preferred embodiment, the second gene of interest of the retroviral cloning vectors may also comprise a MCS, similar to the MCS described above. This second MCS is operably linked to the separation sequence. When IRES separation sequences are used, the second gene of interest comprising the MCS may or may not contain an initiating methionine for translation initiation of a gene of interest cloned into the second MCS. For example, expression of a peptide or protein lacking its own initiating methionine requires use of an MCS with its own initiating methionine. When the separation sequence is a protease recognition site or a Type 2A sequence, an initiating methionine is not required.

As will be appreciated by those skilled in the art, various combinations of gene of interest, separation sequences, and MCS are possible in the present invention. Thus, in one aspect, the retroviral cloning vector comprises a first gene of interest comprising a first MCS, a separation sequence, and a second gene of interest comprising a second MCS. This cloning vector allows insertion of any combination of nucleic acids (i.e., genes of interest) into the first and second MCS sites to express separate peptides of interest. In another aspect, the first gene of interest comprises a reporter or selection gene while the second gene of interest comprises a MCS to allow insertion of a nucleic acid. This construct permits monitoring of expression of the protein encoded by the cloned nucleic acid. In these embodiments, the reporter or selection gene may be either distal or proximal to the promoter.

The nucleic acids for making the retroviral library of fusion nucleic acids are derived from genomic DNA or cDNA as described above. The libraries may also be directed to specific sets of encoded protein sequences such as protein-interaction domains or DNA-binding domains. These may be accomplished by use of libraries of cloned protein interaction domains, multiplex PCR of nucleic acids containing the desired polypeptide domains, or standard oligonucleotide synthesis methods.

When the nucleic acids comprise libraries of random nucleic acid sequences or random encoded peptides, these nucleic acids are preferably synthesized using known oligonucleotide synthesis techniques. These techniques include synthetic methods well known in the art and include, among others, phosphoramidite, phosphoramidate, and phosphonate chemistries (see Eckstein, Oligonucleotides and Analogues, A Practical Approach, IRL Press, Oxford University Press, 1991). Synthesis is controlled such that nucleic acids are totally random or biased random, as described above.

Preferably, the fusion nucleic acids and the library of fusion nucleic acids or candidate agents are first cloned into a viral shuttle vector to produce a library of plasmids. A typical shuttle vector is pLNCX (Clontech). The resulting plasmid library can be amplified in E. coli., purified and introduced into retroviral packaging cell lines. Suitable retroviral packaging cell lines include, but are not limited to the Bing and BOSC23 cells lines (WO 94/19478; Soneoka, Y. et al. (1985) Nucleic Acids Res. 23: 628-33; Finer, M. H. et al. (1994) Blood 83: 43-50); Phoenix packaging lines such as PhiNX-ampho; 292T+gag pol and retrovirus envelope; PA 317; and other cell lines outlined in Markowitz, D. et al. (1998) Virology 167: 400-06 (see also Markowitz, D. et al. (1998) J. Virol. 63: 1120-24; Li, K. J. et al. (1996) Proc. Natl. Acad. Sci. USA 93: 11658-63; and Kinsella, T. M. et al. (1996) Hum. Gene Ther. 7: 1405-13).

In a preferred embodiment, viruses are made by transient transfection of the cell lines referenced above. The resulting viruses can either be used directly or be used to infect another retroviral cell line for expansion of the library.

In a preferred embodiment, the library of virus particles is used to transfect packaging cell lines disclosed herein to produce a primary viral library. By “primary viral” library” herein is meant a library of virus particles comprising the fusion nucleic acids of the present invention. The production of the primary library is preferably done under conditions known in the art to reduce clone bias. The resulting primary viral library can be titred and stored, used directly to infect a target host cell line, or be used to infect another retroviral producer cell for “expansion” of the library.

Concentration of virus may be done as follows. Generally, retroviruses are titred by applying retrovirus containing supernatant onto indicator cells, such as NIH3T3 cells, and then measuring the percentage of cells expressing phenotypic consequences of infection. The concentration of virus is determined by multiplying the percentage of cells infected by the dilution factor involved, and taking into account the number of target cells available to obtain relative titre. If the retrovirus contains a reporter gene, such as lacZ, then infection, integration and expression of the recombinant virus is measured by histological staining for lacZ expression or by flow cytometry (i.e., FACS analysis). In general, retroviral titres generated from even the best of the producer cells do not exceed 10⁷ per ml unless concentrated, for example by centrifugation and ultrafiltration. However, flow-through transduction methods can provide up to a ten-fold higher infectivity by infecting cells on a porous membrane and allowing retrovirus supernatant to flow past the cells. This provides the capability of generating retroviral titres higher than those achieved by concentration (see Chuck, A. S. (1996) Hum. Gene Thre. 7: 743-50).

To obtain the secondary viral library, host cells are preferably infected with a multiplicity of infection (MOI) of 10. By secondary viral library, herein is meant a library of retroviral particles expressing the claimed fusion nucleic acids and candidate agents described herein.

As will be appreciated by those in the art, these viral libraries are used to produce the cellular libraries of the present invention. As will be appreciated by those in the art, the types of cells used in the present invention can vary widely. Basically any mammalian cells may be used, including preferred cell types from mouse, rat, primate, and human cells. As is more fully described below, cell types implicated in a wide variety of disease conditions are particularly useful, so long as a suitable screen may be designed to allow the selection of cells that exhibit an altered phenotype as a consequence of treating the cells with candidate agents. As will be appreciated by those in the art, modifications of the system by pseudotyping allows all eukaryotic cells to be used, preferably higher eukaryotes (Morgan, R. A. et al. (1993) J. Virol. 67: 4712-21; Yang, Y. et al. (1995) Hum. Gene Ther. 6: 1203-13).

Furthermore, useful are cell types capable of displaying an inducible phenotype upon expression of a first and/or second gene of interest as described herein. These cells permit screening for candidate agents altering the induced cellular phenotype. For these situations, cell lines comprising stably integrated retroviral vectors (e.g. SIN vectors) are obtained by selecting for appropriate reporter gene or selection gene expression, as described above.

The population or sample can contain a mixture of different cell types from either primary or secondary cultures although samples containing only a single cell type are preferred. For example, the sample can be from a cell line, particularly tumor cell lines, as outlined below. The cells may be in any cell phase, either synchronously or not, including M, G1, S, and G2. In a preferred embodiment, cells that are replicating or proliferating are used. This permits use of retroviral vectors for the introduction of candidate bioactive agents. Alternatively, non-replicating cells may be used, in which case adenoviral or lentiviral vectors are preferred. Preferred cell types for use in the invention include, but are not limited to, mammalian cells, including animal (e.g., rodents: mice, rats, hamsters and gerbils), primates, and human cells, particularly tumor cells such as breast, skin, lung, cervix, colorectal, leukemia, brain, etc.

Accordingly, suitable cell types include, but are not limited to, tumor cells of all types (particularly melanoma, myeloid leukemia, carcinomas of the lung, breast, ovaries, colon, kidney, prostate, pancreas, brain, testes, etc.), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as hemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

To provide those skilled in the art the tools to use the present invention, the nucleic acids and cells of the present invention are assembled into kits. The components included in the kits may comprise the retroviral vector fusion nucleic acids (e.g., retroviral cloning vectors or the retroviral libraries), enzymatic reagents for making the retroviral constructs, cells for packaging and amplification of viruses, and reagents for transfection and transduction into target cells. Alternatively, the kits contain libraries of retroviruses capable of being introduced into cells and/or contain cells already stably expressing the fusion nucleic acids (i.e., via integration of the retroviruses into the cellular chromosome).

The cells and cellular libraries comprising fusion nucleic acids of the present invention find use in screens for candidate agents producing an altered cellular phenotype. In one preferred embodiment, the method of screening cells for altered phenotype comprises (a) providing a plurality of cells, or a cellular library comprising a library of retroviral vectors, each comprising a fusion nucleic acid comprising a promoter, first gene of interest, separation site and a second gene of interest, (b) adding at least one candidate agent to the cells and (c) screening the cells for a cell with an altered phenotype. The method may further comprise (d) isolating the cell displaying the altered phenotype and (e) identifying the candidate agent responsible for the altered phenotype.

By “candidate agent” or “candidate small molecules” or “candidate expression products” or grammatical equivalents herein is meant an agent or expression product which may be tested for the ability to alter the phenotype of a cell.

Candidate agents are obtained from a wide variety of sources including libraries of synthetic or natural compounds. For example, numerous means are available for random and directed synthesis of a wide variety of organic compounds and biomolecules, including expression of randomized oligonucleotides (see for example, Gallop, M. A. et al. (1994) J. Med. Chem. 37: 1233-51; Gordon, E. M. et al. (1994) J. Med. Chem. 37:1385-401; Thompson, L. A. et al. (1996) Chem. Rev. 96: 555-600; Balkenhol, F. et al. (1996) Angew. Chem. Int. Ed. 35: 2288-337; and Gordon, E. M. et al. (1996) Acc. Chem. Res. 29: 444-54). Alternatively, libraries of natural compounds in the form of bacterial, fungal, plant, and animal extracts are available or readily produced. Additionally, natural or synthetically produced libraries and compounds are readily modified through conventional chemical, physical, and biochemical means. Known pharmacological agents may be subjected to directed or random chemical modifications such as acylation, alkylation, esterification, and amidification to produce structural analogs.

Candidate agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonly, hydroxyl, or carboxyl group, preferably at least two of them functional chemical groups. The candidate agents often comprise cyclical carbon or heterocyclic structures and or aromatic or polyaromatic structures substituted with one or more of the above functional groups. Candidate agents are also found among biomolecules including peptides, saccharides, fatty acids, steroids, purines, pyrimidines, derivatives, structural analogs, or combinations thereof. Particularly preferred are proteins, candidate drugs, and other small molecules.

The candidate agent can be pesticides, insecticides or environmental toxins; a chemical (including solvents, polymers, organic molecules, etc); therapeutic molecules (including therapeutic and abused drugs, antibiotics, etc.); biomolecules (including hormones, cytokines, proteins, lipids, carbohydrates, cellular membrane antigens and receptors (e.g., neural, hormonal, nutrient, and cell surface receptors) or their ligands, etc.); whole cells (including prokaryotic and eukaryotic (including pathogenic cells), including mammalian tumor cells); viruses (including retroviruses, herpes viruses, adenoviruses, lentiviruses, etc.); and spores (e.g., fungal, bacterial etc.).

In one preferred embodiment, the candidate agents are nucleic acids. By “candidate nucleic acids” herein is meant a nucleic acid, generally RNA when retroviral delivery vehicles are used, which can be expressed to form candidate bioactive agents; that is, the candidate nucleic acids express the candidate bioactive agents and the fusion partners, if present. In addition, the candidate nucleic acids will also generally contain enough extra sequence to effect transcription or translation, as necessary. The nucleic acid candidate agents may be naturally occurring nucleic acids, random nucleic acids, or biased random nucleic acids introduced or expressed in the subject cells. For example, they include digests of procaryotic or eukaryotic genomes as described above. In a preferred embodiment, the candidate nucleic acids are cDNA fragments generated from RNA of other organisms. As discussed above, the genomic nucleic acid and cDNA libraries can be from any number of different cells, and include libraries generated from eukaryotic and prokaryotic cell, viruses, cells infected with viruses or other pathogens, genetically altered cells etc. Preferred embodiments include nucleic acid libraries made from different individuals, such as different patients. The genomic and cDNA libraries may be complete libraries or partial libraries.

When the nucleic acids are expressed in the cells, they may or may not encode a protein as described herein. Thus, included within the candidate nucleic acids of the present invention are RNAs capable of producing an altered phenotype. Thus, in one aspect, the nucleic acid may be an antisense nucleic acid directed towards a complementary target nucleic acid. As is well known in the art, antisense nucleic acids find use in suppressing or affecting expression of various genes of pathogenic organisms or expression of cellular genes. These include suppression of oncogenes to affect the proliferative properties of transformed cells (Martiat, P. et al. (1993) Blood 81: 502-09; Daniel, R. (1995) Oncogene 10: 1607-14; Niemeyer, C. C. (1998) Cell Death Differ. 5: 440-49), modulate cell cycle (Skotz, M. et al. (1995) Cancer Res. 55: 5493-98;), inhibit proteins involved in cardiovascular disease states (Wang, H. (1999) Circ. Res. 85: 614-22) and inhibit viral pathogenesis (Lo, K. M. et al. (1992) Virology 190: 176-83; Chatterjee S. et al (1992) Science 258: 1485-88).

In another preferred embodiment, the candidate nucleic acids are nucleic acids capable of catalyzing cleavage of target nucleic acids in a sequence specific manner, preferably in the form of ribozymes. Ribozymes include among others hammerhead ribozymes, hairpin ribozymes, and hepatitis delta virus ribozymes (Tuschl, T. (1995) Curr. Opin. Struct. Biol. 5: 296-302; Usman N. (1996) Curr Opin Struct Biol 6: 527-33; Chowrira B. M. et al. (1991) Biochemistry 30: 8518-22; Perrotta A. T. et al. (1992) Biochemistry 3: 16-21). As with antisense nucleic acids, nucleic acids catalyzing cleavage of target nucleic acids may be directed to a variety of expressed nucleic acids, including those from pathogenic organisms or cellular genes (see for example, Jackson, W. H. et al. (1998) Biochem. Biophys. Res. Commun. 245: 81-84).

Another preferred embodiment of candidate nucleic acids are double stranded RNA capable of inducing RNA interference or RNAi (Bosher, J. M. et al. (2000) Nat. Cell Biol. 2: E31-36). Introducing double stranded RNA can trigger specific degradation of homologous RNA sequences, generally within the region of identity of the dsRNA (Zamore, P. D. et. al. (1997) Cell 101: 25-33). This provides a basis for silencing expression of genes, thus permitting a method for altering the phenotype of cells. The dsRNA may comprise synthetic RNA made either by known chemical synthetic methods or by in vitro transcription of nucleic acid templates carrying promoters (e.g., T7 or SP6 promoters). Alternatively, the dsRNAs are expressed in vivo, preferably by expression of palindromic fusion nucleic acids, that allow facile formation of dsRNA in the form of a hairpin when expressed in the cell. The double strand regions of the hairpin RNA are generally about 10-500 basepairs or more, preferably 15-200 basepairs, and most preferably 20-100 basepairs.

When the candidate nucleic acids are random nucleic acids, they are randomized, either fully randomized or they are biased in their randomization, e.g., in nucleotide/residue frequency generally or per position. As defined above, by “randomized” or grammatical equivalents herein is meant that each nucleic acid and peptide consists of essentially random nucleotides and amino acids, respectively. As is more fully described below, the candidate nucleic acids are chemically synthesized, and thus may incorporate any nucleotide at any position. In the expressed random nucleic acid, at least 10, preferably at least 12, more preferably at least 15, most preferably at least 21 nucleotide positions need to be randomized, with more preferable if the randomization is less than perfect. The candidate nucleic acids may also comprise nucleic acid analogs as described above.

In another aspect, a preferred embodiment of candidate agents are proteins and peptides. In one preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way, libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

Candidate agents may encompass a variety of peptidic agents. These include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs such as phenytoin, primidone, carbariezepin, ethosuximide, valproic acid, and phenobarbitol), cardioactive drugs (digoxin, lidocaine, procainamide, and disopyramide), bronchodilators (theophylline), antibiotics (e.g., chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g., respiratory syncytial virus, mumps virus, measles virus), adenoviruses, rhinoviruses, coronaviruses, reoviruses, togaviruses (e.g. rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C.perfringens; Cornyebacterium, e.g. C. diphtheriae; Streptococcus, S. pyogenes, S. pneumoniae; Staphylococcus, e.g., S. aureus; Haemophilus, e.g. H. influenzae; Neisseria, e.g. N. meningitidis, N. gonorrhoeae; Yersinia, e.g. G. lambliaY. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphatase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cortisol, estradiol, follicle stimulating hormone (FSH), thyroid-stimulating hormone (TSH), luteinizing hormone (LH), progesterone, testosterone,; and (4) other proteins (including α-fetoprotein, carcinoembryonic antigen CEA.

In a preferred embodiment, the candidate bioactive agents are peptides from about 5 to about 30 amino acids, with from about 5 to about 20 amino acids being preferred, and from about 7 to about 15 being particularly preferred. The peptides may be digests of naturally occurring proteins, random peptides, or “biased” random peptides as set forth above. Since generally these random peptides are chemically synthesized or encoded by chemically synthesized nucleic acids, they may incorporate any amino acid at any position. The synthetic process can be designed to generate randomized proteins, to allow the formation of all or most of the possible combinations over the length of the sequence, thus forming a library of randomized candidate bioactive proteinaceous agents. As explained above, in one embodiment, the library is fully randomized, with no sequence preferences or constants at any position. In a preferred embodiment, the library is biased. That is, some positions within the sequence are either held constant, or are selected from a limited number of possibilities.

Accordingly, in a preferred embodiment, the candidate bioactive agents are encoded by candidate nucleic acids. For an encoded peptide library, the candidate nucleic acid generally contain cloning sites which are placed to allow in-frame expression of the randomized peptides, and any fusion partners, if present, such as presentation structures. For example, when presentation structures are used, the presentation structure will generally contain the initiating ATG, as a part of the parent vector.

For candidate nucleic acid agents, the candidate nucleic acids may be expressed from vectors well known in the art, including retroviral vectors. Thus, when RNAs are expressed, vectors expressing the candidate nucleic acids may be generally constructed with an internal promoter (e.g., CMV promoter), tRNA promoter, cell specific promoter, or hybrid promoters designed for immediate and appropriate expression of the RNA structure at the initiation site of RNA synthesis. The RNA may be expressed anti-sense to the direction of retroviral synthesis and is terminated as known, for example with an orientation specific terminator sequence. Interference from upstream transcription is alleviated in the target cell with the self-inactivation deletion (SIN), a known feature of certain retroviral expression systems.

Accordingly, in one preferred embodiment, the retroviral vectors expressing the candidate agents may comprise fusion nucleic acids of the present invention. In one embodiment, the fusion nucleic acid encoding the candidate peptides comprise at least one of the gene of interest in the fusion nucleic acid. That is, the first or second gene of interest comprises a nucleic acid encoding random peptides. The presence of a separation sequence and a second gene of interest comprising a reporter or selection gene allows identification of cells expressing the candidate peptides of interest without affecting the activity of the peptide itself. In another embodiment, the first and second gene of interest comprise nucleic acids encoding different candidate peptide agents, thus permitting expression of multiple peptide candidate agents within a single cell.

In a preferred embodiment, a library of candidate bioactive agents are used. As discussed above, the library should be sufficiently structurally diverse population to effect a probabilistically sufficient range to provide one or more nucleic acids or peptide products which has the desired properties such as binding to protein interaction domains or producing a desired cellular response. Thus, preferred methods maximize library size and diversity.

The candidate agents are combined or added to a cell, a population of cells, or a plurality of cells. By “population of cells” or “plurailty of cells” herein is meant at least two cells with at least about 10⁵ being preferred, at least about 10⁶ being particularly preferred, and at least about 10⁷, 10⁸ and 10⁹ being especially preferred.

The candidate agents and the cells are combined. As will be appreciated by those in the art, this may be accomplished in any number of ways, including adding the candidate agents to the surface of the cells, to the media containing the cells, or to a surface on which cells are growing or are in contact with; adding agents into the cells, for example by using vectors that will introduce the agents into the cells, especially when the agents are nucleic acids or proteins.

Since the cells may comprise a first fusion nucleic acid that expresses genes of interest that allow detecting or induces a phenotype of a cell and a second fusion nucleic acid that expresses candidate agents, the present invention provides for cells containing a plurality of fusion nucleic acids of the present invention. Use of distinguishable reporter proteins for the first and the second fusion nucleic acid provides a way of distinguishing expression of the two fusion nucleic acids in the cell. Additional fusion nucleic acids may be introduced into the cells to express other genes of interest. In this way, any number of genes of interest, including candidate nucleic acids, may be expressed within a single cell.

In a preferred embodiment, the candidate agents are either proteins or nucleic acids that are introduced into the cells. By “introduced into” or grammatical equivalents herein is meant that the nucleic acids enter the cells, especially in a manner suitable for subsequent expression of the nucleic acid. The method of introduction is largely dictated by the targeted cell type. Exemplary methods include CaPO₄ transfection, DEAE dextran transfection, liposome fusion, lipofectin®, electroporation, viral infection, biolistic particle bombardment etc. The candidate nucleic acids may stably integrate into the genome of the host cell (e.g., by retroviral integration), or may exist either transiently or stably in the cytoplasm (e.g., through the use of traditional plasmids utilizing standard regulatory sequences, selection markers, promoters, etc.). Since many pharmaceutically important screens require human or model mammalian cell targets, retroviral vectors capable of transfecting such targets are preferred.

In a preferred embodiment, the candidate bioactive agents are either nucleic acids or proteins (proteins in this context includes proteins, oligopeptides, and peptides) that are introduced into the host cells using vectors, including viral vectors. Vectors for expressing nucleic acids and proteins are well known in the art. The choice of the vector, preferably a viral vector, will depend on the cell type. When cells are replicating, retroviral vectors are used. When the cells are not replicating, for example when arrested in one of the growth phases, other viral vectors are suitable, such as lentiviral and adenoviral vectors.

In a preferred embodiment, the candidate bioactive agents are either nucleic acids or proteins that are introduced into the host cells using retroviral vectors, as is generally outlined in PCT US 97/01019, PCT US97/01048, and U.S. Pat. No. 6,153,380, all of which are expressly incorporated by reference. Generally, a library is generated using a retroviral vector backbone. For generating nucleic acid or peptide libraries, standard oligonucleotide synthesis may be done to generate the candidate nucleic acid using techniques well known in the art. After generating the nucleic acid library, the library is cloned into a first primer, which serves as a cassette for insertion into the retroviral construct. The first primer generally contains additional elements, including for example, the required regulatory sequences (e.g., translation, transcription, promoters, etc.) fusion partners, restriction endonuclease sites, stop codons, regions of complementarity for second strand priming, etc.

A second primer is then added, which generally consists of some or all of the complementarity region to prime the first primer and optional sequences necessary to a second unique restriction site for purposes of subcloning. Extension with DNA polymerase results in double stranded oligonucleotides, which are then cleaved with appropriate restriction endonucleases and subcloned into the target retroviral vectors.

The retroviral vectors may include selectable marker genes; promoters driving expression of a second gene, placed in sense or anti-sense relative to the 5′ LTR; CRU5 (a synthetic LTR), tetracycline regulation elements in SIN; cell specific promoters, etc. In addition, the retroviruses may include inducible and constitutive promoters for the expression of the candidate agent. For example, there are situations wherein it is necessary to induce peptide expression only during certain phases of the selection process, such as during particular periods of the cell cycle. A large number of constitutive and promoters are well known.

Any number of suitable retroviral vectors may be used. In one aspect, preferred vectors include those based on murine stem cell virus (MSCV) (Hawley, et al. (1994) Gene Therapy 1: 136), a modified MFG virus (Reivere et al. (1995) Genetics 92: 6733), pBABE, and others described above. Well suited retroviral transfection systems are described in Mann et al, supra; Pear et al. (1993) Proc. Natl. Acad. Sci. USA 90: 8392-96; Kitamura, et al. Human Gene Ther. 7: 1405-1413; Hofmann, et al Proc. Natl Acad. Sci. USA 93: 5185-90; Choate et (1996) Human Gene Ther 7: 2247; WO 94/19478; PCT US97/01019, and references cited therein, all of which are incorporated by reference.

In a preferred embodiment, bioactive candidate agents are linked to a fusion partner, as described above. In one aspect, combinations of fusion partners are used. Any number of combinations of presentation structures, targeting sequences, rescue sequences, and stability sequences may be used with or without linker sequences. Thus, candidate agents, which include these components, may be used to generate a library of fragments, each containing a different random nucleotide sequence that may encode a different peptide. The ligation products are then transformed into bacteria, such as E. coli. and DNA is prepared from the resulting library, as is generally outlined in Kitamura, T. (1995) Proc. Natl. Acad. Sci. USA 92: 9146-50 and as fully discussed above.

In a preferred embodiment, when the candidate agent is introduced into the cells using a viral vector, the candidate agent is linked to a detectable molecule, and the methods of the invention include at least one expression assay. Thus, the detectable molecule may comprise reporter and selection genes as described herein. In one preferred embodiment, the detectable molecule is distinguishable from that expressed by the fusion nucleic acid expressing the plurality of genes of interest. An expression assay is an assay that allows the determination of whether a candidate bioactive agent has been expressed, i.e., whether a candidate peptide agent is present in the cell. Thus, by linking the expression of a candidate agent to the expression of a detectable molecule such as a label, the presence or absence of the candidate peptide agent may be determined. Accordingly, in this embodiment, the candidate agent is operably linked to a detectable molecule. Generally, this is done by creating a fusion nucleic acid. The fusion nucleic acid comprises a first nucleic acid encoding the candidate bioactive agent (which can include fusion partners, as outlined above), and a second nucleic acid encoding a detectable molecule. The terms “first” and “second” are not meant to confer an orientation of the sequences with respect to 5′-3′ orientation of the fusion nucleic acid. For example, assuming a 5′-3′ orientation of the fusion sequence, the first nucleic acid may be located either 5′ to the second nucleic acid, or 3′ to the second nucleic acid. Preferred detectable molecules in this embodiment include, but are not limited to, various fluorescent proteins and their variants described above, including A. victoria GFP, Renilla muelleri GFP, Renilla reniformis GFP, Ptilosarcus gurneyi GFP, Anemonia majano fluorescent protein, Zoanthus fluorescent proteins, Clavularia fluorescent protein, Discosoma fluorescent protein, YFP, BFP and RFP.

In general, the candidate agents are added to the cells under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high throughput screening. Typically between 0.1 and 1 hour will be sufficient. Excess reagent is generally removed or washed away.

A variety of other reagents may be included in the assays. These include reagents like salts, neutral proteins, e.g., albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for detection. Washing or rinsing the cells will be done as will be appreciated by those in the art at different times, and may include the use of filtration and centrifugation. When second labeling moieties (also referred to herein as “secondary labels”) are used, they are preferably added after excess non-bound target molecules are removed, in order to reduce non-specific binding; however, under some circumstances, all the components may be added simultaneously.

As will be appreciated by those in the art, the type of cells used in the present invention can vary widely. Basically, the screen may use any mammalian cells in which the library of retroviral vectors comprising the fusion nucleic acids of the present invention are made. Particularly preferred are cells from mouse, rat, primate and human cells, although as will be appreciated by those in the art, modifications of the system by pseudotyping allows all eukaryotic cells to be used, preferably higher eukaryotes (Morgan, R. A. et al. (1993) J. Virol. 67: 4712-21; Yang, Y. et al. (1995) Hum. Gene Ther. 6: 1203-13).

As is more fully described below, a screen will be set up such that the cells exhibit a selectable phenotype in the presence of a candidate agent. Thus, cell types implicated in a wide variety of disease conditions are particularly useful, so long as a suitable screen may be designed to allow the selection of cells that exhibit an altered phenotype as a consequence of the presence of a candidate bioactive agent within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as hemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In one embodiment, the cells may be genetically engineered; that is, contain exogenous nucleic acids, for example, to contain target molecules.

In a preferred embodiment, a first plurality of cells is screened. That is, the cells into which the candidate nucleic acids are introduced are screened for an altered phenotype. Thus, in this embodiment, the effect of the bioactive candidate agent is seen in the same cells in which it is made; i.e., an autocrine effect.

By a “plurality of cells” herein is meant roughly from about 10³ cells to 10⁸ or 10⁹, with from 10⁶ to 10⁸ being preferred. This plurality of cells comprises a cellular library, wherein generally each cell within the library contains a member of the library of candidate agents, including member of a retroviral molecular library (i.e., a different candidate nucleic acid), although as will be appreciated by those in the art, some cells within the library may not contain a candidate agent, and some may contain more than one. For example, when methods other than retroviral infection are used to introduce candidate nucleic acids into a plurality of cells, the distribution of candidate nucleic acids within the individual cell members of the cellular library may vary widely, as it is generally difficult to control the number of nucleic acids which enter a cell during electroporation, etc.

In a preferred embodiment, the candidate agents are introduced into a first plurality of cells, and the effect of the candidate bioactive agents is screened in a second or third plurality of cells, different from the first plurality of cells, i.e., generally a different cell type. That is, the effect of the bioactive agents is due to an extracellular effect on a second cell; i.e., an endocrine or paracrine effect. This is done using standard techniques. The first plurality of cells may be grown in or on one media, and the media is allowed to touch a second plurality of cells, and the effect measured. Alternatively, there may be direct contact between the cells. Thus, contacting is functional contact, and includes both direct and indirect. In this embodiment, the first plurality of cells may or may not be screened.

If necessary, the cells are treated to conditions suitable for expression of the candidate nucleic acid; for example, when inducible promoter are used to express the candidate agents. Expression of the candidate agents results in functional contact of the candidate agent and the cell. Thus, in one preferred embodiment, the methods of the present invention comprise introducing candidate nucleic acids into a plurality of cells to form a cellular library. The plurality of cells is then screened, as is more fully outlined below, for a cell exhibiting an altered phenotype. The altered phenotype is due to the presence of a candidate bioactive agent.

By “altered phenotype” or “changed physiology” or “dominant effect” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptibility, latency, adhesion, and uptake of viruses and bacterial pathogens; etc. By “capable of altering the phenotype” herein is meant that the candidate agent can change the phenotype of the cell in some detectable and/or measurable way.

The altered phenotype may be detected in a wide variety of ways, as is described more fully below, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc. In some cases, as is more fully described herein, the altered phenotype is detected in the cell in which the candidate agent (e.g., genomic DNA, cDNA, or randomized nucleic acid) was introduced; in other embodiments, the altered phenotype is detected in a second cell which is responding to some molecular signal from the first cell.

In a preferred embodiment, once a cell with an altered phenotype is detected, the cell is isolated from the plurality which do not have altered phenotypes. Isolation of the altered cell may be done in any number of ways, as is known in the art, and will in some instances depend on the assay or screen. Suitable isolation techniques include, but are not limited to, FACS, lysis selection using complement, cell cloning, scanning by Fluorimager, expression of a “survival” protein, induced expression of a cell surface protein or other molecule that can be rendered fluorescent or taggable for physical isolation; expression of an enzyme that changes a non-fluorescent molecule to a fluorescent one; overgrowth against a background of no or slow growth; death of cells and isolation of DNA or other cell vitality indicator dyes, etc.

In a preferred embodiment, the candidate nucleic acid and/or the bioactive agent is isolated from the positive cell. In one preferred embodiment, primers complementary to DNA regions common to the retroviral constructs, or to specific components of the library such as a rescue sequence as defined above, are used to “rescue” the unique candidate agent. Alternatively, the bioactive candidate agent is isolated using a rescue sequence. Thus, for example, rescue sequences comprising epitope tags or purification sequences may be used to pull out the bioactive candidate agent, for example by immunoprecipitation or affinity columns. In some instances, as is outlined below, this may also pull out the primary target molecule if there is a sufficiently strong binding interaction between the bioactive agent and the target molecule. Alternatively, the peptide may be detected using mass spectroscopy.

Once rescued, the sequence of the candidate agent and/or bioactive nucleic acid is determined. This information can then be used in a number of ways.

In a preferred embodiment, the candidate agent is resynthesized and reintroduced into the target cells, to verify the effect. This may be done using retroviruses, or alternatively using fusions to the HIV-1 Tat protein and its analogs and related proteins, which allows very high uptake into target cells (see for example, Fawell, S. et al.(1994) Proc. Natl. Acad. Sci. USA 91: 664-68; Frankel, A. D. et al.(1988) Cell 55: 1189-93; Savion, N. et al. (1981) J. Biol. Chem. 256: 1149-54; Derossi, D. et al. (1994) J. Biol. Chem. 269:10444-50; and Baldin, V. et al. (1990) EMBO J. 9: 1511-17, all of which are incorporated by reference).

In a preferred embodiment, if the candidate agent is a nucleic acid or peptide, its sequence is used to generate more candidate bioactive agents. For example, the sequence of the candidate agent may be the basis of a second round of (e.g., biased) randomization to develop other candidate agents with increased or altered activities. Alternatively, the second round of randomization may change the affinity of the candidate agent. Furthermore, it may be desirable to put the identified sequence of the random region of the candidate agent into other presentation structures, or to alter the sequence of the constant region of the presentation structure, in order to change the conformation/shape of the candidate agent. It may also be desirable to “walk” around a potential binding site, in a manner similar to the mutagenesis of a binding pocket, by keeping one end of the ligand region constant and randomizing the other end to shift the binding of the peptide around.

In a preferred embodiment, either the candidate agent or the candidate nucleic acid encoding it is used to identify target molecules. As will be appreciated by those in the art, there may be primary target molecules, to which the candidate agent binds or acts upon directly, and there may be secondary target molecules, which may be part of the signaling pathway affected by the bioactive agent; these might be termed “validated targets”.

In a preferred embodiment, the bioactive agent is used to pull out target molecules. For example, as outlined herein, if the target molecules are proteins, the use of epitope tags or purification sequences can allow the purification of primary target molecules via biochemical means (co-immunoprecipitation, affinity columns, etc.). Alternatively, the peptide, when expressed in bacteria and purified, can be used as a probe against a bacterial cDNA expression library made from mRNA of the target cell type. Alternatively, peptides can be used as “bait” in either yeast or mammalian two or three hybrid systems. Such interaction cloning approaches have been very useful in isolating DNA-binding proteins and other interacting protein components. The peptide(s) can be combined with other pharmacologic activators to study the epistatic relationships of signal transduction pathways in question. It is also possible to synthetically prepare labeled peptide candidate agent and use it to screen a cDNA library expressed in bacteria or in a bacteriophage for those expressed cDNAs which bind the peptide. Furthermore, it is also possible that one could express cDNAs via retroviral libraries to “complement” the effect induced by the peptide. In such a strategy, the peptide would be required to be stoichiometrically titrating away some important factor for a specific signaling pathway. If this molecule or activity is replenished by overexpression of a cDNA from within a cDNA library, then one can clone the target molecule. Similarly, cDNAs cloned by any of the above bacteriophage, bacterial, or yeast systems can be reintroduced into mammalian cells to confirm that they act to complement function in the system the peptide acts upon.

Once primary target molecules have been identified, secondary target molecules may be identified in the same manner, using the primary target as the “bait”. In this way, signaling pathways may be elucidated. Similarly, bioactive agents specific for secondary target molecules may also be discovered in order to identify a number of bioactive agents that act on a single pathway, for example for developing combination therapies.

The methods of the present invention may be useful for screening a large number of cell types under a wide variety of conditions. Generally, the host cells are cells that are involved in disease states, and they are tested or screened under conditions that normally result in undesirable consequences on the cells. When a suitable bioactive candidate agent is found, the undesirable effect may be reduced or eliminated. Alternatively, normally desirable consequences may be reduced or eliminated, with an eye towards elucidating the cellular mechanisms associated with the disease state or signaling pathway.

Accordingly, the compositions and methods described herein are useful in a variety of applications. In one preferred embodiment, the retroviral fusion constructs are used to screen for modulators of promoter activity. By “modulation” of promoter activity herein is meant increase or decrease in transcription of the fusion nucleic acid regulated by the promoter of interest. A variety of promoters are amenable to analysis. These include, for example, IL-4 inducible E promoter, myc regulated promoters, NF-kB regulated promoters, promoters regulating HIV viral gene expression, and promoters regulating cell cycle genes. Preferred are promoters regulating expression of signal transduction proteins, cell cycle regulatory proteins, oncogenes, or promoters which are themselves regulated by signal transduction pathways, cell cycle regulators, or other aspects of cell regulatory networks. The first gene of interest may comprise a first reporter protein while the second gene of interest comprises a second reporter protein, thus providing two basis for measuring transcription levels. The candidate agents are introduced or combined with cells containing the retroviral fusion constructs. If the promoter is inducible, promoter is induced with appropriate stimulus or effector. Alternatively, the promoter is induced prior to addition of the candidate bioactive agents, or simultaneously. For example, for the IL-4 inducible E promoter, addition of cytokines IL-4 or IL-13 to the cells at concentration of not less than 5 units/ml and at a preferred concentration of 200 units/ml can induce transcription of the E promoter.

The presence or absence of the reporter gene product is then detected. This may be done in a number of ways, as will be appreciated by those in the art, and will depend on the reporter or selection gene. For example, cells expressing a reporter gene, such as GFP, can be distinguished from those not expressing the gene, and preferably sorted based on expression levels. Similarly, cells expressing a death gene will die, leaving mostly cells that have inhibited promoter activity. Thus, for stringent selection of promoter regulators, the fusion nucleic acid may comprise a promoter, a reporter gene, a separation sequence, and a selection gene. The reporter gene, such as GFP, allows selection of cells expressing the reporter while the selection gene provides an additional basis for selecting cells. Cells that express the reporter and selection gene are selected from those that do not, which may be done by FACS, cell cloning, growth under drug resistance, enhanced growth etc. For example, if the selection gene is a thymidine kinase (i.e., a death gene), the cells can be selected based on killing by gangcyclovir since TK activity is needed for gangcyclovir toxicity. Alternatively, the selection gene may encode the HBEGF protein and the killing initiated by adding diptheria toxin. Candidate agents that repress promoter activity are readily identified by selecting for cells that show resistance to cell death and lack GFP reporter gene expression. The presence of a separation sequence, such as Type 2A, permits expression of both reporter and selection genes from a single transcript, thus providing a sensitive indicator of promoter activity.

In a preferred embodiment, the presence or absence of the reporter gene is determined using a fluorescence activated cell sorter (FACS). In general, the expression of the reporter gene comprising a label or indirect label is optimized to allow for efficient enrichment by FACS. Thus, for example 10 to 1000 fluors per sorting event (i.e. per cell) allows efficient sorting, with from about 100-1000 being preferred, and from about 500-1000 being especially preferred. The presence of two reporters genes provides for higher sensitive detections as compared to expression of a single reporter gene. This is achieved by either increasing the number of fluors per cell or by providing two independent basis for selection.

In a preferred embodiment, the cell are sorted at high speeds but at speeds that preserve viability of the cells. Sorting speeds may be approximately 5000 sorting events/s, with about 5000-10,000 sorting events/s being preferred, and greater than 25,000 sorting events/s being especially preferred. Sorting speed are selected according to the sensitivity of the cells to shear forces, which may be determined by those skilled in the art (e.g., by determining viability of sorted cells).

In another aspect, when candidate agents are peptides expressed by the retroviral fusion constructs of the present invention, the second gene of interest may comprise a reporter gene distinguishable from the reporter gene used to measure promoter activity. The use of two distinguishable reporters allows selection of cells expressing both reporter and candidate peptide.

Alternatively, in another aspect, the second gene of interest on the fusion nucleic acid expressing the candidate peptide agent may comprise a regulator of the subject promoter. This construct provides a simplified manner of expressing both the candidate agent and the promoter regulator, which then provides a basis for identifying candidate agents that act directly or indirectly on the expressed transcriptional regulator.

In another preferred embodiment, the retroviral vectors and cellular libraries of the present invention are useful in identifying candidate agents affecting proteases involved in pathogenesis. As is well known in the art, viral pathogenesis and cellular physiology is regulated by the activity of various proteases. For example, HIV protease acts on the gag-pol precursor to generate the mature polymerase required for replication of the virus. This viral protease is a prime target for protease inhibitor based anti-HIV therapies. Other viral proteases are involved in processing of viral polyproteins, which are necessary to produce mature, infectious viral particles. In regards to cellular regulation, caspases comprise a family of proteases involved in activating cell death pathways. Lysozomal proteases, such as the cathepsin family, are involved in processing of proteins in the lysozomes and are believed to play a role in metastasis of tumor cells. Extracellular proteases, including metalloproteases act on extracellular matrix to regulate cell-cell interactions. Increased activity of metalloproteases are thought to reduce contact inhibition between cells, thus promoting tumor cell growth and metastasis. Tissue inhibitors of extracellular matrix metalloproteases are frequently deleted in certain cancers, such as breast cancer, suggesting that they act to create metastatic potential. Consequently, numerous proteases serve as important targets for therapeutic agents.

Accordingly, in one embodiment, the retroviral vectors of the present invention comprise a fusion nucleic acid comprising a separation sequence recognized by a protease, such as the HIV protease or caspase. The first gene of interest and the second gene of interest encode distinguishable reporter molecules. These retroviral vectors are introduced into cells, preferably to form a stable cell line expressing the fusion nucleic acid. The cell lines express the protease being examined or the protease is introduced exogenously, for example by viral infection or by transfection with a nucleic acid construct expressing the protease. When stable cellular expression of the protease is difficult, the protease may be included in the fusion nucleic acids of the present invention through addition of a second separating sequence and the additional gene of interest comprising the protease. Thus the fusion nucleic acid contains the complete protease, protease recognition site and the appropriate reporter molecules to permit detection of candidate agents acting on the protease. Preferably, the protease is expressed in the cell through an inducible promoter. Cells are then treated with candidate agents and analyzed for agents that prevent protease activity by preventing production of separate protein products of the genes of interest.

In one preferred embodiment of a protease assay, the first gene of interest comprises a cyan GFP, which is linked via a specific protease recognition site to a second gene of interest, a blue GFP capable of fluorescence resonance energy transfer (FRET). The cells are then contacted with candidate agents and assayed for those agents that inhibit protease action on the separation sequence. Inhibitors will prevent separation of the GFP molecules and allow increase in the FRET signal. In this way, candidate agents are identified that have potential anti-viral or anti-pathogenic activity by blocking protease activity. As an alternative to the FRET based assay, the first reporter gene may be targeted to a cellular location distinguishable from the cellular localization of the second reporter gene. In the absence of a separation reaction, the fusion protein comprising the first reporter protein, protease recognition site, and second reporter protein is directed predominantly to the cellular location of the first reporter protein. For example, the first reporter protein could be targeted to the plasma membrane while the second reporter protein has nuclear localization sequences. In the absence of protease activity, the fusion protein is predominantly localized to the plasma membrane. In the presence of protease, the two reporters are separated, thus allowing the second reporter to properly localize to the nucleus. The redistribution of the reporter protein resulting from protease action provides a measure of protease activity within the cell. In addition, if the reporter protein produces a dominant effect on the cell when properly localized to a subcellular compartment, the display of a dominant effect on the cell provides a useful indicator of protease activity.

In another embodiment for identifying protease inhibitors, the first gene of interest may be a DNA binding domain while the second gene of interest is a transcriptional activation domain. The sequence linking the DNA binding domain and the transcription activator domain comprises the protease recognition site. In the absence of protease, the fusion nucleic acid produces a fusion protein capable of activating transcription of a second promoter/reporter gene construct whose expression is regulated by the fusion protein. This reporter construct is stably integrated in the cell or is introduced into the cell by transfection or viral delivery. Upon expression of the protease under study, separation of the DNA binding domain and transcriptional activation domain occurs, thereby reducing or eliminating transcription of the second promoter/reporter gene construct. Candidate agents are then screened for protease inhibiting activity by detecting transcription of the reporter gene. This assay allows high throughput screens to identity protease inhibitors, for example inhibitors of HIV proteases, including variant proteases resistant to protease inhibitor based anti HIV therapy.

Since many proteases are present extracellularly, the fusion nucleic acids of the present invention may comprise a secretory sequence operably linked to an upstream first gene of interest, preferably encoding a first reporter protein, while a transmembrane anchoring domain sequence is inserted or fused to a downstream second gene of interest, which encodes a second reporter protein. The separator sequence is a peptide region recognized by a extracellular protease, such as a metalloprotease. Upon expression of the fusion nucleic acid in a cell, a fused polypeptide comprising the first protein of interest, protease recognition site, and the second protein of interest is displayed on the cell surface and anchored to the cell membrane via the transmembrane domain. Exposure of the cells to extracellular protease, for example by contact with co-cultured cells expressing the extracellular protease, results in release of the first reporter protein, which is conveniently detected in the cellular medium. Candidate agents are added to cells displaying the fusion protein to screen for inhibitors of the extracellular protease. Since metalloproteases and other extracellular proteases are believed to affect the metastatic potential of tumor cells, this type of approach provides a screening method for identifying potential anti-metastatic agents.

In another preferred embodiment, the retroviral vectors comprise a fusion nucleic acid in which the separation site is an IRES element derived from a pathogenic virus, such as hepatitis C virus (HCV) IRES, or a cellular IRES element responsible for expression of gene products involved in cellular disease states. Thus, a fusion nucleic acid construct may comprise a first gene of interest comprising a first reporter/selection gene, an HCV IRES element, and a second gene of interest comprising a second reporter/selection gene. In this embodiment, the IRES element preferably regulates expression of the downstream gene of interest. Cells expressing the fusion nucleic acids are selectable based on expression of both first and second genes of interest. The genes of interest may be distinguishable reporter and/or selection genes or genes of interest distinguished by their targeting to different cellular compartments. Candidate agents are introduced into cells and screened for their ability to inhibit IRES dependent expression of the second reporter/selection gene. The first reporter/selection gene serves as a useful monitor for expression of the fusion nucleic acid and for distinguishing inhibitory effects of candidate agents on transcription as compared to translation. Candidate agents identified using these assays will provide a way of identifying cellular or viral target molecules mediating IRES dependent translation initiation events. It will provide a basis for developing therapeutic agents effective against viruses and disease states dependent on IRES mediated regulation.

Similarly, another aspect of the present invention comprises fusion nucleic acids in which the separation site is a Type 2A sequence from a pathogenic virus or a Type 2A sequence mediating expression of a gene product responsible for a cellular disease state. In assays similar to those described above, the fusion nucleic acids comprise a first reporter/selection gene, a Type 2A separation sequence, and a second reporter/selection gene. In this construct, the fusion nucleic acid expresses separate reporter/selection proteins encoded by the first and second genes of interest. These expressing cells are treated with candidate agents to identify inhibitors of the 2A separating activity as indicated by the production of unseparated proteins encoded by the first and second genes of interest. For example, the assays may incorporate use of GFP based FRET, whereby inhibition of 2A separation activity results in increased FRET signal arising from retention of linkage between GFP reporter molecules. If the assay uses cellular localization of the reporter proteins as the basis to detect separate reporter/selection proteins, inhibition of 2A separating activity will result in altered cellular localization of the reporter/selection genes. Alternatively, when the first and second reporter genes encode a DNA binding domain and a transcriptional activation domain, respectively, inhibiting the Type 2A separation activity results in expression of a functional transcriptional regulator capable of increasing expression of a second promoter/reporter construct controlled by the transcriptional regulator.

While the discussions above relate to inhibitors of the separation reactions, the fusion nucleic acids and the described assays are equally applicable for identifying activators of separation reactions.

In another preferred embodiment, the present invention finds use in screening for cells with altered exocytosis phenotypes. By “alternation” or “modulation” in relation to excocytosis is meant a decrease or increase in amount or frequency of exocytosis in one cell compared to another cell or in the same cell under different conditions. Often mediated by specialized cells, exocytosis is vital for a variety of cellular processes, including neurotransmitter release by neurons, hormone release by adrenal chromaffin cells (e.g., adrenaline) and pancreatic β-cells (e.g., insulin), and histamine release by mast cells.

Disorders involving exocytosis are numerous. For example, inflammatory immune response mediated by mast cells leads to a variety of disorders, including asthma and allergies. Therapy for allergy remains limited to blocking mediators released by mast cells (e.g., antihistamines) and non-specific anti-inflammatory agents, such as steroids and mast cell stabilizers. These treatments are only marginally effective in alleviating the symptoms of allergy. To identify cellular targets for drug design or candidate effectors of exocytosis, the retroviral vectors of the present invention comprising libraries of candidate agents may be introduced into appropriate cells, for example mast cells, and selected for modulation of exocytosis by assaying for changes in cellular exocytosis properties. These cells are stimulated with appropriate inducer if exocytosis is triggered by an inducing signal.

Assays for changes in exocytosis may comprise sorting cells in a fluorescence cells sorter (FACS) by measuring alterations of various exocytosis indicators, such as light scattering, fluorescent dye uptake, fluorescent dye release, granule release, and quantity of granule specific proteins (as provided in application U.S. Ser. No. 09/293,670, hereby expressly incorporated by reference). Selection based on combinations of indicators reduces background and increases specificity of the sorting assay.

Exocytosis assays based on changes in the cell's light scattering properties, including use of forward and side scatter properties of the cells, are indicative of the size, shape, and granule content of the cell. Multiparameter FACS selections based on light scattering properties of cells are well known in the art (see Paretti, M. et al. (1990) J. Pharmacol. Methods 23: 187-94; Hide, I. et al. (1993) J. Cell Biol. 123: 585-93).

Assays based on uptake of fluorescent dyes reflect the coupling of exocytosis and endocytosis in which endocytosis levels indirectly reflect exocytosis levels since the cell attempts to maintain cell volume and membrane integrity as the amount of cell membrane rapidly changes when secretory vesicles fuse with the cell membrane. Preferred fluorescent dyes include styryl dyes, such as FM1-43, FM4-64, FM14-68, FM2-10, FM4-84, FM1-84, FM14-27, FM14-29, FM3-25, FM3-14, FM5-55, RH414, FM6-55, FM10-75, FM1-81, FM9-49, FM4-95, FM4-59, FM9-40, and combinations thereof. Styryl dyes such as FM1-43 are only weakly fluorescent in water but very fluorescent when associated with a membrane, such that dye uptake by endocytosis is readily discernable (Betz, et al. (1996) Current Opinion in Neurobiology, 6:365-371; Molecular Probes, Inc., Eugene, Oregon, “Handbook of Fluorescent Probes and Research Chemicals”, 6th Edition, 1996, particularly, Chapter 17, and more particularly, Section 2 of Chapter 17, (including referenced related chapters), hereby incorporated herein by reference). Useful solution dye concentrations are about 25 to 1000-5000 nM, with from about 50 to about 1000 nM being preferred, and from about 50 to 250 nM being particularly preferred.

Exocytosis assays based on fluorescent dye release rely on release of dye that is taken up passively or actively endocytosed by the cell. Release of dyes taken up by a cell results in decreased cellular fluorescence and presence of the dye in the cellular medium, thus providing two basis for measuring dye release. For example, styryl dyes taken up into cells by endocytosis is released into the cellular media by exocytosis, resulting in decreased cellular fluorescence and presence of the dye in the medium. Another dye release assay uses low pH dyes, such as acridine orange, LYSOTRACKER™ red, LYSOTRACKER™ green, and LYSOTRACKER™ blue (Molecular Probes, supra), which stains exocytic granules when dye is internalized by the cell.

Preferential staining of exocytic granules when the vesicles fuse with the cell membrane provides an additional assay for measuring exocytosis. Annexin V, which binds to the phospholipid phosphatidyl serine in a divalent ion dependent manner, specifically binds to exocytic granules present on the cell surface but fails to bind internally localized exocytic granules. This property of Annexin provides a basis for determining exocytosis by the level of Annexin bound to cells. Cells show an increase in Annexin binding in proportion to the time and intensity of the exocytic response. Annexin is detectable directly by use of fluorescently labeled Annexin derivatives (e.g., FITC, TRITC, AMCA, APC, or Cy-5 fluorescent labels), or indirectly by use of Annexin modified with a primary label (e.g., biotin), which is detected using a labeled secondary agent that binds to the primary label (e.g., fluorescently labeled avidin).

Alternatively, in a preferred embodiment the exocytosis indicators are engineered into the cells. For example, recombinant proteins comprising fusion proteins of a granule specific, or a secreted protein, and a reporter molecule are expressed in a cell by transforming the cells with a fusion nucleic acid encoding a fusion protein. This is generally done as is known in the art, and will depend on the cell type. Generally, for mammalian cells, retroviral vectors, including those of the present invention, are preferred for delivery of the fusion nucleic acid. Preferred reporter molecules include, but are not limited to, Aequoria victoria GFP, Renilla muelleri GFP, Renilla reniformis GFP, Renilla ptilosarcus, GFP, BFP, YFP, and enzymes including luciferases (e.g., Renilla, firefly etc.) and β-galactosidases. Presence of the granule protein-reporter fusion protein on the cell surface or presence of secreted protein-reporter fusion protein in the medium indicates the level of exocytosis in the cells. Thus, in one preferred embodiment cells are transformed with retroviral vectors expressing a fusion protein comprising granule specific (e.g., secretory vesicle) protein, such as synaptobrevin (VAMP) or synaptotagmin, fused to a GFP reporter molecule. The cells are monitored for localization of the fusion protein to the cell membrane. By addition of a separation sequence and an second gene of interest comprising a distinguishable reporter or selection gene, cells expressing the fusion protein are readily selected. Moreover, the second gene of interest provides an internal standard to measure level of fusion protein content in the cell. Candidate agents, for example candidate nucleic acids and candidate peptides, introduced into these transformed cells are tested for their ability to affect distribution of the fusion protein. When the granule specific proteins comprises mediators released during exocytosis, such as serotonin, histamine, heparin, hormones, etc., these granule proteins may be identified using specific antibodies.

In another aspect, the present invention also finds use in drug resistance applications. Multiple drug resistance, and hence tumor cell selection, outgrowth, and relapse, leads to morbidity and mortality in cancer patients. The present invention is applicable to a variety of screens for agents counteracting the drug resistance phenotype of cells. In one preferred embodiment, multidrug resistant cells are treated with a library of retroviral expression vectors of the present invention where the first gene of interest comprises candidate agents (e.g., nucleic acids or peptides). When the candidate agents are candidate peptides, fusions with membrane localization sequences can display the peptides either intracellularly or extracellularly. Targeting the candidate peptides to the membrane in a specific orientation may increase the effective molar concentration of the candidate agent to provide sufficient concentrations to affect the activity of membrane localized drug resistance proteins or their regulators. The second gene of interest is a reporter or selection gene, which allows selection of cells expressing the candidate agents. This construct allows identification of bioactive candidate agents that confer drug sensitivity when the cells are exposed to the drugs of interest. The readout can be the onset of apoptosis in these cells, membrane permeability changes, the release of intracellular ions and fluorescent markers. Cells in which multidrug resistance involves membrane transporters can be preloaded with fluorescent transporter substrates, and selection carried out for peptides which block the normal efflux of fluorescent drug from these cells. Candidate libraries are particularly suited to screening for peptides which reverse poorly characterized or recently discovered intracellular mechanisms of resistance or mechanisms for which few or no chemosensitizers currently exist. Similar types of screens may be used to identify cells with increased tolerance to drug toxicity.

In another preferred embodiment, the retroviral vectors are used to confer multidrug resistance on cells by expressing multidrug resistance genes, such as MDR, MRP and BRAP. In one aspect, the fusion nucleic acid may comprise a first gene of interest encoding a multidrug resistance gene, separation sequence, and a second gene of interest encoding a reporter. Expression of the multidrug resistance gene bestows on the cell resistance to a variety of drugs. Candidates agents are then introduced into the cells in the presence of the drug to identify agents sensitizing the cells to the drug. The expression of the reporter allows distinguishing between agents acting on synthesis of the transporter versus agents acting on transporter activity. In many cases, multidrug resistance in cells arises from expression of combinations of multidrug resistance genes (e.g., MDR and MRP). For these situations, the fusion nucleic acid may comprise a first gene of interest encoding a first multidrug resistance gene and the second gene of interest encoding a second multidrug resistance gene. The presence of a separation sequence allows each multidrug resistance protein to function independently and to be expressed in near stoichiometric levels. Candidate agents or mixtures of candidate agents (cocktails) are screened in the presence of a toxic drug to identify agents capable of acting on cellular regulators or drug transporters that are responsible for the multidrug resistance phenotype. These may lead to therapeutic agents that increases the efficacy of traditional chemotherapy, especially in more advanced tumors where multidrug resistance renders chemotherapy ineffective.

In another aspect, the candidate agents are screened for anti-death gene activity. The retroviral vector comprises a death gene, such as Fas receptor, which induces cell death in presence of its cognate ligand. Death genes that do not depend on a ligand, such as caspases and bax, may also comprise the first gene of interest. In cases where the death gene activity does not depend on a ligand, a regulated inducible promoter is preferred to limit expression of the death gene. The death ligand is added or the promoter is induced to promote cell death. Candidate agents are added before or after initiating the death gene activity. Presence of viable cells indicate presence of candidate agents antagonizing death gene activity.

Since different pathways may be involved in promoting cells death, multiple death promoting genes may be expressed using the present invention. Thus, in one embodiment, plurality of caspases known to act in various cell death pathways are expressed. When cell death is dependent on interaction of multiple protein components, for example formation of apoptosome complex comprising caspase 9 and Apaf-1, these combinations of proteins are expressed by the fusion nucleic acids of the present invention. Candidate agents or combinations of candidate agents are then introduced into these cells to screen for agents and cellular targets acting on these death pathways initiated by the combinations of death promoting proteins.

The present invention is also useful for screening agents active against death genes comprising toxins, especially those made by pathogenic organisms. In one preferred embodiment, the first gene of interest comprises a toxin, such as the cholera toxin, linked to a second gene of interest comprising a reporter gene. The promoter is preferably an inducible promoter to limit toxicity arising from basal level expression in the cell. Upon inducing the promoter, synthesis of the toxin gene occurs, resulting in cell death or lowered cell survivability. Candidate agents are added before or after induction, and those agents conferring anti-toxin activity identified. Reporter gene expression provides a measure of toxin gene synthesis.

In yet another embodiment, the retroviral vectors find use in screens for effectors and cellular mediators of cell cycle regulation. It is known that the cell cycle is regulated by complex regulatory pathways involving molecules such as cellular receptors, cyclins, cyclin dependent kinases, cyclin dependent kinase inhibitors, cell division cycle phosphatases, ubiquitin ligases and ubiquitin protease complex, tumor suppressor proteins, and transcription factors. Cell cycle dysregulation is implicated in progression of many tumors and in inappropriate activation of the immune response. To identify candidate peptide agents modulating cell cycle regulation, retroviral vectors comprising candidate agents as a first gene of interest are introduced into cells having senescent or proliferative properties. The second gene of interest is a reporter protein to monitor expression of the peptides, but may also comprise a reporter that communicates the cell cycle status of the cell, for example a GFP fused to a chromatin associated protein (e.g., histones; see Belmont, A. S. (2001) Trends Cell Biol. 11: 250-7; Kimura, H. et al. (2001) J Cell Biol. 153: 1341-53). This allows selecting for candidate agents having specific effects on the cell cycle (see application US 2001/0003042, hereby incorporate by reference).

In another embodiment, the retroviral vectors are employed to express cell cycle regulators or express mutants of cell cycle regulatory proteins, which produces an aberrant cell cycle phenotype in the cells. Examples of cell cycle regulators include, but are not limited to, cellular receptors, cyclins, cyclin dependent kinases, cyclin dependent kinase inhibitors, cell division cycle phosphatases, ubiquitin ligases, ubiquitin proteasome complex, tumor suppressor proteins, and transcription factors regulating expression of cell cycle proteins. These genes of interest may be full length proteins or domains of proteins having cell cycle regulatory activity. In one aspect, the retroviral vectors may comprise a first gene of interest comprising a cyclin (Cln) and a second gene of interest comprising a cyclin dependent kinase (Cdk), which is activated by the cyclin. Expression of the two products in a cell activates Cdk pathways leading to aberrant cell cycle. These cell lines then serve as screening systems for agents which block particular Cdk mediated pathways and also for agents which block Cdk activity. The bioactive candidate agents may function by acting directly on the kinase, for example by affecting association of cyclins and cdk, or indirectly by affecting stability of the cyclins or cdks (e.g., degradation).

In another preferred embodiment, the present methods are used to examine channel function. Voltage gated (e.g., Na⁺, K⁺, Ca⁺² channel etc.) and non-voltage gated (e.g., Cl⁻, Ca⁺² channels, aquaporin etc.) channels function in a wide variety of cellular processes, including ion balance, nerve conduction, exocytosis, neurotransmitter release, osmotic balance, and nervous system development. Consequently, defects in channel function results in a variety of disease states, such as cardiac arrhythmias (defects in K⁺ channels), epilepsy (defects in Na⁺ channels), autosomal dominant polycystic kidney disease (defects in Ca⁺² channels), and abnormal neural organization (defects in K+ channels; see for example Kofuji, P. (1996) Neuron 16: 941-52). In addition, receptors regulating internal ion stores, especially internal Ca⁺² reservoirs regulated by ryanodine receptors, are responsible for diseases such as autosomal dominant cardiomyopathy.

In one preferred embodiment, the retroviral vectors of the present invention are useful in identifying candidate agents which affect channel activity or other regulators of cellular ion fluxes. The fusion nucleic acids may comprise a first gene of interest comprising candidate agents and a second gene of interest encoding a reporter or selection molecule. These nucleic acids are introduced into cell types expressing a specific channel or channel variant and screened for bioactive candidate agents that block, activate, or modulate channel activity. As is well known in the art, assaying channel function can employ a variety of techniques such as voltage clamp, patch clamp, or intracellular ion sensors (e.g., fura-2). Presence of a separation sequence allows monitoring the synthesis of candidate peptides without affecting its biological activity. Bioactive agents are selected that directly or indirectly affect the activity of various channels, including voltage gated channels, Ca⁺² ion channels. sodium-calcium exchange proteins, sodium proton pump function, and sarcolemmal calcium cycling.

Alternatively, in another preferred embodiment, the gene of interest is an intracellular biosensor of ion channel activity. In one aspect, the biosensor may comprise an ion channel fused to a GFP molecule such that its fluorescence properties changes as a function of channel activity. For example, changes in the local environment caused by movement of the voltage sensor domain in the cellular membrane or by the conformational changes in channel protein structure can alter fluorescent properties of GFP fused to the ion channel. Candidate agents are introduced into cells expressing these channel biosensors and examined for modulation of ion channel function. In cases where ion channels are heteromultimeric, the present invention simplifies expression of the heteromultimers in a single cell since the fusion nucleic acids of the present invention permit expression of each channel subunit from a single nucleic acid construct.

In another preferred embodiment, the fusion nucleic acids of the present invention are useful for examining signal transduction pathways involved in disease states, such as tumorigenesis. Mutations or inappropriate expression of genes such as AbI, Src, Ras, Raf, Rb, p53, and others, induce abnormal cell growth phenotype arising from disrupted signal transduction regulation. These transformed cells types provide a platform for identifying candidate agents that affect the disrupted signal transduction pathway. In one aspect, fusion nucleic acids expressing candidate agents are introduced into transformed cells to identify agents that inhibit, enhance, or modulate the transformed phenotype, and hence regulate signal transduction pathway affected in the transformed cell. The cellular targets of the candidate agents are identified to provide a basis for design of therapeutic agents.

In another preferred embodiment, fusion nucleic acids of the present invention are used to produce an aberrant signal transduction phenotype in a cell, which then serves as a platform for identifying candidate agents and cellular targets regulating the induced phenotype. Thus, in one aspect, the fusion nucleic acid may comprise a first gene of interest that produces a dominant phenotype in a cell, such as AbI, Src, Ras, Raf, Rb, p53, or ErbB-2 (HER2/Neu) or variants thereof. Incorporation of a separation sequence and a second gene of interest comprising a reporter or selection gene readily identifies cells expressing the first gene of interest. These nucleic acids are introduced into selected non-transformed cells to generate transformed cell lines (i.e., produce a dominant effect). The separation site operably linked with the reporter or selection gene allows monitoring expression of the oncogene without detrimentally affecting oncogene function. The reporter may be a GFP protein while the selection gene may encode puromycin resistance. Since it is well known that expression of these oncogenes (e.g., AbI, Src, or Ras) in certain cell lines, such as NIH 3T3 cells, causes the cells to hypertransform and detach from the plate, these artificially transformed cells provide a basis for identifying candidate agents affecting a specific signal transduction pathway. For transformed NIH3T3 cells, the detached phenotype affords a convenient screening method since washing separates unattached cells from attached cells. Cells which express a candidate bioactive agent that reverses the transformed phenotype will cause the cells to remain attached to the plate.

Alternatively, combinations of genes of interest acting synergistically to produce a dominant phenotype may be expressed by the fusion nucleic acids of the present invention. In regards to tumorigenesis, it is well known that tumorigenesis is believed to require activation of multiple oncogenes. For example, Ras and Raf oncogenes act (see Cuadrado, A. et al. (1993) Oncogene 8: 244348) synergistically to transform cells via the Ras signaling pathway. Another illustration of cooperative effects between cellular proteins in producing a dominant phenotype is the interaction of mutant β-catenin and Tcf/Lef protein. Stable interaction of the two proteins leads to constitutive activation of Tcf mediated transcription, which ultimately leads to progression of colon cancer (see for example, Kolligs, F. T. (1999) Mol. Cell. Biol. 19: 5696-706). The fusion nucleic acids of the present invention allows expressing combinations of these oncogenes within a single cell, thereby providing a means to the generate transformed cells not achievable with expression of a single oncogene. Once these transformed cells are available, screens may be conducted for candidate agents that specifically reverse, enhance, or modulate the dominant phenotype caused by the co-expressed proteins.

In another preferred embodiment, the present invention finds use in immunology, inflammation, and allergic response applications. For example, activation of B-cells initiates various facets of humoral immunity, including immunoglobulin synthesis and antigen presentation by B-cells. Activation is mediated by engagement of the B-cell receptor (BCR), for example by binding of anti-IgM F(ab′) fragments, which activates several signal transduction pathways leading to specific responses by the B-cell, including apoptosis, expression of cell surface marker CD69, and modulation of IgH promoter activity. Thus, in one aspect, the retroviral vectors of the present invention are useful for introducing candidate agents, such as libraries of cDNAs, candidate nucleic acids, and candidate peptides into appropriate B-cell lines, such as Ramos Human B-cell lines or M12.4, to identify various effectors of the signaling pathways mobilized by B-cell receptor engagement. The effector may be the candidate agents themselves or the cellular targets of the candidate agents, and the assay may comprise determining the level of CD69 cell surface marker (e.g., by fluorescently labeled anti-CD69 antibody and FACS selection of cells expressing high levels of CD69) or inhibition of apoptotic pathway following receptor activation.

In another aspect, the present invention is useful as indicators of B-cell receptor mediated signal transduction. In one preferred embodiment, the retroviral vector may comprise an IgH promoter operably linked to a first gene of interest comprising a reporter gene, a separation sequence, and a second gene of interest comprising a second reporter or selection gene. For example, the genes of interest may comprise combinations such as GFP and HBEGF, which provides selection based on GFP expression and diptheria toxin mediated killing. This and other configurations provides sensitive monitoring of BCR activation by the detecting IgH promoter activity. Candidate agents are introduced into these cells to identify agents that activate or suppress BCR mediated signal transduction, as reflected by changes in IgH promoter activity. Expression of the candidate agents may be under the control of an inducible promoter, such as tetP., thus limiting any detrimental effect on the cell by constitutively expressing candidate agents. Inducible expression of candidate agents also provides a basis for distinguishing between altered cellular phenotypes caused by somatic mutations and candidate agents. Generally, cells used in this type of screen will also a comprise fusion nucleic acid expressing the tetracyclin regulatable transactivators (Goose, N. M. et al. (1995) Science 268: 1766-69).

In another aspect, the present invention is applicable to cell mediated immunity. Effective cellular immune response against intracellular pathogens or tumors relies on generation of CD8⁺ cytotoxic lymphocytes (CTL). CTLs become activated by recognizing complexes of antigen-MHC-I molecules displayed on the surface of antigen presenting cells (APC), such as monocytes, macrophages, B-cells, and dendritic cells. Recognition of a separate group of peptides complexed with MHC II molecules on the APCs results in secretion of cytokines required for expansion and maturation of T-cells. T-cell activation also requires an additional signal initiated by an APC associated costimulatory molecule (B7 ligands), which binds the CD28 receptor on T-cells. The absence of a costimulatory signal or the activation of CTLA-4 receptors on T-cells by binding of the B7 ligand induces a state of “anergy” in which T-cells are rendered non-responsive to antigen stimulation. Thus, the pathways for T-cell activation/inactivation provide various approaches to identify candidate agents and cellular mediators of T-cell mediated immune response.

In one embodiment, the retroviral vectors of the present invention may comprise a first gene of interest comprising a cDNA library made from tumor cells. The cDNA is preferably a subtracted library of normal cells and tumor cells while the second gene of interest comprises a reporter gene, such as a FACS selectable GFP protein. In this way, the subtracted cDNA library comprises tumor antigens preferentially expressed on tumor cells (see Byrne, J. A. (1995) Cancer Res. 55: 2896-903). The retroviral vectors are then introduced into APCs, preferably dendritic cells, which are the main initiators of the immune response, and combined with naive T-cells to form activated T-cells (Timmerman, J. M. (1999) Annu. Rev. Med. 50: 507-529). Killing of tumor cells by the T-cells are examined to determine the repertoire of tumor antigens capable of eliciting efficient CTL mediated killing. Once an initial set of CTL activating antigens are identified, biased random peptides are made to find specific peptides functioning as tumor vaccines capable of eliciting strong CTL responses. In an alternative embodiment, the second gene of interest on the fusion nucleic acid may comprise a costimulatory molecule (for example B7 ligands) to strongly activate T-cells during antigen presentation by the APCs.

In another application to immunotherapy, the first gene of interest may comprise random peptide sequences while the second gene of interest comprises either a tumor antigen present on tumor cells (e.g., melanocarcinoma antigen MAGE-1) or a cytokine (e.g., interleukin-2) needed to promote maturation of T-cells. Peptide candidates that stimulate or inhibit T-cell maturation in presence of cytokines or tumor antigen are then selected. These bioactive candidate peptides may act through CD80 or CTLA-4 receptors or act on the signal transduction pathways mediated by these receptors. These agents are then used for identify cellular targets responsible for enhancing T-cell activation, which may lead to therapeutic compounds useful for treating tumors, or inhibiting T-cell proliferation (e.g., , induced anergy) for counteracting rejection of organ/cell transplants, for alleviating autoimmune diseases, or ameliorating inflammatory reactions.

Finally, the retroviral vectors are useful in a variety of gene therapy applications. The retroviral vectors of the present invention allows introduction of genes of interest to complement mutations or deletions of natural analogs in the host organism or cell. Accordingly, in one aspect, cells of a host suffering from a genetic defect are isolated and exposed to retroviral vectors containing a first gene of interest comprising a normal gene that complements the mutated or deleted gene of the host. A second gene of interest comprises a reporter protein, thus providing a basis for isolating only those cells expressing the normal gene. Reporter genes are chosen to have minimal effect on eliciting an immune response against cells expressing the reporter protein. The cells are then reintroduced into the host organism. Preferred cells are stem cells obtained from the host. A variety of genetic disorders will be amenable to such treatment, including various forms of muscular dystrophy, cystic fibrosis, lysozomal storage disease (e.g., Gaucher's disease), adenosine deaminase, etc.

In another embodiment, the retroviral vectors are used to express multiple protein products useful for gene therapy, especially for cancer therapy. These may involve introduction of genes mutated in various cancers or introduction of combinations of genes affecting the proliferative potential of the tumors, such as tumor suppressor genes p53 and retinoblastoma protein (Rb). It is known that expression of normal copies of the tumor suppressor genes can reduce the proliferative potential in cancerous cells containing mutated p53 and Rb.

In yet another embodiment, the retroviral of the present invention find use in gene therapy directed to enhancing immune response against tumor cells. In one aspect, a retroviral vector may comprise a costimulatory molecule (B7 ligand) required for CTL activation as the first gene of interest and a reporter molecule for FACs selection of expressing cells as the second gene of interest. A separation sequences is used to generate separate first and second genes of interest. Tumor cells obtained from patients are exposed to these retroviral vectors, and cells stably expressing the costimulatory ligand are isolated, for example by FACs. Reintroduction of the cells into the patient can enhance CTL action against the tumor cells.

Alternatively, the retroviral vectors may comprise a first gene of interest comprising a costimulatory molecule while the second gene of interest comprises a cytokine needed for T-cell proliferation, such as interleukin 2 (IL-2), or interleukin 12 (IL-2). Since IL-12 is heteromultimeric, additional separation sequences and gene of interest may be used to express the heteromultimeric cytokine. Introduction of these constructs into tumor cells, or APCs isolated from tumor cells, can enhance CTL action against the tumor cells. As can be appreciated by those skilled in the art, numerous combinations of genes of interest may be used to enhance the immune response.

It is understood by the skilled artisan that the steps for constructing the fusion nucleic acids, retroviral libraries, and cellular libraries can be varied according to the options provided herein. Those skilled in the art may modify according to the skill in the art

The following examples serve to more fully describe the manner of using the above-described invention, as well as to set forth the best modes contemplated for carrying out various aspects of the invention. It is understood that these embodiments in no way serve to limit the scope of this invention. All references cited herein are incorporated by reference.

### EXAMPLES

### Example 1

### General Procedures

Cells Cultures: Jurkat lymphoblastic T cells expressing the MMLV-ecotropic receptor (JE) are described in Hitoshi, et al. (1998) Immunity 8: 461-71) and cultured in RPMI medium (Invitrogen, San Diego, Calif.) supplemented with 10% heat-inactivated fetal bovine serum (JRH, Lenexa, Kans.), 100 IU/ml penicillin, and 100 ug.ml streptomycin. A549.tTA cells, a lung carcinoma cell line that constitutively expresses the tet transactivator protein (tTA) was maintained in F12K medium supplemented with 10% heat-inactivated fetal bovine serum, 100 IU/ml penicillin, and 100 ug/ml streptomycin. 293-human embryonic kidney cells were grown in DMEM/10% fetal bovine serum, 100 IU/ml penicillin, 100 ug/ml streptomycin.

Retroviral transduction: All retroviral constructs were derived from CRU5-GFP retroviral vector, which is described below. Production of infectious retroviral vector particles in the 293-based Phoenix A packaging cells and infection was carried out as described in Swift, et al., In Current Protocols in Immunology (J. E. Coligan, A. M. Kruisbeek, D. H. Marguiles, E. M. Shevach, and W. Strober, Eds.), Vol. 10 17C, pp1-17, Wiley, N.Y. Phoenix A packaging cells were transfected with retroviral plasmid constructs and incubated for 24 hrs. Jurkat or A549 culture medium was added and virus collected after 24 hrs. Infections were carried out with 0.45 um-filtered virus-containing medium by spin infection.

Flow cytometry: Flow cytometric analysis was conducted on a FACSCaliber flow cytometer (BD-Biosciences, Franklin Lakes, N.J.). FACS data was analyzed using WinList (Verity Software House, Topsham, Me.) analysis program.

### Example 2

### Construction and Expression of Retroviral Vectors Comprising Fusion Nucleic Acids Expressing Separate Reporter Protein and Selection Protein

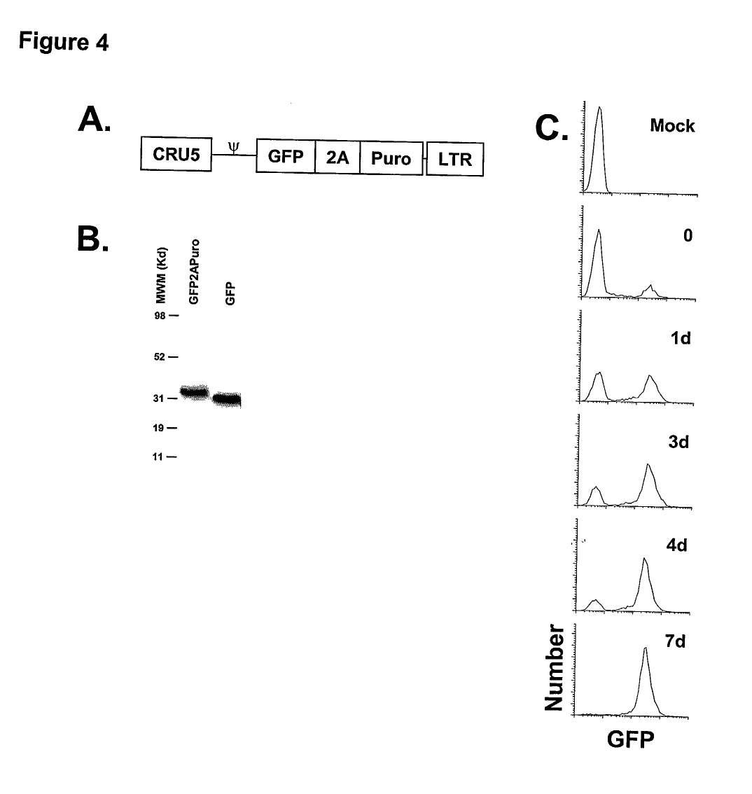

Construct CRU5-GFP-2A-Puro was made in CRU5-GFP retroviral vector (Naviaux, R. K. et al. (1996) J. Virol. 70: 5701-05), which carries a composite CMV promoter fused to the transcriptional start site of the MMLV R-U5 region of the LTR, an extended packaging sequence (T), deletion of the MMLV Gag start ATG, and multiple cloning regions encoding EGFP (BD-ClonTech, Palo Alto, Calif.). The CRU5-GFP-2A-Puro was created by inserting a 2A-encoding linker (5′-GAATTCGGAGGTGGCAGCGGTGGCGGTCAGCTGTTGAATTTTGACCTTCTTAAACTTGCGGGAG ACGTCGAGTCCAACCCTGGGCCCACCACCACCATGG) downstream of the GFP sequence that encodes the in-frame sequence (EF)GGGSGGGQLLNFDLLKLAGDVESNPGP(TTTM) containing the FMDV 2A sequence flanked by 5′ (EcoRI) and 3′ (BstXI) cloning sites. The puromycin phosphotransferase sequence was cloned in-frame with the GFP-2A open reading frame via the BstXI site in the linker and a downstream NotI site in the vector (FIG. 4A).

Jurkat T cells were infected with CRU5-GFP-2A-Puro and assayed for 2A mediated processing efficiency by Western blotting with anti-GFP antisera. Transduced Jurkat cell lysates were prepared in lysis buffer (50 mM HEPES, pH 7.4, 150 mM NaCl, 5 mM EDTA, 5 mM EGTA, 1% Triton X-100) containing complete protease inhibitor cocktail (Boehringer-Mannheim, Chicago, Ill.). Cleared lysates were resolved on SDS-polyacrylamide gels and blotted according to manufacturers recommendations (Novex, San Diego, Calif.). The blots were incubated with anti-GFP polyclonal antibody (Molecular Probes, Eugene, Oreg.) and bound antibody detected using enhanced chemiluminescence (ECL plus; Amersham, Chicago, Ill.). The GFP 2A-Puro expressing cells produce a GFP species which migrates slower than the native GFP due to the additional 18 amino acids of the FMDV-2A (FIG. 4B). Higher molecular weight species were not observed suggesting efficient processing of the GFP-2A-Puro peptide.

Jurkat cells infected with CRU5-GFP-2A-Puro at low MOI, which produces detectable GFP fluorescent cell populations, were selected with puromycin at 2 ug/ml. Aliquots of the CRU5-GFP-2A-Puro infected cells were analyzed by FACS for concomitant enrichment of GFP fluorescence (FIG. 4C). After 7 days of puromycin selection, the GFP-2A-Puro expressing cell population was >99.9% GFP positive, congruent with complete co-selectability of the GFP and puromycin phosphotransferase activities.

### Example 3

### Construction and Expression of Retroviral Vectors Comprising Fusion Nucleic Acids Expressing Proteins Targeted to Distinct Subcellular Compartments

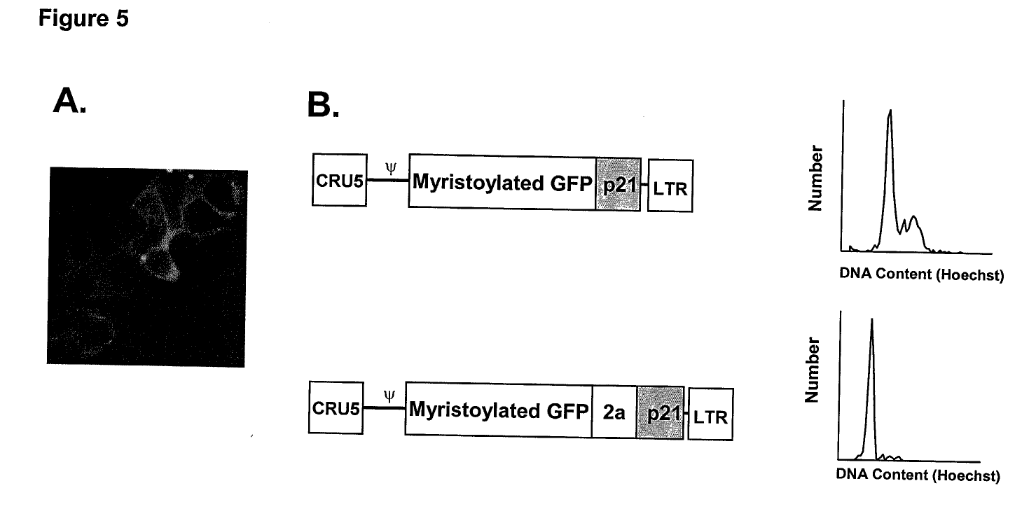

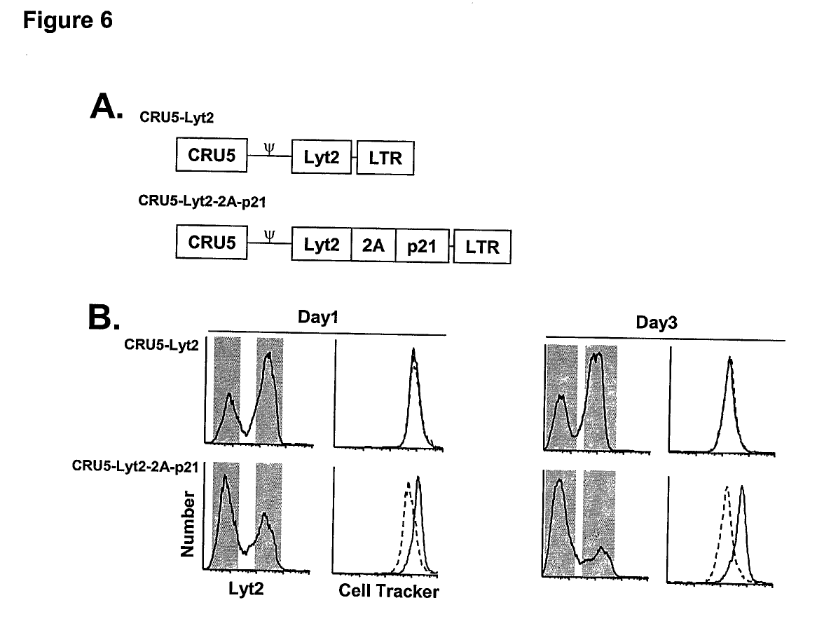

The retroviral construct CRU5-myrGFP-p21 was made in a CRU5-GFP retroviral vector (FIG. 5B). The construct comprises a fusion nucleic acid in which GFP fusion protein with an N-terminal myristolation sequence MGQSLTTH (5′-ATGGGACAATCGCTAACAACCCAT) found in Rasheed rat sarcoma virus Gag protein is fused in frame to the GFP-p21 sequence of a CRU5-Gp21 vector (Lorens, J. B. et al. (2000) Molecular Therapy 1: 438-447) by PCR. Presence of a bipartite nuclear localization signal at the C terminus of p21 targets the protein to the nucleus. Construct CRU5-myrGFP-2A-p21 is identical to CRU5-myrGFP-p21 except that a Type 2A separation sequence (see Experiment 1) is inserted between the GFP and p21 sequences via EcoRI and BstXI sites. Transfection of HEK293 cells results in membrane localized fluorescence for both myrGFP-p21 and CRU5-myrGFP-2A-p21 (FIG. 5A). Jurkat cells infected by the either construct were assayed for effects on the cell cycle by FACS. Infected cells were stained with Hoechst 33342 and the GFP-expressing fraction examined for DNA content (FIG. 5C). The CRU5-myrGFP-p21 expressing cells shows a cell cycle distribution indistinguishable from control infected or non-GFP-expressing cells. In contrast, the CRU5-myrGFP-2A-p21 expressing cells cell cycle arrest at G1, consistent with processing and nuclear localization of p21 protein.

### Example 4

### Expression of Retroviral Vectors Comprising Fusion Nucleic Acids Expressing Separate Reporter Protein and Dominant Effector Protein

The CRU5-Lyt2-2A-p21 is a retroviral construct made in a CRU5-GFP vector and contains Lyt2a, which is a truncated mouse CD8 cell surface marker. The construct also comprises p21 protein, an inhibitor of cyclin dependent kinases. The Lyt2α contains a signal peptide and is targeted to the plasma membrane while p21 is a nuclearly localized protein. CRU5-Lyt2-2A-p21 was made by inserting the 2A-p21 sequence from CRU5-GFP-2A-p21 into CRU5-Lyt2, which has the GFP sequence of CRU5-GFP replaced with the mouse Lyt2α. A human lung carcinoma cell line, A549, was infected with retroviruses comprising CRU5-Lyt2 control construct (containing only the Lyt2 peptide) or viruses comprising CRU5-Lyt2-2A-p21 and assayed by FACS for Lyt2 cell surface localization and cell cycle progression.

For cell cycle FACS assay, the infected cells were pulse labeled with cell tracker dye PKH26. Transduced Jurkat cells were pelleted and resuspended at 10⁶ cells/ml in RPMI. One volume (1 ml) of 4 uM PKH26 cells tracking dye (Sigma, St. Louis, Mo.) was added to the cells and incubated at 25° C. for 5 min. The cell suspension was diluted 5 fold in complete RPMI, washed twice with complete RPMI, and incubated at 3×10⁵ cells/ml in 6 well plates. The cells were subsequently stained with anti-Lyt2 antibodies (Pharmigen) and subjected to flow-cytometric analysis on a MoFlo cytometer (Cytomation, Fort Collins, Colo.).

Cell Tracker PKH is a membrane labeling dye used to identify cell cycle status of the cell population. Arrested cells remain cell tracker dye bright, while cycling cells dilute the signal at each cell division, thus exhibiting lower fluorescence. Lyt2 expressing and non-expressing cells were gated and correlated with cell tracker fluorescence. At both 24 and 72 hr time points, the Lyt2 expressing and non-expressing subpopulations were indistinguishable with respect to cell tracker fluorescence. In contrast, the Lyt2-expressing subpopulation of the CRU5-Lyt2-2A-p21 expressing cells showed higher celltracker mean fluorescence relative to Lyt2 negative cells, thus indicating nuclear localization of p21 and a resultant phenotype of growth arrest.

We claim:

1. A retroviral vector comprising fusion nucleic acids comprising:
a) a promoter;
b) a different first gene of interest;
c) a protease recognition sequence; and
d) a second gene of interest.

2. A retroviral vector comprising fusion nucleic acids comprising:
a) a promoter;
b) a different first gene of interest;
c) a Type 2A sequence; and
d) a second gene of interest.

3. A retroviral vector according to claim 1 or 2, wherein said first or second gene of interest comprises a reporter gene.

4. A retroviral vector according to claim 3, wherein said reporter gene is a GFP.

5. A retroviral vector according to claim 1 or 2, wherein said first or second gene of interest comprises a selection gene.

6. A retroviral vector according to claim 1 or 2, wherein said first or second gene of interest comprises nucleic acid encoding a dominant effector protein.

7. A retroviral vector according to claim 1 or 2, wherein said first or second gene of interest comprises a nucleic acid encoding a random peptide.

8. A retroviral vector according to claim 1 or 2, wherein said first and second gene of interest comprise nucleic acids encoding random peptides.

9. A retroviral vector according to claim 7 or 8, wherein said random peptide is biased.

10. A retroviral vector according to claim 1 or 2, wherein said first or second gene of interest comprises cDNA.

11. A retroviral vector according to claim 10, wherein said first or second gene of interest comprises a cDNA fragment.

12. A retroviral vector according to claim 1 or 2 wherein said first or second gene of interest comprises a fragment of genomic DNA.

13. A retroviral vector according to claim 1 or 2 wherein at least one of said gene of interest comprises a multiple cloning site (MCS).

14. A retroviral vector according to claim 1 or 2 wherein said both genes of interest comprise reporter genes.

15. A retroviral vector according to claim 1 or 2 wherein said both genes of interest comprise selection genes.

16. A composition comprising a library of retroviral vectors each comprising:
a) a promoter;
b) a different first gene of interest;
c) a separation site; and
d) a second gene of interest.

17. A composition according to claim 16 wherein said separation site comprises a Type 2A sequence.

18. A composition according to claim 16 wherein said separation site comprises a nucleic acid encoding a protease cleavage site.

19. A composition according to claim 16 wherein said separation site comprises an internal ribosome entry sequence (IRES).

20. A composition according to claim 16 wherein each of said second genes of interest comprises a reporter gene.

21. A composition according to claim 16 wherein said reporter gene comprises a GFP gene.

22. A composition according to claim 16 wherein each of said second genes of interest comprises a selection gene.

23. A composition according to claim 16 wherein said each of said second genes of interest comprises a nucleic acid encoding a dominant effector protein.

24. A composition according to claim 16 wherein said each of said first genes of interest comprises a nucleic acid encoding a random peptide.

25. A composition according to claim 24 wherein said random peptide is biased.

26. A composition according to claim 16 wherein said each of said first genes of interest comprises a cDNA.

27. A composition according to claim 26 wherein said cDNAs comprise cDNA fragments.

28. A composition according to claim 16 wherein said each of said first genes of interest comprises a genomic DNA fragment.

29. A composition according to claim 16 wherein both of said genes of interest comprises a nucleic acid encoding a random peptide.

30. A composition according to claim 16 wherein at least one of said genes of interest comprises a multiple cloning site.

31. A cellular library comprising a library of retroviral vectors each comprising a fusion nucleic acid comprising:
a) a promoter;
b) a different first gene of interest;
c) a separation site; and
d) a second gene of interest.

32. A method of screening cells for altered phenotypes comprising
a) providing a cellular library comprising a library of retroviral vectors each comprising a fusion nucleic acid comprising
i) a promoter;
ii) a different first gene of interest;
iii) a separation site; and
iv) a second gene of interest;
b) adding at least one candidate agent to said cellular library; and
c) screening said cellular library for a cell exhibiting an altered phenotype.

33. A method according to claim 32 further comprising d) isolating said cell.

34. A method according to claim 33 further comprising e) identifying the candidate agent responsible for said altered phenotype.

35. A method according to claim 32, wherein a library of candidate agents is added to said cellular library.

36. A method according to claim 35, wherein said library of candidate agents comprise a library of small molecules.

37. A method according to claim 35, wherein said library of candidate agents comprise nucleic acids encoding random peptides.

38. A method according to claim 37, wherein said random peptides are biased.

39. A method according to claim 35, wherein said library of candidate agents comprise cDNAs.

40. A method according to claim 39, wherein said cDNAs comprise cDNA fragments.

41. A method according to claim 32, wherein said library of candidate agents comprise fragments of genomic DNA.
